(19) United States
(12) Patent Application Publication
(10) Pub. No.: US 2026/0021144 A1
(43) Pub. Date: Jan. 22, 2026

(54) SYSTEMS FOR CELL PROGRAMMING TOWARDS CHONDROGENIC LINEAGE AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US)

(21) Appl. No.: 19/285,507

(22) Filed: Jul. 30, 2025

Related U.S. Application Data
(63) Continuation of application No. PCT/US24/14094, filed on Feb. 1, 2024.
(60) Provisional application No. 63/482,721, filed on Feb. 1, 2023.

(51) Int. Cl. A61K 35/35; C12N 5/077; C12N 15/85

(52) U.S. Cl. CPC A61K 35/35; C12N 5/0655; C12N 15/85; C12N 2800/107

(57) ABSTRACT

Provided herein are systems of modulating gene expression, methods of use thereof, and cells engineered thereof for the purpose of differentiating cells, for example chondroprogenitor cells.

### CROSS-REFERENCE

This application is a continuation of International PCT Application No. PCT/US24/14094, filed Feb. 1, 2024, which claims the benefit of U.S. Provisional Patent Application No. 63/482,721, filed on Feb. 1, 2023, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Jul. 30, 2025, is named 61684-710_310_SL.xml and is 1,984,169 bytes in size.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell. In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species and cell types.

### SUMMARY

The present disclosure provides methods and systems for programming a cell, e.g., to elicit a desired response in the cell. Systems and methods of the present disclosure can promote conversion of a cell from one type to another. Systems and methods of the present disclosure can utilize a genetic circuit to control a cascade of a plurality of desired expression and/or activity profiles of a plurality of genes in the cell to affect this conversion. Systems and methods of the present disclosure can utilize heterologous proteins and/or nucleic acid molecules as building blocks of such genetic circuit.

In some embodiments, the present disclosure provides a method for conversion of a plurality of stem cells into a plurality of chondrogenic cells, via modulation of expression levels of a plurality of distinct target genes comprising a first homeobox protein and a second homeobox protein, the method comprising: (a) contacting a first polynucleotide sequence in the plurality of stem cells by a first heterologous gene regulating moiety to modulate expression level of the first homeobox protein that is operatively coupled to the first polynucleotide sequence; and (b) contacting a second polynucleotide sequence in the plurality of stem cells by a second heterologous gene regulating moiety, to modulate expression level of the second homeobox protein that is operatively coupled to the second polynucleotide sequence.

In some embodiments, the present disclosure provides a method for conversion of a plurality of stem cells into a plurality of chondrogenic cells, via modulation of expression levels of a plurality of distinct target genes comprising a first distinct target gene and a second distinct target gene, the method comprising: (a) contacting a first polynucleotide sequence in the plurality of stem cells by a first heterologous gene regulating moiety to modulate expression level of the first distinct target gene that is operatively coupled to the first polynucleotide sequence; and (b) contacting a second polynucleotide sequence in the plurality of stem cells by a second heterologous gene regulating moiety, to modulate expression level of the second distinct target gene that is operatively coupled to the second polynucleotide sequence, wherein a combination of the first and second distinct target genes is: (i) a first homeobox protein and a second homeobox protein that are different; (ii) two different members selected from the group consisting of a homeobox protein, a T-box transcription factor (TBX), and a basic helix-loop-helix transcription factor (bHLH); or (iii) a first member selected from the group consisting of the homeobox protein, the TBX, and the bHLH, and a second member comprising SOX or collagen.

In some embodiments, the present disclosure provides a method for conversion of a plurality of stem cells towards chondrogenic differentiation, the method comprising: contacting a polynucleotide sequence in the plurality of stem cells by a heterologous gene regulating moiety to modulate expression level of a target gene that is operatively coupled to the polynucleotide sequence, wherein, within less than 7 days following the contacting, a conversion rate from the plurality of stem cells to chondrogenic cells is characterized to be at least about 30%.

In some embodiments, the present disclosure provides a method for treating a subject in need thereof, the method comprising: administering a plurality of chondrogenic cells to the subject, wherein the plurality of chondrogenic cells is prepared by subjecting a plurality of stem cells to ex vivo differentiation, wherein, within less than 7 days of the ex vivo differentiation, a conversion rate from the plurality of stem cells to the plurality of chondrogenic cells is characterized to be at least about 30%.

In some embodiments, the present disclosure provides a system for conversion of a plurality of stem cells into a plurality of chondrogenic cells, via modulation of expression levels of a plurality of distinct target genes comprising a first homeobox protein and a second homeobox protein, the system comprising: (a) a first heterologous gene regulating moiety configured to bind a first polynucleotide sequence in the plurality of stem cells to modulate expression level of the first homeobox protein that is operatively coupled to the first polynucleotide sequence; and (b) a second heterologous gene regulating moiety configured to bind a second polynucleotide sequence in the plurality of stem cells to modulate expression level of the second homeobox protein that is operatively coupled to the second polynucleotide sequence.

In some embodiments, the present disclosure provides a system for conversion of a plurality of stem cells into a plurality of chondrogenic cells, via modulation of expression levels of a plurality of distinct target genes comprising a first distinct target gene and a second distinct target gene, the system comprising: (a) a first heterologous gene regulating moiety configured to bind a first polynucleotide sequence in the plurality of stem cells to modulate expression level of the first distinct target gene that is operatively coupled to the first polynucleotide sequence; and (b) a second heterologous gene regulating moiety configured to bind a second polynucleotide sequence in the plurality of stem cells to modulate expression level of the second distinct target gene that is operatively coupled to the second polynucleotide sequence, wherein a combination of the first and second distinct target genes is: (i) a first homeobox protein and a second homeobox protein that are different; (ii) two different members selected from the group consisting of a homeobox protein, a T-box transcription factor (TBX), and a basic helix-loop-helix transcription factor (bHLH); or (iii) first member selected from the group consisting of the homeobox protein, the TBX, and the bHLH, and a second member comprising SOX or collagen.

In some embodiments, the present disclosure provides a system for conversion of a plurality of stem cells towards chondrogenic differentiation, the system comprising: a heterologous gene regulating moiety configured to bind a polynucleotide sequence in the plurality of stem cells to modulate expression level of a target gene that is operatively coupled to the polynucleotide sequence, wherein, within less than 7 days following the contacting, a conversion rate from the plurality of stem cells to chondrogenic cells is characterized to be at least about 30%.

In some embodiments, the present disclosure provides a composition for treating a subject in need thereof, the composition comprising: a plurality of chondrogenic cells prepared by subjecting a plurality of stem cells to ex vivo differentiation, wherein, within less than 7 days of the ex vivo differentiation, a conversion rate from the plurality of stem cells to the plurality of chondrogenic cells is characterized to be at least about 30%.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic of the heterologous genetic circuit. An activating moiety initiates the circuit and can activate a gate unit. A gate unit is comprised of a gate moiety and/or a gene regulating moiety.

FIG. 2A depicts the developmental progression from an iPSC to a cartilage cell, including genes known to affect cartilage cell development at different stages. FIG. 2B depicts an example of a heterologous gene circuit that differentiates chondroprogenitor cells from mesoderm cells.

FIG. 3 depicts an exemplary heterologous genetic circuit.

FIG. 4 shows a scatter plot (e.g., a volcano plot) to identify one or more heterologous genetic circuits that induced the stem cell to chondroprogenitor cell conversion.

FIGS. 5A-5D shows examples of chondrogenic progenitor cell marker analysis data utilized to generate the scatter plot in FIG. 4.

FIGS. 6A-6B shows that top performing heterologous genetic circuits convert approximately 60% of cells to chondrogenic progenitor cells in four days. FIG. 6C depicts a summary of the data from FIGS. 6A-6B.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most,” “up to,” “no more than,” “less than,” or “less than or equal to” precedes the first numerical value in a series of two or more numerical values, the term “at most,” “up to,” “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “guide nucleic acid,” “guide nucleic acid molecule,” and “gNA” as used interchangeably herein, generally refer to 1) a guide sequence that can hybridize to a target sequence or 2) a scaffold sequence that can interact with or complex with a nucleic acid guide nuclease. A guide nucleic acid can be a single-guide nucleic acid (e.g., sgRNA) or a double-guide nucleic acid (e.g., dgRNA). sgRNA can be a single RNA molecule that contains both a scaffold tracrRNA and a crRNA which can be complementary to the target sequence. Alternatively, dgRNA can be a single RNA molecule that contains a crRNA annealed to a tracrRNA through a direct repeat sequence.

The use of the alternative (e.g., “or”) should be understood to mean either one, both, or any combination thereof of the alternatives. The term “and/or” should be understood to mean either one, or both of the alternatives.

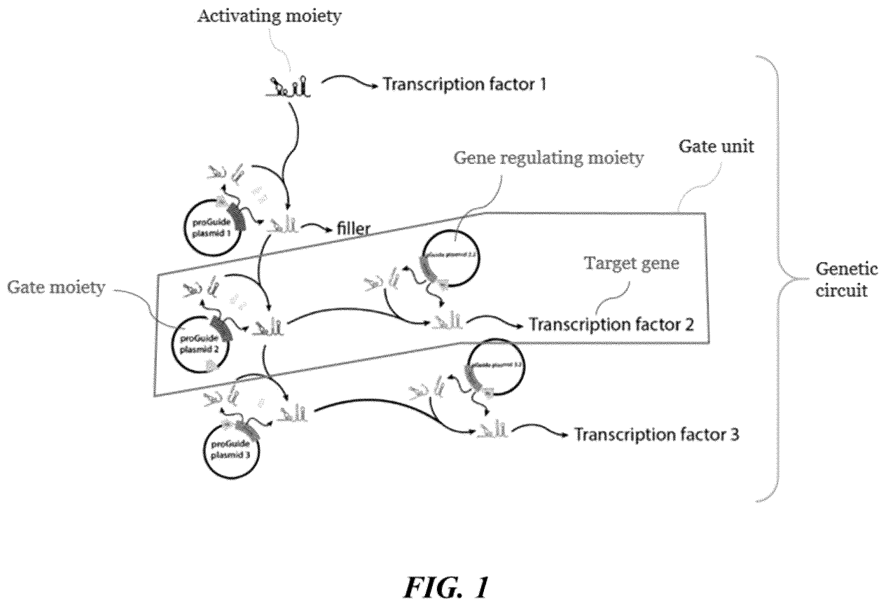

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 1).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units is activatable by an activating moiety (e.g., a heterologous input to the cell) to activate other gate units of the plurality of gate units (e.g., simultaneously at once, sequentially in a cascading manner, etc.) (FIG. 1). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 1). The terms “heterologous genetic circuit,” “HGC,” “genetic circuit,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription/translation regulatory region, or series of regions, which the genetic switch acts on. The input for a gate unit can be an activating moiety and/or another gate unit. The output for a gate unit can be to activate another gate unit, to de-activate another gate unit, to affect a target gene, and/or a combination of any of the above. For example, a gate unit can be comprised of a plurality of gate moieties and/or a plurality of gene regulating moieties (FIG. 1).

The term “activating moiety,” as referred to herein, generally refers to a moiety that can activate plurality of genetic circuits and/or a plurality of gate units. An activating moiety can be a heterologous input to a cell. For example, activating moieties can include, but are not limited to, a guide nucleic acid molecule (e.g., a gRNA) or other nucleic acid, polypeptides, polynucleotides, small molecules, light, or a combination thereof.

For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties. The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively or in addition to, a gate moiety can activate and/or deactivate another gate unit of the genetic circuit (FIG. 1). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. Alternatively or in addition to, a gate moiety can activate and/or deactivate another gate unit of the genetic circuit (FIG. 1).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 1). For example, a gene editing moiety can regulate expression of a gene by editing a nucleic acid sequence (e.g. CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g. CRISPR-Cas-inspired RNA targeting systems). Alternatively, a gene editing moiety can repress translation of a gene (e.g. Cas13).

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g. Cas12).

In some cases, a plasmid can encode a non-functional form of a gene editing moiety. The plasmid can be activated (e.g., genetically modified) to express a functional form of the gene editing moiety, e.g., via activation of a functional gate moiety. For example, the plasmid can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited (e.g., cleaved at one or more sites, then repaired via endogenous mechanisms (e.g., homologous recombination, nonhomologous end joining) to allow expression of a functional form of the gene editing moiety (e.g., a functional form of the guide nucleic acid molecule with specific binding to the target gene of the cell), to permit modulation of the target gene in the cell.

In some cases, a gene regulating moiety can comprise a nucleic acid molecule (e.g., a guide nucleic acid molecule that forms a complex with an endonuclease, such as a Cas protein). Alternatively or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

The abovementioned Cas proteins can form a complex with a guide nucleic acid (gNA (e.g., a guide RNA (gRNA)) and utilize the gNA to specifically bind to a target polynucleotide sequence (e.g., a target DNA sequence, a target RNA sequence). Accordingly, in some cases, such Cas proteins may be referred to as a “NA-guided nuclease” (e.g., RNA-guided nuclease). As used herein, the term “guide nucleic acid” (gNA) can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence”. A nucleic acid-targeting segment may comprise a sub-segment that may be referred to as a “protein binding segment” or “protein binding sequence” or “Cas protein binding segment” or “scaffold sequence.”

A gene regulating moiety can be a transcriptional modulator system (e.g., a gene repressor complex or a gene activator complex). For example, a gene regulating moiety can be a gene repressor complex comprising a dCas protein operatively coupled to (e.g., coupled to or fused with) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASC1, JMJD2D, JARID1A, RBP2, JARID1B/PLU-1, JARIDIC/SMCX, JARIDID/SMCY, HDAC1, HDAC2, HDAC3, HDAC8, HDAC4, HDAC5, HDAC7, HDAC9, SIRT1, SIRT2, HDACl1, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SET1A, SET1B, MLL1, MLL2, MLL3, MLL4, MLL5, ASH1, SYMD2, NSD1, JHDM2a, JHDM2b, UTX, JMJD3, GCN5, PCAF, CBP, p300, TAF1, TIP60/PLIP, MOZ/MYST3, MORF/MYST4, SRC1, ACTR, P160, CLOCK, TET1CD, TET1, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TET1CD), TET1, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

A gene regulating moiety can comprise an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas9. In some cases, an endonuclease can be a deactivated Cas (e.g., dCas, dCas9).

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. During transient expression, episomal DNA can be transferred to daughter cells, but since episomal DNA is not replicated, it is not permanently heritable and will dilute out over time. In contrast, stable expression of a transfected gene can occur when the gene is co-transfected with another gene that confers a selection advantage to the transfected cell. During stable expression, plasmids can have a DNA replication element that allows them to be inherited or integrated into the genome. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [aS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G] dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX] ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “dedifferentiation” or “de-differentiation” generally refers to a process by which a specialized, committed, or partially specialized cell loses the features of the specialized cell (e.g., a chondrogenic cell). A dedifferentiated cell or dedifferentiation-induced cell is one that has taken on a less specialized position within the lineage of a cell (e.g., a stem cell or a progenitor cell). A dedifferentiated cell (e.g., a stem cell or a progenitor cell) can subsequently differentiate into a different cell type or can revert to a less differentiated cell type.

The term “pluripotent” generally refers to the ability of a cell to form all lineages of the body or soma (e.g., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (e.g., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., chondrogenic cells). In some cases, iPSCs can be engineered to differentiate first into tissue-specific stem cells (e.g., mesenchymal stem cells, chondroprogenitor cells), which can be further induced to differentiate into committed cells (e.g., chondrogenic cells).

The term “embryonic stem cell” (ESCs) generally refers to cells derived from the naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiate directly into committed cells (e.g., chondrogenic cells). In some cases, ESCs can be engineered to differentiate first into tissue-specific stem cells (e.g., mesodermal stem cells), which can be further induced to differentiate into committed cells (e.g., chondrogenic cells).

The term “isolated stem cells” generally refers to any type of stem cells disclosed herein (e.g., ESCs, HSCs, mesenchymal or mesodermal stem cells (MSCs), etc.) that are isolated from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The term “chondrogenic” when applied to a cell or a population generally refers to the capacity of that cell or population to produce cartilage or to stimulate cartilage growth, under appropriate circumstances (e.g., in vivo or ex vivo conditions). The term “chondrogenic cell” generally refers to a cell that comprises tissue that produces cartilage or cartilage-related proteins. Non-limiting examples of chondrogenic cells are chondroblasts, chondrocytes, and cells which themselves differentiate into chondroblasts or chondrocytes, such as chondrogenic precursor cells. Chondrogenic cells develop from mesodermal stem cells (MSCs), including but not limited to paraxial mesoderm cells, sclerotome cells, and chondroprogenitor cells.

The terms “chondroprogenitor cells,” “chondrogenic precursor cells,” or “chondrogenic progenitor cells” as used interchangeably herein generally refer to chondrogenic-specific progenitor cells that are more committed than stem cells (e.g., ESCs, MSCs, iPSCs, etc.) but less differentiated than chondroblasts or chondrocytes. Chondroprogenitor cells can be generated ex vivo by engineering isolated stem cells. Chondroprogenitor cells can be generated in vivo by engineered stem cells in vivo, e.g., by administering such stem cells with any one of the heterologous genetic circuits disclosed herein.

### Overview

Biological programming, such as cellular programming, allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control the cell fate of a cell or plurality of cells by inducing differentiation or dedifferentiation and converting from one cell type to another. Cellular programming is controlled through the regulation of desired expression and/or activity levels of a plurality of genes in a cell.

Although CRISPR/Cas systems are widely used for gene editing, Cas is essentially a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this simple barcoding, often using exogenous fluorophores, doesn't allow for the regulation of endogenous genes to effect cell differentiation.

Further, differentiation or dedifferentiation of cells is currently enabled through the use of exogenous serums and growth factors which bypass the underlying machineries of a cell's programming. The use of exogenous serums, growth factors, and other similar methods results in cells that are instructed to differentiate, but which lack the concomitant underlying biology (e.g., chromatin in the correct state, etc.) This lack often results in cells that either undergo premature termination of differentiation into non-desired cell types or which undergo inefficient differentiation whereby there are low yields of target cell types, or the resulting desired differentiated cells are only semi-functional. Semi-functional cells may resemble cell types of interest, but may lack key biological features necessary for the normal function of the desired differentiated cell type.

Thus, there remains an unmet need for an activatable, CRISPR/Cas system and use of the same to edit a target polynucleotide (e.g., a genome of a cell, in particular a eukaryotic cell), using cascades of gRNAs to form genetic circuits in order to single-handedly affect gene regulation and, in turn, cell-fate determination without the use of serums and exogenous growth factors. The preprogrammed, activatable, and self-regulating gRNA cascade CRISPR/Cas system finds use, e.g., in gene therapy, genetic circuitry, and/or complex cell-fate determination and/or control.

The present disclosure provides systems and methods for engineering a CRISPR/Cas9 system, which includes a Cas endonuclease and an array of cognate single guide RNAs (sgRNA or gRNA) that harbor inactivation sequences in a non-essential region and are activatable, to allow for modulation and modification of that system without the need for serum, growth factors, or other additional exogenous signals. The present disclosure also provides for an engineered cell that can contain any of the above-mentioned systems or that can be capable of performing any of the above-mentioned methods.

### Systems and Methods for Cell Programming Towards Chondrogenic Lineage

Various aspects of the present disclosure provide systems for inducing a desired conversion from one type of cells into another type of cells. To this end, various aspects of the present disclosure provide methods for inducing a desired expression and/or activity levels (or profiles thereof) of one or more target genes in a cell.

In an aspect, the present disclosure provides for a system that converts a plurality of cells of a first type into a plurality of cells of a second cell type. The system can comprise a heterologous genetic circuit comprising a plurality of gate units. The plurality of gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s). The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). The plurality of gate units can be different (e.g., comprising different polynucleotide sequences). Each gate unit of the plurality of gate units can affect the modulation of the expression and/or activity levels of a distinct target gene or a plurality of distinct target genes.

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel sequential paths), or a combination thereof.

A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g. cell proliferation assays or metabolic activity assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

A plurality of gate units as disclosed herein can be sufficient to effect the conversion of a plurality of cells of a first cell type into a plurality of cells of a second cell type. For example, a plurality of gate units as disclosed herein can be sufficient to effect the conversion from a plurality of pluripotent stem cells (PSCs) into a plurality of tissue-specific progenitor cells. Alternatively, a plurality of gate units as disclosed herein can be necessary but insufficient to effect the conversion of a plurality of cells of a first cell type into a plurality of cells of a second cell type.

The outcome in the cell can comprise regulation of a distinct target gene or set of distinct target genes. The plurality of gate units can induce distinct modulations of the plurality of target genes (e.g., in a sequential manner), such that a collection of the modulations of the genes in concert yield a final expression and/or activity profile of the cell. The final expression and/or activity level profile of the cell can exemplify an outcome, such as a conversion of the cell from one cell type to another (or a process thereof).

In some cases, of the plurality of gate units, as disclosed herein, can be necessary but individually insufficient to effect the desired expression and/or activity profile of the target cell. Thus, the outcome in the cell (e.g., enhanced cell function, induced cell state, etc.) induced by the plurality of gate units may not be possible in absence of any one of the plurality of gate units. Alternatively, a degree or measure of the outcome in the cell induced by the plurality of gate units can be different (e.g., greater for a positive marker, or less for a negative marker) than a degree or measure of the outcome in a control cell that is induced by none, one or more, but not all of the plurality of gate units, and/or by all of the plurality gate units occurring through a different sequential order of events.

A second gate unit can be activated by a first gate unit (e.g. directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.). Yet in another alternative, the first gate unit and the second gate unit can be activated by different activating moieties (e.g., different polynucleotide molecules, such as different guide nucleic acid molecules).

In some cases, a first gate unit modulates a first target gene. Alternatively, or in addition to, a first gate unit can also modulate a second gate unit. The modulation of the second gate unit can occur at least or up to about 1 millisecond, about 2 milliseconds, about 3 milliseconds, about 4 milliseconds, about 5 milliseconds, about 6 milliseconds, about 7 milliseconds, about 8 milliseconds, about 9 milliseconds, about 10 milliseconds, about 20 milliseconds, about 30 milliseconds, about 40 milliseconds, about 50 milliseconds, about 60 milliseconds, about 70 milliseconds, about 80 milliseconds, about 90 milliseconds, about 100 milliseconds, about 200 milliseconds, about 300 milliseconds, about 400 milliseconds, about 500 milliseconds, about 600 milliseconds, about 700 milliseconds, about 800 milliseconds, about 900 milliseconds, about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 12 hours, about 16 hours, about 20 hours, about 24 hours, or more after the modulation of the first gate unit, as ascertained by rt-qPCR, Western blotting, or other methods.

In some cases, the second gate unit can modulate a second target gene. The modulation of the second target gene can occur at least or up to about 1 millisecond, about 2 milliseconds, about 3 milliseconds, about 4 milliseconds, about 5 milliseconds, about 6 milliseconds, about 7 milliseconds, about 8 milliseconds, about 9 milliseconds, about 10 milliseconds, about 20 milliseconds, about 30 milliseconds, about 40 milliseconds, about 50 milliseconds, about 60 milliseconds, about 70 milliseconds, about 80 milliseconds, about 90 milliseconds, about 100 milliseconds, about 200 milliseconds, about 300 milliseconds, about 400 milliseconds, about 500 milliseconds, about 600 milliseconds, about 700 milliseconds, about 800 milliseconds, about 900 milliseconds, about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 12 hours, about 16 hours, about 20 hours, about 24 hours, or more after the modulation of the first target gene, as ascertained by rt-qPCR, Western blotting, or other methods.

In some cases, the term “proGuide” as generally used herein may refer to such vector (e.g., a plasmid) that encodes the activatable gNA. The proGuide can be an example of a gate moiety. The proGuide can be an example of a gene regulating moiety.

A proGuide as provided herein can encode an activatable guide nucleic acid molecule, e.g., having the inactivation polynucleotide sequence (e.g., one or more polyX sequences, such as one or more polyT sequences). In some embodiments, a portion of the proGuide encoding the activatable guide nucleic acid molecule can comprise various regions that are sequentially linked, comprising a spacer sequence, an extra sequence (e,g, a linker sequence or a backbone sequence), an upstream stem, a poly T unit, and a downstream stem. In some embodiments, the extra sequence can exhibit at least 50%, at least 55%, at least 60%, at least 65%, at least 70%, at least 75%, at least 80%, at least 85%, at least 90%, at least 95%, at least 98%, at least 99%, or 100% sequence identity to GTTTTAGAGCTA (SEQ ID NO: 1935). In some embodiments, a portion of the proGuide encoding the activatable guide nucleic acid molecule can comprise various regions that are sequentially linked, comprising a spacer sequence, an extra sequence (e,g, a linker sequence or a backbone sequence), an upstream stem, a poly T unit, and a downstream stem, as shown in TABLE 1 (SEQ ID Nos: 1-1932). In some cases, upon modification or removal of the polyT unit, the upstream stem and the downstream stem can form a part of a scaffold sequence of a functional guide nucleic acid molecule. In some embodiments, a portion of the proGuide encoding the activatable guide nucleic acid molecule can comprise various regions that are sequentially linked, comprising a spacer sequence, an extra sequence (e,g, a linker sequence or a backbone sequence), an upstream stem, a poly T unit, and a downstream stem, as shown in TABLE 1 (SEQ ID Nos: 1-1932) as a concatenated sequence with “-” to distinguish between the various regions. In some cases, upon modification or removal of the polyT unit, the upstream stem and the downstream stem can form a part of a scaffold sequence of a functional guide nucleic acid molecule. In some embodiments, when a spacer sequence does not start with a G, a G is added before the spacer sequence. In some embodiments, the addition of the G before the spacer sequence helps increase the expression of the RNA moiety from a promotor. In some embodiments, stem 1 and stem2 are reverse complements of each other. In some embodiments, upstream stem and downstream stem are reverse complements of each other. In some embodiments, stem 1 can exhibit at least 50%, at least 55%, at least 60%, at least 65%, at least 70%, at least 75%, at least 80%, at least 85%, at least 90%, at least 95%, at least 98%, at least 99%, or 100% sequence identity to a member of the polynucleotide sequences as shown in Table 3 (SEQ ID Nos: 2024-2046). In some embodiments, stem2 can exhibit at least 50%, at least 55%, at least 60%, at least 65%, at least 70%, at least 75%, at least 80%, at least 85%, at least 90%, at least 95%, at least 98%, at least 99%, or 100% sequence identity to a member of the polynucleotide sequences as shown in Table 3 (SEQ ID Nos: 2047-2069).

In some embodiments, a proGuide as provided herein can encode an activatable guide nucleic acid molecule, e.g., having the inactivation polynucleotide sequence (e.g., one or more poly X sequences, such as one or more polyT sequences). In some cases, a portion of the proGuide encoding the activatable guide nucleic acid molecule can comprise various regions that are sequentially linked (e.g., from 5′ to 3′), comprising upstream stem (e.g., an upstream cut site), a poly T unit (or “proUnit” or “proGuide Unit” as used interchangeably herein), and a downstream stem (e.g., a downstream cut site). The upstream stem and the downstream stem may correspond to the “stem region” polynucleotide sequences that are at least partially complementary to each other.

A domain of the polynucleotide sequence that encodes (or corresponds to) the molecule of interest can comprise a polyX sequence. The polyX sequence can be sufficient to reduce expression of the molecule of interest (e.g., the guide nucleic acid molecule) from the polynucleotide sequence. For example, the polyX sequence can be disposed within the domain encoding the molecule of interest (e.g., not at either the 5′ end or the 3′ end of such domain), such that expression of the molecule of interest (e.g., transcription of an RNA molecule of interest) would be disrupted (e.g., terminated) in the middle of the expression.

Accordingly, the polyX sequence (e.g., in the polynucleotide sequence encoding the molecule of interest) may be referred to as a termination sequence (e.g., a non-canonical termination sequence for its sequence and/or its position), as a disruption sequence (e.g., for disruption of full expression of the molecule of interest), as an inactivation sequence (e.g., for inactivating function of the polynucleotide sequence or the molecule of interest).

In some cases, the non-canonical termination sequence can comprise or consist substantially of a polynucleotide sequence exhibiting at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the polynucleotide sequence of TTTTTTTTcagccaactccaaTTTTTTTT (SEQ ID NO: 1934), or a complementary sequence thereof.

In some cases, the poly X sequence can be located within (e.g., not at a terminal end) a polynucleotide sequence, such as a DNA sequence or an RNA sequence. In some cases, the poly X sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 3′ end of the polynucleotide sequence. In some cases, the poly X sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 5′ end of the polynucleotide sequence. In some cases, the polyX sequence can be located at a terminal end of a nucleic acid sequence.

In some cases, the polyT or polyU sequence can be located within (e.g., not at a terminal end) a polynucleotide sequence, such as a DNA sequence or an RNA sequence. In some cases, the polyT or polyU sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 3′ end of the polynucleotide sequence. In some cases, the polyT or polyU sequence can be located at least about 10, at least about 11, at least about 12, at least about 13, at least about 14, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 bases away from the 5′ end of the polynucleotide sequence. In some cases, the polyT or polyU sequence can be located at a terminal end of a nucleic acid sequence. In some cases, an RNA which comprises a polyU sequence can also be represented by a DNA which comprises a polyT sequence.

A poly X sequence (e.g., a polyT sequence or a polyU sequence) can comprise at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 30, at least about 40, at least about 50 X, at least about 60, at least about 70, at least about 80, at least about 90, or at least about 100 bases. A polyX sequence can comprise at most about 100, at most about 90, at most about 80, at most about 70, at most about 60, at most about 50, at most about 40, at most about 30, at most about 20, at most about 15, at most about 14, at most about 13, at most about 12, at most about 11, at most about 10, at most about 9, at most about 8, at most about 7, at most about 6, at most about 5, at most about 4, at most about 3, or at most about 2 X bases. A polyX sequence can be represented by a complementary poly X sequence in a corresponding complementary DNA strand (e.g., a polyT, as disclosed herein as a DNA sequence, can also be referred to as polyA in the complementary DNA strand). The polyX sequence as disclosed can comprise a plurality of X bases. The plurality of X bases can be disclosed sequentially adjacent to one another (e.g., TT, TTT, TTTT, TTTTT, etc.). Alternatively, or in addition to, the plurality of X bases can be separated by one or more additional nucleotides that are not X. The one or more additional nucleotides can comprise a single type of nucleotide or different types of nucleotides.

In some embodiments, a proGuide can comprise inactivation polynucleotide sequence that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to TTTTTTTTT or SEQ ID NO:1933, or a complementary sequence thereof.

In some embodiments, a proGuide can comprise a target polynucleotide domain at or adjacent to an inactivation polynucleotide sequence (e.g., at or adjacent to 5′ and/or 3′ ends of the inactivation polynucleotide sequences), which target polynucleotide domain can be targeted (e.g., via sequential activation mechanism of the heterologous genetic circuit as provided herein) to modify (e.g., edit, cleave) the inactivation polynucleotide sequence, thereby rendering the proGuide to express an activated guide nucleic acid molecule. The target polynucleotide domain of a proGuide may not exhibit sequence identity to any comparable endogenous polynucleotide sequence in a cell, thereby to avoid inadvertent targeting and modulation of an endogenous target gene.

In some embodiments, the inactivation polynucleotide sequence of the proGuide can be disposed between two target polynucleotide domains, which may or may not be targetable by a common guide nucleic acid sequence. In some cases, the two target polynucleotide domains can be reverse and complementary to one another, such that the inactivation polynucleotide sequence can be modified or cleaved by the same mechanism (e.g., same spacer sequence of a guide nucleic acid molecule).

In some embodiments, a proGuide can comprise a polynucleotide sequence that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members from SEQ ID NOs: 1-92 (e.g., ACAN targeting), SEQ ID NOs: 93-184 (e.g., COL2A1 targeting), SEQ ID NOs: 185-276 (e.g., MEF2D targeting), SEQ ID NOs: 277-460 (e.g., pax9 targeting), SEQ ID NOs: 461-644 (e.g., RUNX2 targeting), SEQ ID NOs: 645-920 (e.g., SOX5 targeting), SEQ ID NOs: 921-1288 (e.g., SOX6 targeting), SEQ ID NOs: 1289-1380 (e.g., SOX8 targeting), SEQ ID NOs: 1381-1472 (e.g., SOX9 targeting), SEQ ID NOs: 1473-1564 (e.g., SP1 targeting), SEQ ID NOs: 1565-1656 (e.g., TCF15 targeting), SEQ ID NOs: 1657-1748 (e.g., TCF7L2 targeting), SEQ ID NOs: 1749-1840 (e.g., TWIST1 targeting), SEQ ID NOs: 1841-1932 (e.g., UNCX targeting), or a complementary sequence thereof.

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g., a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively, or in addition to, inactivation can be achieved through the use of a transcriptional modulator system (e.g. a transcriptional repressor). Alternatively, or in addition to, inactivation can be achieved through CRISPRi steric hindrance without the necessity of an additional transcriptional modulator. An endonuclease-transcriptional modulator system (e.g., a Cas-repressor) can be used to achieve polynucleotide cleavage (e.g. for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively or in addition to, the endonuclease-transcriptional modulator system (e.g., a Cas-repressor) can be used to modulate target gene expression. Alternatively, or in addition to, a CAS transcriptional modulator system that lacks endonuclease activity (dCAS or bare CAS with a shortened spacer insufficient to support cleavage) targets to a DNA region and physically halts transcription elongation resulting in repression of the target gene (CRISPRi).

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g., a gate moiety and/or a gene regulating moiety of the first gate unit) that is responsible for inducing the first distinct modulation of the target gene.

The heterologous genetic circuit can comprise a plurality of gate units that are sequentially activated, e.g., activated in series one after another. The plurality of gate units can comprise a functional gate unit that is preconfigured such that it is activated to regulate (e.g., directly regulate) expression and/or epigenetic profile of a target gene (e.g., an endogenous targe gene). The plurality of gate units can further comprise one or more additional gate units that are preconfigured (i) to be activated prior to the functional gate unit and (ii) to effect a subsequent activation of the functional gate unit. In some cases, the one or more additional gate units can be preconfigured to be activated to regulate one or more additional target genes. Alternatively, the one or more additional gate units may not be preconfigured to regulate any target gene (e.g., any endogenous target gene) when activated. Such one or more additional gate units may instead serve to delay (e.g., in terms of time) activation of the functional gate unit during operation of the heterologous genetic circuit, thereby delaying the expression and/or epigenetic profile of the target gene of the functional gate unit, and thus the one or more additional gate units may be referred to as “blank” gate unit(s). The heterologous genetic circuit can comprise at least or up to about 1 blank gate unit, at least or up to about 2 blank gate units, at least or up to about 3 blank gate units, at least or up to about 4 blank gate units, at least or up to about 5 blank gate units, at least or up to about 6 blank gate units, at least or up to about 7 blank gate units, at least or up to about 8 blank gate units, at least or up to about 9 blank gate units, at least or up to about 10 blank gate units, at least or up to about 11 blank gate units, at least or up to about 12 blank gate units, at least or up to about 13 blank gate units, at least or up to about 14 blank gate units, at least or up to about 15 blank gate units, at least or up to about 16 blank gate units, at least or up to about 27 blank gate units, at least or up to about 18 blank gate units, at least or up to about 19 blank gate units, at least or up to about 20 blank gate units, at least or up to about 25 blank gate units, at least or up to about 30 blank gate units, at least or up to about 35 blank gate units, at least or up to about 40 blank gate units, at least or up to about 45 blank gate units, at least or up to about 50 blank gate units.

In some cases, use of the one or more blank gate units can delay activation of the functional gate unit (e.g., as ascertained by measurement of expression/epigenetic profile of the target gene, or as ascertained by measurement of expression of a functional variant or transcribed product of the functional gate unit) by at least or up to about 1 minute, at least or up to about 5 minutes, at least or up to about 10 minutes, at least or up to about 30 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 13 hours, at least or up to about 14 hours, at least or up to about 15 hours, at least or up to about 16 hours, at least or up to about 17 hours, at least or up to about 18 hours, at least or up to about 19 hours, at least or up to about 20 hours, at least or up to about 21 hours, at least or up to about 22 hours, at least or up to about 23 hours, at least or up to about 24 hours, at least or up to about 2 days, at least or up to about 3 days at least or up to about 4 days at least or up to about 5 days at least or up to about 6 days, or at least or up to about 7 days.

In some cases, modification of a target gene by a gate unit can inactivate a gene. For example, modification of a gene can stop expression and/or activity level of a target gene. Alternatively, modification of a gene can decrease the expression and/or activity level of a target gene. In some cases, modification of a gene can increase the expression and/or activity level of a target gene. Alternatively, modification of a gene can maintain the expression and/or activity level of a target gene.

In some cases, modification of a gene can decrease the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a gene can decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a gene can increase the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a gene can increase in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a gene can decrease the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a gene can decrease the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

In some cases, modification of a gene can increase the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a gene can increase the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

An expression and/or activity profile of a gene of interest (e.g. a differentiation marker) can be compared to a control gene (e.g., a house keeping gene such as GAPDH), relative expression levels of two or more genes of interest (e.g., a ratio of expression or activity level between a stem cell marker and a differentiation marker), relative average expression levels of a gene of interest compared to average expression levels of that same gene of interest in a cell type of interest, etc.

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least or up to about 1 gate moiety, at least or up to about 2 gate moieties, at least or up to about 3 gate moieties, at least or up to about 4 gate moieties, at least or up to about 5 gate moieties, etc.) and/or a gene regulating moiety (e.g., at least or up to about 1 gene regulating moiety, at least or up to about 2 gene regulating moieties, at least or up to about 3 gene regulating moieties, at least or up to about 4 gene regulating moieties, at least or up to about 5 gene regulating moieties, at least or up to about 6 gene regulating moieties, at least or up to about 7 gene regulating moieties, at least or up to about 8 gene regulating moieties, at least or up to about 9 gene regulating moieties, at least or up to about 10 gene regulating moieties, etc.). A gate moiety as disclosed herein can comprise a guide nucleic acid molecule (gNA) (e.g., at least or up to about 1 gNA molecule, at least or up to about 2 gNA molecules, at least or up to about 3 gNA molecules, at least or up to about 4 gNA molecules, at least or up to about 5 gNA molecules, etc.). A gene regulating moiety as disclosed herein can comprise a gNA (e.g., at least or up to about 1 gNA molecule, at least or up to about 2 gNA molecules, at least or up to about 3 gNA molecules, at least or up to about 4 gNA molecules, at least or up to about 5 gNA molecules, etc.). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least about 1, at least about 2, at least about 3, at least about 4, at least about 5, at least about 6, at least about 7, at least about 8, at least about 9, at least about 10, or more single-stranded breaks. In some cases, inactivation of a gene can be caused by at most about 10, at most about 9, at most about 8, at most about 7, at most about 6, at most about 5, at most about 4, at most about 3, at most about 2, or at most about 1 single-stranded breaks.

In some cases, a gNA can have a size (e.g., including both spacer sequence and scaffold sequence) of at least or up to about 60 nucleotides, at least or up to about 70 nucleotides, at least or up to about 80 nucleotides, at least or up to about 85 nucleotides, at least or up to about 90 nucleotides, at least or up to about 95 nucleotides, at least or up to about 100 nucleotides, at least or up to about 105 nucleotides, at least or up to about 110 nucleotides, at least or up to about 120 nucleotides, at least or up to about 130 nucleotides, at least or up to about 140 nucleotides, at least or up to about 150 nucleotides, or at least or up to about 200 nucleotides.

In some cases, a scaffold sequence of a gNA can have a size of at least or up to about 30 nucleotides, at least or up to about 35 nucleotides, at least or up to about 40 nucleotides, at least or up to about 45 nucleotides, at least or up to about 50 nucleotides, at least or up to about 55 nucleotides, at least or up to about 60 nucleotides, at least or up to about 65 nucleotides, at least or up to about 70 nucleotides, at least or up to about 75 nucleotides, at least or up to about 80 nucleotides, at least or up to about 85 nucleotides, at least or up to about 90 nucleotides, at least or up to about 95 nucleotides, at least or up to about 100 nucleotides, at least or up to about 100 nucleotides, at least or up to about 120 nucleotides, at least or up to about 130 nucleotides, at least or up to about 140 nucleotides, or at least or up to about 150 nucleotides.

In some cases, a spacer sequence of a gNA can have a size of at least or up to about 10 nucleotides, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30 nucleotides.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety may activate the initial gate unit without directly binding the at least the portion of the initial gate unit (e.g., through the use of electromagnetic energy). In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, a vector (or expression cassette) encoding the activatable gNA can comprise an inactivation polynucleotide sequence to render the gNA inactive until activated (e.g., until the inactivation polynucleotide sequence is modified or removed from the vector. For example, the inactivation polynucleotide sequence can encode a self-cleaving polynucleotide molecule (e.g., a ribozyme). Alternatively or in addition to, the inactivation polynucleotide sequence can encode non-canonical transcription termination sequence, as described below. The inactivation polynucleotide sequence can be a part of or adjacent to a region of the vector that encodes (i) a spacer sequence of the gNA, (ii) a scaffold sequence of the gNA, and/or (ii) any linker sequence between the spacer sequence and the scaffold sequence. The vector can comprise at least or up to about 1 inactivation polynucleotide sequence, at least or up to about 2 inactivation polynucleotide sequences, at least or up to about 3 inactivation polynucleotide sequences, at least or up to about 4 inactivation polynucleotide sequences, at least or up to about 5 inactivation polynucleotide sequences, at least or up to about 6 inactivation polynucleotide sequences, at least or up to about 7 inactivation polynucleotide sequences, at least or up to about 8 inactivation polynucleotide sequences, at least or up to about 9 inactivation polynucleotide sequences, or at least or up to about 10 inactivation polynucleotide sequences.

In some cases, the term “proGuide” as generally used herein may refer to such vector (e.g., a plasmid) that encodes the activatable gNA. The proGuide can be an example of a gate moiety. The proGuide can be an example of a gene regulating moiety.

In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the activatable gNA molecule comprises a non-canonical transcription termination sequence (e.g., a polyX sequence, such as a polyU sequence or a polyT sequence), such that a functional gNA molecule is not expressed until a gene encoding the activatable gNA having the non-canonical transcription termination sequence can be modified (e.g., to remove some or all of the transcription termination sequence). Thus, in absence of the modification of the transcription termination sequence, a non-functional variant (e.g., a non-functional fragment) of the gNA may be expressed. In some embodiments, the gNA can be synthetic. In some embodiments, the gNA can have a fluorescent label attached.

In some cases, a size of the polyT sequence is greater than or equal to a threshold length, wherein the threshold length is sufficient to reduce expression of the guide nucleic acid molecule from the polynucleotide sequence. Accordingly, a plasmid (e.g., a gate moiety or a gene regulating moiety) can encode an inactivated gNA comprising the polyT sequence that is greater than or equal to the threshold length, and editing of such plasmid to reduce the length of the polyT to below the threshold length can permit expression of the gNA in its entirety without early termination, thereby activating the gNA. In some cases, the polyT sequence comprises at least 5 T. In some cases, the polyT sequence comprises at least 7 T. In some cases, the polyT sequence comprises at least 8 T. In some cases, the polyT sequence comprises at least 10 T. In some cases, the polyT sequence comprises between 5 T and 15 T. In some cases, the polyT sequence comprises one or more additional nucleotides that are not T.

In some cases, a gene regulating moiety (e.g., a guide nucleic acid and/or an endonuclease) can be configured to bind to a target polynucleotide sequence operatively coupled to a target gene in a cell. The target gene can comprise an encoding polynucleotide sequence that encodes a target nucleic acid molecule or a target protein. The target polynucleotide sequence can be a part of the encoding polynucleotide sequence. Alternatively, the target polynucleotide sequence may not be a part of the encoding polynucleotide sequence. For example, the target polynucleotide sequence can be upstream of the encoding polynucleotide sequence (e.g., part of a promoter of the encoding polynucleotide sequence, such as a transcription start site (TSS).

As provided herein, when the heterologous genetic circuit is activated to induce a plurality of distinct modulations of a target gene, as provided herein, the plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. Alternatively or in addition to, the distinct modulation of the target gene can be substantially the same (e.g., the same). The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety may activate the initial gate unit without directly binding the at least the portion of the initial gate unit (e.g., through the use of electromagnetic energy). In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the gNA can be synthetic. In some embodiments, the gNA can have a fluorescent label attached.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least about 1, at least about 2, at least about 3, at least about 4, at least about 5, at least about 6, at least about 7, at least about 8, at least about 9, at least about 10, or more single-stranded breaks. In some cases, inactivation of a gene can be caused by at most about 10, at most about 9, at most about 8, at most about 7, at most about 6, at most about 5, at most about 4, at most about 3, at most about 2, or at most about 1 single-stranded breaks.

In some cases, a gNA can have a size (e.g., including both spacer sequence and scaffold sequence) of at least or up to about 60 nucleotides, at least or up to about 70 nucleotides, at least or up to about 80 nucleotides, at least or up to about 85 nucleotides, at least or up to about 90 nucleotides, at least or up to about 95 nucleotides, at least or up to about 100 nucleotides, at least or up to about 105 nucleotides, at least or up to about 110 nucleotides, at least or up to about 120 nucleotides, at least or up to about 130 nucleotides, at least or up to about 140 nucleotides, at least or up to about 150 nucleotides, or at least or up to about 200 nucleotides.

In some cases, a scaffold sequence of a gNA can have a size of at least or up to about 30 nucleotides, at least or up to about 35 nucleotides, at least or up to about 40 nucleotides, at least or up to about 45 nucleotides, at least or up to about 50 nucleotides, at least or up to about 55 nucleotides, at least or up to about 60 nucleotides, at least or up to about 65 nucleotides, at least or up to about 70 nucleotides, at least or up to about 75 nucleotides, at least or up to about 80 nucleotides, at least or up to about 85 nucleotides, at least or up to about 90 nucleotides, at least or up to about 95 nucleotides, at least or up to about 100 nucleotides, at least or up to about 100 nucleotides, at least or up to about 120 nucleotides, at least or up to about 130 nucleotides, at least or up to about 140 nucleotides, or at least or up to about 150 nucleotides.

In some cases, a spacer sequence of a gNA can have a size of at least or up to about 10 nucleotides, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30 nucleotides.

In some embodiments of any one of the systems disclosed herein, the gNA of the gate moiety and/or the gene regulating moiety (e.g., a gNA encoded by the gate moiety and/or the gene regulating moiety) can comprise a spacer sequence. In some cases, the spacer sequence can be specific to the target gene. Alternatively, the spacer sequence can be agnostic to the target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g., a Cas or a Cas-transcriptional modulator fusion protein) at a target polynucleotide sequence. Alternatively or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g. a Cas-activator system, a Cas-repressor system), without cleavage of the target gene.

In some cases, modification of a polynucleotide sequence (e.g., as a component of a gate unit, such as a gate moiety) or a target gene can be caused by a double-stranded break wherein there is a discontinuity in both nucleotide strands. In some cases, a number of such double-stranded break (e.g., necessary for such modification) can be at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10.

In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein. Alternatively or in addition not, the gene regulating moiety can induce epigenetic modification (or epigenome modification) as provided herein.

In some cases, the modification of the polynucleotide sequence or the target gene, as provided herein, can inactivate the polynucleotide sequence or the target gene. For example, modification of the polynucleotide sequence or the target gene can repress or reduce expression and/or activity level of the polynucleotide sequence or the target gene. In some cases, the modification of the polynucleotide sequence or the target gene, as provided herein, can activate the polynucleotide sequence or the target gene. For example, modification of the polynucleotide sequence or the target gene can increase expression and/or activity level of the polynucleotide sequence or the target gene.

In some cases, the modification of the polynucleotide sequence or the target gene, as provided herein, can comprise decreasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, at least or up to about 99%, or about 100% (e.g., as compared to a control that, for example, lacks the modification).

In some cases, the modification of the polynucleotide sequence or the target gene, as provided herein, can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, or at least or up to about 100-fold (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 150%, at least or up to about 200%, at least or up to about 300%, at least or up to about 400%, or at least or up to about 500% (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, at least or up to about 100-fold, at least or up to about 200-fold, at least or up to about 300-fold, at least or up to about 400-fold, at least or up to about 500-fold, or at least or up to about 1,000-fold (e.g., as compared to a control that, for example, lacks the modification).

In some embodiments of any one of the systems disclosed herein, the gNA of the gate moiety and/or the gene regulating moiety (e.g., a gNA encoded by the gate moiety and/or the gene regulating moiety) can comprise a spacer sequence. In some cases, the spacer sequence can exhibit specific binding to a target gene (e.g., an endogenous target gene). Alternatively, the spacer sequence can be agnostic to the target gene, but rather can exhibit specific binding to a target polynucleotide sequence of another gate moiety or another gene regulating moiety. Example spacer sequences can be found in Table 2 (SEQ ID Nos: 1940-2023).

Non-limiting examples of the one or more target genes can comprise ACAN, COL2A1, MEF2D, pax9, RUNX2, SOX5, SOX6, SOX8, SOX9, SP1, TCF15, TCF7L2, TWIST1, and/or UNCX. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1940-1943 (e.g., ACAN targeting), SEQ ID NOs: 1944-1947 (e.g., COL2A1 targeting), SEQ ID NOs: 1948-1951 (e.g., MEF2D targeting), SEQ ID NOs: 1952-1955, 1956-1959 (e.g., pax9 targeting), SEQ ID NOs: 1960-1963, 1964-1967 (e.g., RUNX2 targeting), SEQ ID NOs: 1968-1971, 1972-1975, 1976-1979 (e.g., SOX5 targeting), SEQ ID NOs: 1980-1983, 1984-1987, 1988-1991, 1992-1995 (e.g., SOX6 targeting), SEQ ID NOs: 1996-1999 (e.g., SOX8 targeting), SEQ ID NOs: 2000-2003 (e.g., SOX9 targeting), SEQ ID NOs: 2004-2007 (e.g., SP1 targeting), SEQ ID NOs: 2008-2011 (e.g., TCF15 targeting), SEQ ID NOs: 2012-2015 (e.g., TCF7L2 targeting), SEQ ID NOs: 2016-2019 (e.g., TWIST1 targeting), SEQ ID NOs: 2020-2023 (e.g., UNCX targeting), or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1940-1943_(e.g., ACAN targeting), SEQ ID NOs: 1944-1947 (e.g., COL2A1 targeting), SEQ ID NOs: 1948-1951 (e.g., MEF2D targeting), SEQ ID NOs: 1952-1955, 1956-1959 (e.g., pax9 targeting), SEQ ID NOs: 1960-1963, 1964-1967 (e.g., RUNX2 targeting), SEQ ID NOs: 1968-1971, 1972-1975, 1976-1979 (e.g., SOX5 targeting), SEQ ID NOs: 1980-1983, 1984-1987, 1988-1991, 1992-1995 (e.g., SOX6 targeting), SEQ ID NOs: 1996-1999 (e.g., SOX8 targeting), SEQ ID NOs: 2000-2003 (e.g., SOX9 targeting), SEQ ID NOs: 2004-2007 (e.g., SP1 targeting), SEQ ID NOs: 2008-2011 (e.g., TCF15 targeting), SEQ ID NOs: 2012-2015 (e.g., TCF7L2 targeting), SEQ ID NOs: 2016-2019 (e.g., TWIST1 targeting), SEQ ID NOs: 2020-2023 (e.g., UNCX targeting), or a complementary sequence thereof (e.g., with uracil-to-thymine conversion).

The gene regulating moiety as disclosed herein can comprise an endonuclease, such as a CRISPR-Cas protein exhibiting at least a portion of its nuclease activity. For example, the nuclease activity can be utilized to activate expression or activity of a guide nucleic acid molecule, thereby to activate at least a portion of the heterologous genetic circuit as described herein.

The gene regulating moiety as disclosed herein can comprise an endonuclease that is operatively coupled to a transcriptional effector, including a transcriptional activator or a transcriptional repressor, that is heterologous to the cell. The endonuclease may be naturally or may be engineered to exhibit reduced nuclease activity (or substantially no nuclease activity), such that the endonuclease can be used to specifically bind to a target gene, but without cleaving the target gene (e.g., endogenous gene, such as TBX, bHLH, SOX, collagen, etc.). In some cases, the nuclease can be a deactivated Cas (dCas). Instead, once the endonuclease identifies and binds the target gene, the transcriptional effector that is coupled (e.g., covalently or non-covalently coupled) to the endonuclease can interact with the target gene to either increase or decrease the expression level of the target gene, thereby either increasing or decreasing the activity level of the target gene. For example, the endonuclease and the transcriptional effector can be part of a fusion protein that is encoded by the same expression cassette.

FIG. 10 schematically illustrates use of the heterologous genetic circuit in conjunction with an endonuclease-transcriptional effector fusion (e.g., CRISPR Cas-transcriptional activator, such as Cas9-VPR). Each gate moiety can be a modified self-deactivating (e.g., self-destructing) guide RNA, which would be configured to form a complex with the CRISPR Cas-transcriptional effector fusion if not deactivated. The initial activating moiety (denoted as activating guide RNA or “aGuide”) can convert the first gate moiety to an activated guide RNA (denoted as a matureGuide). Each matureGuide can target an additional gene regulating moiety encoding an activatable guide RNA against a target gene (denoted as ramGuide), to activate such ranGuide. Subsequently, the activated ramGuide can form a complex with the CRISPR Cas-transcriptional effector fusion protein to bind the target gene and regulate expression level of the target gene. The activated matureGuide can also target an additional gate moiety that is downstream within the heterologous genetic circuit's signaling cascade, to subsequently regulate expression of one or more additional genes.

In some embodiments, the transcriptional effector can be a histone epigenetic modifier (or a histone modifier). In some cases, the histone epigenetic modifier can modulate histones through methylation (e.g., a histone methylation modifier, such as an amino acid methyltransferase, e.g., KRAB). In some cases, the histone epigenetic modifier can modulate histones through acetylation. In some cases, the histone epigenetic modifier can modulate histones through phosphorylation. In some cases, the histone epigenetic modifier can modulate histones through ADP-ribosylation. In some cases, the histone epigenetic modifier can modulate histones through glycosylation. In some cases, the histone epigenetic modifier can modulate histones through SUMOylation. In some cases, the histone epigenetic modifier can modulate histones through ubiquitination. In some cases, the histone epigenetic modifier can modulate histones by remodeling histone structure, e.g., via an ATP hydrolysis-dependent process.

In some embodiments, the transcriptional effector can be a gene epigenetic modifier (or a gene modifier). In some cases, a gene modifier can modulate genes through methylation (e.g., a gene methylation modifier, such as a DNA methyltransferase or DNMT). In some cases, a gene modifier can modulate genes through acetylation.

In some embodiments, the transcriptional effector can be derived from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, the transcriptional effector can be derived from a histone lysine methyltransferase. Non-limiting examples of histone lysine methyltransferases include EZH subfamily, Non-SET subfamily, Other SET subfamily, PRDM subfamily, SET1 subfamily, SET2 subfamily, SUV39 subfamily, SYMD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

Non-limiting examples of the transcriptional effector that enhances expression or activity of the target gene can include, but are not limited to, transcriptional activators such as VP16, VP64, VP48, VP160, p65 subdomain (e.g., from NFkB), vp64-p65-rta fusion protein (VPR), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZMYST3, MORFMYST4, SRC1, ACTR, PI 60, CLOCK; and DNA demethylases such as Ten-Eleven Translocation (TET) dioxygenase 1 (TETICD), TET1, DME, DML1, DML2, ROS1.

Non-limiting examples of the transcriptional effector that reduces expression or activity of the target gene can include, but are not limited to, transcriptional repressors such as the Kruppel associated box (KRAB or SKD); KOX1 repression domain; the Mad mSIN3 interaction domain (SID); the ERF repressor domain (ERD), the SRDX repression domain (e.g, for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARJD 1 A/RBP2, JARIDIB/PLU-1, JARID 1C/SMCX, JARIDID/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

Various aspects of the present disclosure provide for a plurality of heterologous genetic circuits that are individually activatable to modulate expression and/or activity levels of a plurality of distinct target genes in a sequential manner. In some embodiments, a first heterologous genetic circuit is activated to convert a plurality of cells from a first cell type to a second cell type and, subsequently, a second heterologous genetic circuit is activated to convert the plurality of cells from the second cell type to a target cell type.

In some embodiments, the activation of the second genetic circuit can be performed immediately following the activation of the first heterologous genetic circuit. Alternatively, the activation of the second genetic circuit can be performed at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 16 hours, at least about 20 hours, at least about 24 hours, at least about 2 days, at least about 3 days, at least about 4 days, at least about 5 days, at least about 6 days, at least about 7 days, at least about 2 weeks, at least about 3 weeks, at least about 4 weeks, at least about 2 months, at least about 3 months, at least about 4 months, at least about 5 months, at least about 6 months, at least about 1 year, or more subsequent to the activation of the first genetic circuit.

One or more target genes as disclosed herein can comprise one or more endogenous genes (e.g., genomic DNA, mRNA, mitochondrial DNA, etc.), exogenous genes, transgenes, or a combination thereof.

One or more target genes as disclosed herein can comprise a cell differentiation regulatory factor, a molecular function regulatory factor, a binding factor, a fusogenic factor, a protein folding chaperone, a protein tag, a RNA folding chaperone, a cell signaling factor, an immune response factor, a sensory receptor, a cell structural factor, a protein binding factor, a cargo receptor, a catalytic factor, or a small molecule sensor.

One or more target genes as disclosed herein can comprise a cell differentiation regulatory factor that comprises a growth factor, a transcription factor, a myogenic regulatory factor, an immune cell regulatory factor, a neuronal regulatory factor, a stem cell differentiation factor, a chondrogenic regulatory factor, an osteogenic regulatory factor, a senescence factor, a stemness factor (e.g., de-differentiation factor), etc.

In some cases, the one or more target genes (e.g., one or more chondrogenic regulatory factors) can comprise Sox2, Sox6, Sox9, Shox2, Gli3, Trps1, Oct4, NANOG, Chondrogenicx3.2, Brachyury, Mixl1, Tbx6, Msgn1, Parxis, Pax9, Runx2, Runx3, Smad1, Smad5, and Smad8.

In some cases, the one or more target genes can comprise a homeobox gene. Homeobox genes are genes that regulate, for example, large-scale anatomical features in the early stages of embryonic development. Types of homeobox genes include HOX genes, LIM genes, PAX genes, POU genes, CERS genes, HNF genes, SINE genes, CUT genes, ZF genes, paraHOX genes, DLX genes, TALE genes, PRD genes, and NKL genes. Non-limiting examples of homeobox genes can include HOXA1, HOXA2, HOXA3, HOXA4, HOXA5, HOXA6, HOXA7, HOXA9, HOXA10, HOXA11, HOXA13, HOXB1, HOXB2, HOXB3, HOXB4, HOXB5, HOXB6, HOXB7, HOXB8, HOXB9, HOXB13, HOXC4, HOXC5, HOXC6, HOXC8, HOXC9, HOXC10, HOXC11, HOXC12, HOXC13, HOXD1, HOXD3, HOXD4, HOXD8, HOXD9, HOXD10, HOXD11, HOXD12, HOXD13, CDX1, CDX2, CDX4, GSX1, GSX2, PDX1, EVX1, EVX2, GBX1, GBX2, MEOX1, MEOX2, MNX1, DLX1, DLX2, DLX3, DLX4, DLX5, DLX6, IRX1, IRX2, IRX3, IRX4, IRX5, IRX6, MEIS1, MEIS2, MEIS3, MKX, PBX1, PBX2, PBX3, PBX4, PKNOX1, PKNOX2, TGIF1, TGIF2, TGIF2LX, TGIF2LY, ISL1, ISL2, LHX1, LHX2, LHX3, LHX4, LHX5, LHX6, LHX8, LHX9, LMXIA, LMX1B, HDX, POUIF1, POU2F1, POU2F2, POU2F3, POU3F1, POU3F2, POU3F3, POU3F4, POU4F1, POU4F2, POU4F3, POU5F1, POU5F1P1, POU5F1P4, POU5F2, POU6F1, POU6F2, LASS2, LASS3, LASS4, LASS5, LASS6, HMBOX1, HNFIA, HNFIB, SIX1, SIX2, SIX3, SIX4, SIX5, SIX6, ONECUT1, ONECUT2, ONECUT3, CUX1, CUX2, SATB1, SATB2, ADNP, ADNP2, TSHZ1, TSHZ2, TSHZ3, ZEB1, ZEB2, ZFHX2, ZFHX3, ZFHX4, ZHX1, HOMEZ, ALX1 (CARTI), ALX3, ALX4, ARGFX, ARX, DMBX1, DPRX, DRGX, DUXA, DUXB, DUX (1, 2, 3, 4, 4c, 5), ESXI, GSC, GSC2, HESX1, HOPX, ISX, LEUTX, MIXL1, NOBOX, OTP, OTX1, OTX2, CRX, PAX2, PAX3, PAX4, PAX5, PAX6, PAX7, PAX8, PHOX2A, PHOX2B, PITX1, PITX2, PITX3, PROPI, PRRX1, PRRX2, RAX, RAX2, RHOXF1, RHOXF2/2B, SEBOX, SHOX, SHOX2, TPRX1, UNCX, VSX1, VSX2, BARHL1, BARHL2, BARX1, BARX2, BSX, DBX1, DBX2, EMX1, EMX2, EN1, EN2, HHEX, HLX1, LBX1, LBX2, MSX1, MSX2, NANOG, NOTO, TLX1, TLX2, TLX3, TSHZ1, TSHZ2, TSHZ3, VAX1, VAX2, VENTX, NKX2-1, NKX2-4, NKX2-2, NKX2-8, NKX3-1, NKX3-2, NKX2-3, NKX2-5, NKX2-6, HMX1, HMX2, HMX3, NKX6-1, NKX6-2, and NKX6-3.

In some cases, the target gene can comprise MIXL1. MIXL1 is a transcription factor that preferentially binds to the DNA sequence TAAT on the MIX gene and plays a role in mesoderm patterning and tissue specification at gastrulation.

In some cases, the target gene can comprise UNCX. UNCX is a transcription factor that is involved in somitogenesis and neurogenesis. UNCX is also required for the maintenance and differentiation of specific elements of the axial skeleton.

In some cases, the target gene can comprise PAX9. PAX9 is a transcription factor that is required for development of the thymus, parthyroid glands, ultimobranchial bodies, teeth, skeleton, and limbs.

In some cases, the one or more target genes can comprise T-box transcription factors (TBX genes). TBX transcription factors are involved in development. T-box proteins have relatively large DNA-binding domains. Non-limiting examples of TBX transcription factors can include TBX1, TBX2, TBX3, TBX4, TBX5, TBX6, TBX10, TBX15, TBX18, TBX19, TBX20, TBX21, TBX22, and TBXT (Brachyury protein).

In some cases, the target gene can comprise TBXT. TBXT, also known as T-box transcription factor T or brachyury protein, functions as a transcription factor in the T-box family of genes. TBXT has a role in defining the midline of a bilateral organism, helping to establish the anterior-posterior axis. It can also assist in defining the mesoderm during gastrulation.

In some cases, the target gene can comprise TBX6. TBX6, also known as T-box transcription factor 6, is involved in the segmentation of the paraxial mesoderm into somites.

In some cases, the one or more target genes can comprise a basic helix-loop-helix transcription factor (bHLH gene). bHLH transcription factors are involved in the regulation of the cell cycle and many other developmental processes. bHLH proteins have a basic helix-loop-helix protein structure. Non-limiting examples of bHLH transcription factors can include AHR, AHRR, ARNT, ARNT2, ARNTL, ARNTL2, ASCL1, ASCL2, ASCL3, ASCL4, ATOHI, ATOH7, ATOH8, BHLHB2, BHLHB3, BHLHB4, BHLHB5, BHLHB8, CLOCK, EPASI, FERD3L, FIGLA, HAND1, HAND2, HES1, HES2, HES3, HES4, HES5, HES6, HES7, HEY1, HEY2, HIFIA, ID1, ID2, ID3, ID4, KIAA2018, LYLI, MASHI, MATH2, MAX, MESP1, MESP2, MIST1, MITF, MLX, MLXIP, MLXIPL, MNT, MSC, MSGN1, MXDI, MXD3, MXD4, MXI1, MYC, MYCL1, MYCL2, MYCN, MYF5, MYF6, MYOD1, MYOG, NCOAI, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTFIA, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2.

In some cases, the target gene can comprise MSGN1. MSGN1, also known as mesogenin 1, is a Wnt-activated bHLH transcription factor which is involved in mesoderm formation and regulation of transcription by RNA polymerase II. MSGN1 can also be involved with somitogenesis.

In some cases, the target gene can comprise TCF15. TCF15 is an early transcription factor that plays a role in somitogenesis, paraxial mesoderm development, and regulation of stem cell pluripotency.

In some cases, the one or more target genes can comprise a SRY-related box transcription factor (SOX gene). SOX transcription factors are involved in developmental regulation. Non-limiting examples of SOX transcription factors can include SOX1, SOX2, SOX3, SOX4, SOX5, SOX6, SOX7, SOX8, SOX9, SOX10, SOX11, SOX12, SOX13, SOX14, SOX15, SOX17, SOX18, SOX21, SOX30, and SRY.

In some cases, the one or more target genes can comprise SOX group A, comprising SRY. In some cases, the one or more target genes can comprise SOX group B1, comprising SOX1, SOX2 and/or SOX3. In some cases, the one or more target genes can comprise SOX group B2, comprising SOX14 and/or SOX21. In some cases, the one or more target genes can comprise SOX group C, comprising SOX4, SOX11 and/or SOX12. In some cases, the one or more target genes can comprise SOX group D, comprising SOX5, SOX6, and/or SOX13. In some cases, the one or more target genes can comprise SOX group E, comprising SOX8, SOX9, and/or SOX10. In some cases, the one or more target genes can comprise SOX group F, comprising SOX7, SOX 17, and/or SOX18. In some cases, the one or more target genes can comprise SOX group G, comprising SOX15. In some cases, the one or more target genes can comprise SOX group H, comprising SOX30.

In some cases, the target gene can comprise SOX6. SOX6 is a transcription factor which is required for development of the central nervous system, chondrogenesis, and maintenance of cardiac and skeletal muscle cells.

In some cases, the target gene can comprise SOX9. SOX9 is a transcription factor which acts during chondrocyte differentiation and regulates transcription of the anti-Muellerian hormone (AMH) gene.

In some cases, the one or more target genes can comprise a collagen. Collagens are fibrous proteins and are the major elements of skin, bone, tendon, cartilage, blood vessels, and teeth. Collagens form insoluble fibers of high tensile strength. Non-limiting examples of collagen genes can include COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4P1, COL6A4P2, COL6A5, COL6A6, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, COL26A1, COL27A1, and COL28A1.

In some cases, the target gene can comprise COL2A1. COL2A1 is a component of the pro-alpha1 chain of type II collagen. Type II collagen adds structure and strength to connective tissues that support the body's muscles, joints, organs, and skin. Type II collagen is found primarily in cartilage.

In some cases, use of the heterologous genetic circuit as disclosed herein can be used to differentiate mesodermal stem cells (MSCs) into chondroprogenitor cells whereby at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% of the resulting cells generated by using the heterologous genetic circuit as disclosed herein are the target cell type.

In some cases, use of the heterologous genetic circuit as disclosed herein can be used to differentiate mesodermal stem cells (MSCs) into chondroprogenitor cells, e.g., in absence of one, two, or all of feeder cells, serum, and exogenous growth factors. Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹⁵, at least about 2×10¹⁵, at least about 5×10¹⁵, or more chondrogenic cells from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or more chondroprogenitor cells.

In some cases, use of the heterologous genetic circuit as disclosed herein can be used to differentiate pluripotent stem cells (PSCs, such as induced PSCs or iPSCs) into chondrogenic cells, e.g., in absence of one, two, or all of feeder cells, serum, and exogenous growth factors. Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹⁵, at least about 2×10¹⁵, at least about 5×10¹⁵, or more chondrogenic cells from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or more chondroprogenitor cells.

Such generation of chondrogenic cells by using the heterologous genetic circuit as disclosed herein can be achieved within the span of at most about 60 days, at most about 55 days, at most about 50 days, at most about 45 days, at most about 40 days, at most about 35 days, at most about 30 days, at most about 25 days, at most about 20 days, at most about 15 days, at most about 10 days, at most about 7 days, at most about 6 days, at most about 5 days, at most about 4 days, at most about 3 days, at most about 2 days, at most about 1 day, or less.

In some cases, the chondrogenic cells generated through this method produce more cartilage as compared to chondrogenic cells obtained via directed differentiation. The chondrogenic cells generated using the provided methods can produce at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 120%, at least about 150%, or at least about 200% more cartilage than chondrogenic cells obtained via directed differentiation. Alternatively, or in addition to, the chondrogenic cells generated through this method may have an equivalent amount of cartilage produced as compared to chondrogenic cells obtained via directed differentiation.

In some cases, the chondrogenic cells or chondroprogenitor cells generated through this method exhibit higher expression levels of two or more positive chondroprogenitor cell markers as compared to control chondroprogenitor cells. Non-limiting examples of positive chondroprogenitor cell markers can include CD146, CD73, CD112, and BMPR1.

In some cases, the chondrogenic cells or chondroprogenitor cells generated through this method exhibit at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000% higher expression levels of positive chondroprogenitor cell markers as compared to control chondroprogenitor cells.

In some cases, the chondrogenic cells or chondroprogenitor cells generated through this method exhibit at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold higher expression levels of positive chondroprogenitor cell markers as compared to control chondroprogenitor cells.

In some cases, the chondrogenic cells or chondroprogenitor cells generated through this method exhibit higher expression levels of two or more negative chondroprogenitor cell markers as compared to control chondroprogenitor cells. Non-limiting examples of negative chondroprogenitor cell markers can include CD326 and CD309.

In some cases, the chondrogenic cells or chondroprogenitor cells generated through this method exhibit at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000% lower expression levels of negative chondroprogenitor cell markers as compared to control chondroprogenitor cells.

In some cases, the chondrogenic cells or chondroprogenitor cells generated through this method exhibit at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold lower expression levels of negative chondroprogenitor cell markers as compared to control chondroprogenitor cells.

In some cases, expression levels of chondroprogenitor cell markers can be measured using methods such as, but not limited to, RT-PCR, Western blotting, Northern blotting, protein staining, mRNA staining, and RNA-sequencing.

In some cases, expression levels can be measured at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 28 hours, at least about 32 hours, at least about 36 hours, at least about 40 hours, at least about 44 hours, at least about 48 hours, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days or more days after the introduction of a genetic circuit.

In some cases, the chondrogenic cells or chondroprogenitor cells generated through this method can be chondrocytes. Alternatively, chondrogenic cells or chondroprogenitor cells generated through this method can be cells other than chondrocytes (e.g., chondroblasts). In some cases, the progenitor cells generated can be substantially mitotically dormant. Alternatively, the progenitor cells generated can be substantially mitotically active.

In some cases, a first gate unit can be configured to reduce expression and/or activity levels of one or more target genes. In some cases, a first gate unit can be configured to enhance expression and/or activity levels of one or more target genes. In some cases, a first gate unit can be configured to maintain expression and/or activity levels of one or more target genes.

In some cases, modulation of a first target gene can occur prior to modulation of a second target gene. In some cases, modulation of a first target gene can occur subsequent to modulation of a second target gene. In some cases, modulation of a first target gene can occur at about the same time as modulation of a second target gene.

In some cases, use of the heterologous genetic circuit can induce cells to differentiate into the cell type of interest in absence of growth factors, serum (fetal bovine serum, human serum AB, etc.), or other exogenous cell differentiation regulatory factors or mediums. Serum can comprise liquid fractions of clotted blood, including nutritional and macromolecular factors essential for cell growth.

Alternatively, use of the heterologous genetic circuit can induce cells to differentiate into the cell type of interest using a reduced amount of serum and/or growth factors (e.g., reduced by at least about 1%, at least about 2%, at least about 3%, at least about 4%, at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or substantially free of serum). Reduced amounts of serum can allow for more consistency across experiments or batches of cells, increased growth and/or productivity of differentiated cells, better control over physiological responsiveness, and reduced risk around contamination by serum-born agents in cell culture.

In some cases, use of the heterologous genetic circuit in the stem cells (e.g., iSPCs, MSCs) can induce the MSCs to differentiate into chondrogenic cells in absence of growth factors, serum (fetal bovine serum, human serum AB, etc.), or other exogenous cell differentiation regulatory factors or mediums. In some cases, use of the heterologous genetic circuit as disclosed herein can be used to differentiate the stem cells into chondrogenic cells in the absence of one or both growth factors and serum. The resulting chondrogenic cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population.

In some cases, conversion from one cell type (e.g., PSCs, MSCs or chondroprogenitor cells) to another cell type (e.g., chondrogenic cells) using a heterologous genetic circuit can result in the target cell type. Alternatively, conversion from one cell type (e.g., PSCs, MSCs or chondroprogenitor cells) to another cell type (e.g., chondrogenic cells) using a heterologous genetic circuit can result in an intermediate cell type. An intermediate cell type can undergo a second conversion using a second genetic circuit to result in the target cell type.

The conversion of a cell from one cell type to another can comprise the regulation of a plurality of target genes. For example, the conversion can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The conversion can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic circuit and/or (ii) the activating moiety of the heterologous genetic circuit.

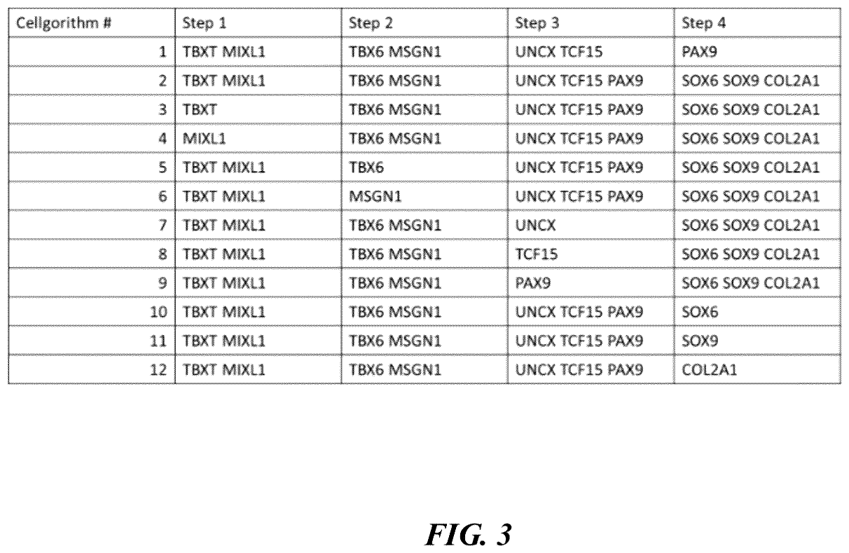

As demonstrated in FIG. 3, various heterologous genetic circuits can be designed to modulate expression or activity levels of a plurality of genes (e.g., a plurality of endogenous genes) in a cell over a plurality of different time points.

For example, a heterologous genetic circuit can be designed to (i) modulate expression levels of a first gene and (ii) subsequently modulate expression levels of a second gene. For example, a heterologous genetic circuit can be designed to (i) activate expression levels of a first gene and (ii) subsequently activate expression levels of a second gene. Alternatively, a heterologous genetic circuit can be designed to (i) activate expression levels of a first gene and (ii) subsequently reduce expression levels of a second gene. Alternatively, a heterologous genetic circuit can be designed to (i) reduce expression levels of a first gene and (ii) subsequently activate expression levels of a second gene. Alternatively, a heterologous genetic circuit can be designed to (i) reduce expression levels of a first gene and (ii) subsequently reduce expression levels of a second gene. The first gene and the second gene can be the same gene. Alternatively, the first gene and the second gene can be different genes. A heterologous genetic circuit can be designed to modulate the expression levels of an additional gene. A heterologous genetic circuit can be designed to include at least 1, at least 2, at least 3, at least 4, at least 5, at least 6, at least 7, at least 8, at least 9 at least 10, or more additional genes. An additional gene can be activated. Alternatively, an additional gene can be reduced. A heterologous genetic circuit can be designed to modulate expression of the additional gene prior to modulation of expression levels of both the first gene and the second gene. Alternatively or in addition to, a heterologous genetic circuit can be designed to modulate expression of the additional gene subsequent to modulation of expression levels of the first gene and prior to modulation of expression levels of the second gene. Alternatively or in addition to, a heterologous genetic circuit can be designed to modulate expression of the additional gene subsequent to modulation of expression levels of both the first gene and the second gene.

For example, a heterologous genetic circuit can be designed to (i) modulate expression levels of a first homeobox gene and (ii) subsequently modulate expression levels of a second homeobox gene. For example, a heterologous genetic circuit can be designed to (i) activate expression levels of a first homeobox gene and (ii) subsequently activate expression levels of a second homeobox gene. Alternatively, a heterologous genetic circuit can be designed to (i) activate expression levels of a first homeobox gene and (ii) subsequently reduce expression levels of a second homeobox gene. Alternatively, a heterologous genetic circuit can be designed to (i) reduce expression levels of a first homeobox gene and (ii) subsequently activate expression levels of a second homeobox gene. Alternatively, a heterologous genetic circuit can be designed to (i) reduce expression levels of a first homeobox gene and (ii) subsequently reduce expression levels of a second homeobox gene. The first homeobox gene and the second homeobox gene can be the same gene. Alternatively, the first homeobox gene and the second homeobox gene can be different genes.

A heterologous genetic circuit can be designed to include another gene in addition to the first and second homeobox genes. An additional gene can be modulated before, simultaneously with, or after the first homeobox protein. Alternatively or in addition to, an additional gene can be modulated before, simultaneously with, or after the second homeobox protein. The heterologous genetic circuit can be designed to activate the additional gene. Alternatively, the heterologous genetic circuit can be designed to reduce the additional gene. The additional gene can be, but is not limited to, a T-box transcription factor (TBX), a basic helix-loop-helix transcription factor (bHLH), a SOX, and a collagen.

A heterologous genetic circuit can be designed to include two members selected from the group consisting of a homeobox protein, a T-box transcription factor (TBX), and a basic helix-loop-helix transcription factor (bHLH). A heterologous genetic circuit can be designed to include a homeobox protein and a TBX. A heterologous genetic circuit can be designed to include a homeobox protein and a bHLH. A heterologous genetic circuit can be designed to include a TBX and a bHLH.

A heterologous genetic circuit can be designed to include a first member that is a homeobox protein, a TBX, or a bHLH and a second member that is a SOX or collagen. A heterologous genetic circuit can be designed to include a homeobox protein and a SOX. A heterologous genetic circuit can be designed to include homeobox protein and a collagen. A heterologous genetic circuit can be designed to include a TBX and a SOX. A heterologous genetic circuit can be designed to include a TBX and a collagen. A heterologous genetic circuit can be designed to include a bHLH and a SOX. A heterologous genetic circuit can be designed to include a bHLH and a collagen.

For example, heterologous genetic circuit #10 can be designed to (i) activate expression level of TBXT and MIXL1 at time point 1 (denoted as step 1), (ii) subsequently activate expression levels of MSGN1 and TBX6 at a time point after step 1 (denoted as step 2), (iii) subsequently activate expression levels of UNCX, TCF15, and PAX9 at a time point after step 2 (denoted as step 3), and (iv) subsequently activate expression levels of SOX6 at a time point after step 3 (denoted as step 4). For comparison, a control heterologous genetic circuit (denoted as All-at-once 1) can be designed to simultaneously activate the same target endogenous genes from the heterologous genetic circuit #10.

Activating a heterologous genetic circuit in a cell as disclosed herein can modulate expression or activity levels of a plurality of genes over a plurality of different time points, to effect conversion of the cell into a different cell type (e.g., stem cells into tissue-specific progenitor cells, etc.). A rate of such cell type conversion by use of the heterologous genetic circuit can be greater than a rate of the cell type conversion by use of the control heterologous genetic circuit (e.g., for simultaneous activation of multiple target genes) by at least or up to about 1 percent (%), at least or up to about 2%, at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 60%, at least or up to about 70%, at least or up to about 80%, at least or up to about 90%, or at least or up to about 95%.

Activating a heterologous genetic circuit in a cell as disclosed herein can modulate expression or activity levels of a plurality of genes over a plurality of different time points, to effect conversion of the cell into a different cell type (e.g., stem cells into tissue-specific progenitor cells, etc.). The conversion of the cell into a different cell type can occur in less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, less than about 8 days, less than about 7 days, less than about 6 days, less than about 5 days, less than about 4 days, less than about 3 days, less than about 2 days, or less than about 1 day.

A cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, an secretory cell, a blood cell, a germ cell, a nurse cell, a storage cell, an enteroendocrine cell, a pituitary cell, a mesoderm cell, a chondroprogenitor cell, a neurosecretory cell, a duct cell, an odontoblast, a cementoblast, a glial cell, or an interstitial cell.

Non-limiting examples of such cell can include lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); paraxial mesoderm cells cartilage cells, including sclerotome cells, Chondroblasts, or Chondrocytes, skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In an aspect, the present disclosure provides for systems and methods that can convert a plurality of pluripotent stem cells (PSCs) into a plurality of tissue-specific progenitor cells.

A pluripotent stem cell can comprise an induced pluripotent stem cell (iPSC), or an embryonic stem cell (ESC). A tissue-specific progenitor cells can comprise a mesenchymal stem cell (MSC), a hematopoietic stem cell (HSC), a myeloid progenitor cell, a muscle stem cell, a chondroprogenitor cell, a neural stem cell, an epithelial stem cell, an epidermal stem cell, a mammary stem cell, an intestinal stem cell, a neural crest stem cell, or a testicular stem cell.

Various aspects of the present disclosure provide engineered cells that are programmed to induce a desired expression and/or activity level (or profile thereof) of one or more target genes in a cell.

In some embodiments, the engineered cell (e.g., the engineered chondrogenic cell) of the present disclosure can be generated from an isolated stem cell (e.g., isolated MSCs or iPSCs). The heterologous genetic circuit and/or its components (e.g. gate units, gate moieties, activating moieties, etc.), as disclosed herein, can be introduced during any stage (or cellular state) between and including (a) the isolated stem cell and (b) the differentiated chondrogenic cell state thereof (e.g. a terminally differentiated chondrogenic cell state, such as a chondrocyte.

The engineered cell (e.g., the engineered chondrogenic cell) of the present disclosure can be used (e.g., administered) to treat a subject in need thereof. The subject can have or can be suspected of having a condition, such as a disease (e.g., cancer). A cell (e.g., a stem cell or a differentiated cell) can be obtained from the subject and such cell can be cultured ex vivo and genetically modified to generate any subject engineered cell (e.g. a chondrogenic cell) as disclosed herein. Subsequently, the engineered immune cell can be administered to the subject for adaptive immunotherapy. Thus, the engineered cell can be autologous to the subject in need thereof. Alternatively, the engineered cell can be allogeneic to the subject (e.g., allogeneic stem cell transplantation, allogeneic adoptive immunotherapy, etc.).

The engineered cells as disclosed herein can be administered to the subject prior to, concurrently with, or subsequent to activation of the heterologous genetic circuit(s) in the engineered stem cells. For example, the engineered cells can be activated subsequent to being administered into the subject, e.g., by administering to the subject an activator of the heterologous genetic circuit(s).

The subject can be treated (e.g., administered with) a population of engineered cells (e.g., engineered muscle cells) of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses. Alternatively, or in addition to, the subject can be treated (e.g., administered with) a population of engineered cells (e.g., engineered T cells) of the present disclosure for at least about 1 week, at least about 2 weeks, at least about 3 weeks, at least about 4 weeks, at least about 5 weeks, at least about 6 weeks, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, at least about 30 years, at least about 40 years, at least about 50 years, at least about 60 years, at least about 70 years, at least about 80 years, at least about 90 years, or at least about 100 years.

Any one of the methods disclosed herein can be utilized to treat a target cell, a target tissue, a target condition, or a target disease of a subject.

The target disease of a subject can be a disease which affects the cartilage. A disease which affects cartilage can include, but is not limited to, arthritis (e.g., osteoarthritis, rheumatoid arthritis, juvenile idiopathic arthritis), gout, systemic lupus erythematosus, seronegative spondyloarthropathies, costochondritis, herniation, achondroplasia, or polychondritis. The target condition of a subject can be injury (e.g., a joint injury such as a knee injury).

The target disease of the subject can be cancer or tumor. Non-limiting examples of cancer can include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

Non-limiting examples of the target tissue can include cells, for example chondrogenic cells, can be obtained from a subject. Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

The present disclosure also provides a composition comprising the engineered genetic circuit(s) as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered cells. Alternatively or in addition to, the activator(s) can be in a different and separate composition from the engineered cells.

In some cases, the engineered progenitor cells disclosed herein can exhibit (i) comparable or enhanced regenerative capacity; (ii) comparable or enhanced in vitro expression; (iii) comparable or enhanced genetic editing capabilities; (iv) comparable or enhanced immunotolerance; (v) comparable or shorter manufacturing timelines; (vi) comparable or fewer growth factor or culturing requirements; and/or (vii) comparable or enhanced safety, as compared to a control progenitor cell.

The control progenitor cell can be generated by any method comprising expansion of a progenitor cell (e.g., chondroprogenitor cell) isolated from a tissue, directed iPSC differentiation (e.g., using exogenous growth factors), and/or transgenic iPSC differentiation (e.g., viral transduction of heterologous genes).

In some cases, the tissue-specific progenitor cells can be stored in a receptacle (e.g., a sterilized vial). In some cases, the tissue-specific progenitor cells are stored at a temperature of at most about 10° C., at most about 5° C., at most about 4° C., at most about 0° C., at most about −5° C., at most about −10° C., at most about −20° C., at most about −30° C., at most about −40° C., at most about-50° C., at most about −60° C., at most about −70° C., at most about −80° C., at most about −90° C., at most about −100° C., at most about −110° C., at most about −120° C., at most about −130° C., at most about −140° C., at most about −150° C., at most about −160° C., at most about −170° C., at most about −180° C., at most about −190° C., at most about −200° C., or colder.

### Pharmaceutical Compositions

In some cases, methods disclosed herein comprise administering at least one of the tissue-specific progenitor cells to a subject in need thereof. A subject can be an animal. A subject can be a mammal (e.g., a primate, a horse, a cat, a dog, a cow, a pig, a sheep, a goat, a mouse, a rabbit, a rat, a guinea pig). A subject can be a human subject.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

### EXAMPLES

### Example 1: Differentiation of Chondrogenic Progenitor Cells

Tissue-specific cells (e.g., chondroprogenitor cells) can be generated from less-differentiated cells (e.g., stem cells, such as iPSCs) by the systems and methods of the present disclosure.

### A. Generation of Chondroprogenitor Cells.

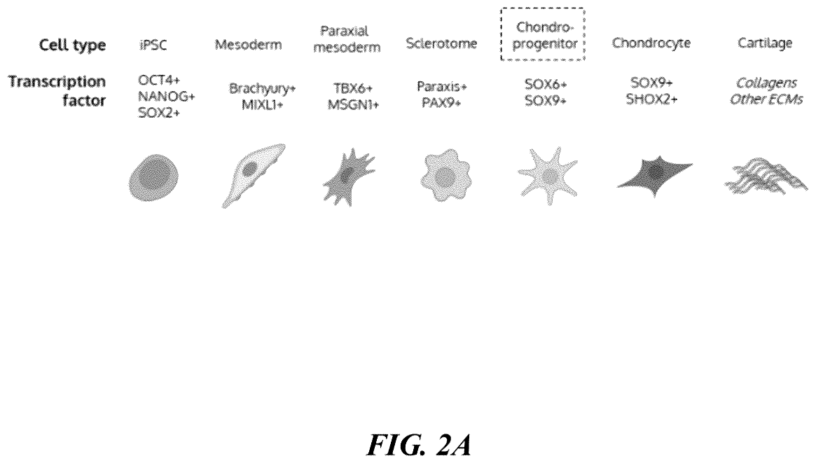

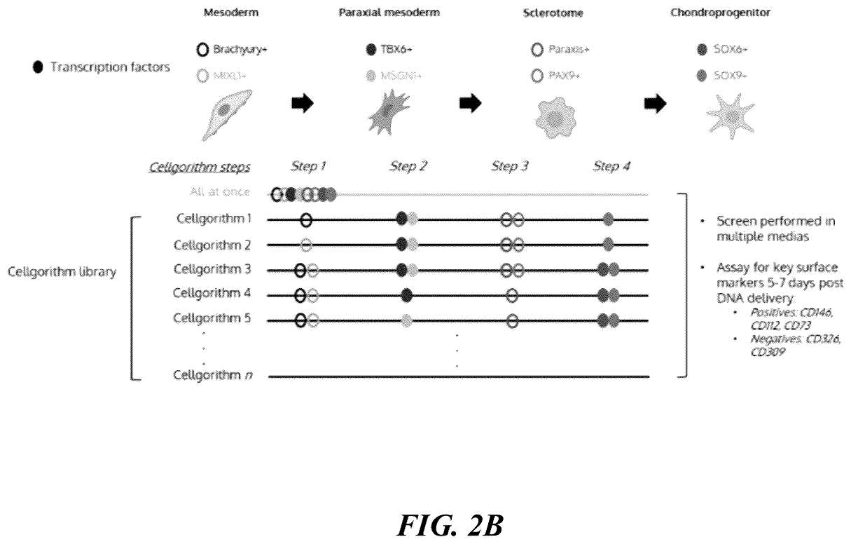

In this example, mesodermal stem cells (MSCs) were formed into chondroprogenitor cells using heterologous genetic circuits. Differentiation of a stem cell (e.g., MSC) to a chondroprogenitor cell can be a complex process requiring turning on a plurality of endogenous genes at different time points, while turning off a plurality of endogenous genes at different time points. See FIG. 2A for examples of different endogenous genes that are induced for expression at different stages of a differentiation of a stem cell to a chondroprogenitor cell, then to a chondrocyte. Thus, one or more heterologous genetic circuits as disclosed herein can be utilized to automatically promote regulation of such cascade of different endogenous gene expressions. In some cases, each heterologous genetic circuit can be configured to regulate expression levels of a plurality of genes at a plurality of different time points upon a single activation of such heterologous genetic circuit.

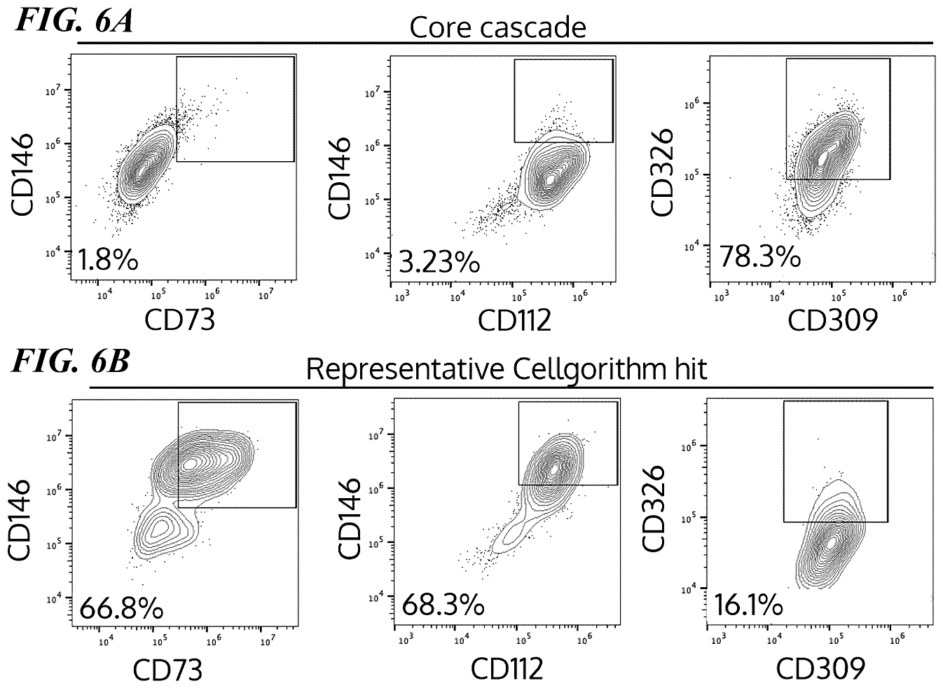

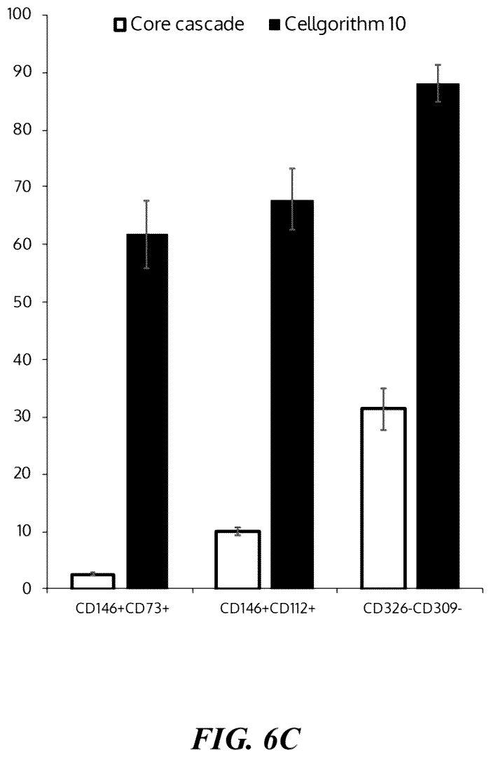

The MSCs were transiently transfected with plasmid DNAs encoding one of the heterologous genetic circuits as described in FIG. 2B or FIG. 3, e.g., targeting a combination of TBXT, MIXL1, TBX6, MSGN1, UNCX, TCF15, PAX9, SOX6, SOX9, PAX3, PAX7, and COL2A1. All genes targeted were activated. Flow cytometry was used to analyze for CD146/CD73 double positive cells, CD146/CD112 double positive cells, and CD326/CD309 double negative cells, each of which indicated the formation of chondrogenic progenitor cells (FIGS. 6A-6B). Top performing HCGs, such as Cellgorithm 10, were found to convert about 60% of cells into chondrogenic progenitor cells in four days (FIG. 6C).

### B. Characterization of Chondroprogenitor Cells Generated by a Heterologous Genetic Circuit.

Transient plasmid delivery of the heterologous genetic circuits induced double positive and double negative chondrogenic progenitor cell markers to appear after four days. As shown in FIG. 6C, flow analysis revealed that at least one of the heterologous genetic circuits, heterologous genetic circuit 10 (e.g., Cellgorithm 10), provided in FIG. 3 yielded at least about 60% MSC to chondroprogenitor cell conversion in four days. This conversion rate was greater than a control population of iPSCs that were treated by activating the same endogenous genes, but all at the same time.

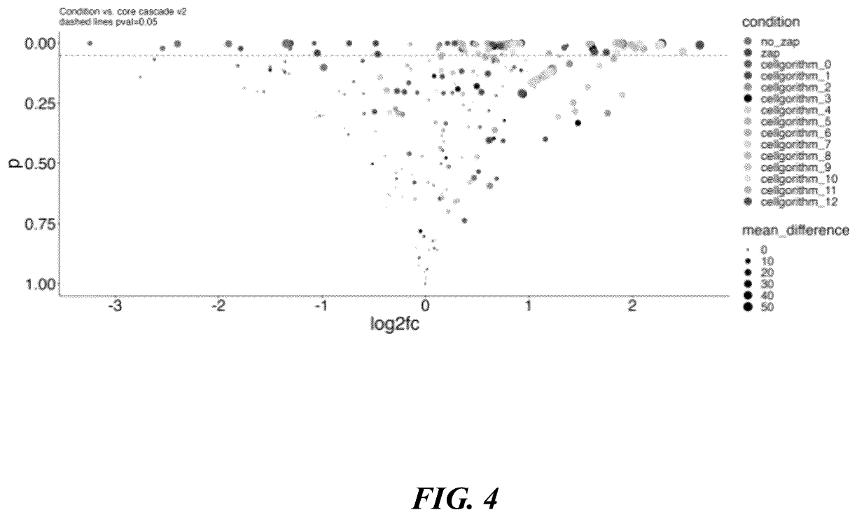

Separately, cells treated with various heterologous genetic circuits from FIG. 3 were analyzed (e.g., via flow) for the positive markers of chondroprogenitor cells (e.g., CD146+/CD73+, CD146+/CD73+, CD326−/CD309−, etc.), then plotted in a volcano plot, as shown in FIG. 4. The volcano plot was generated to compare the efficiency of each heterologous genetic circuit in terms of (i) statistical significance (p-value) as compared to the all-at-once control circuit that activated the target endogenous genes at once, versus (ii) magnitude of change (fold change) of the positive markers of chondroprogenitor cells as compared to the all-at-once control circuit.

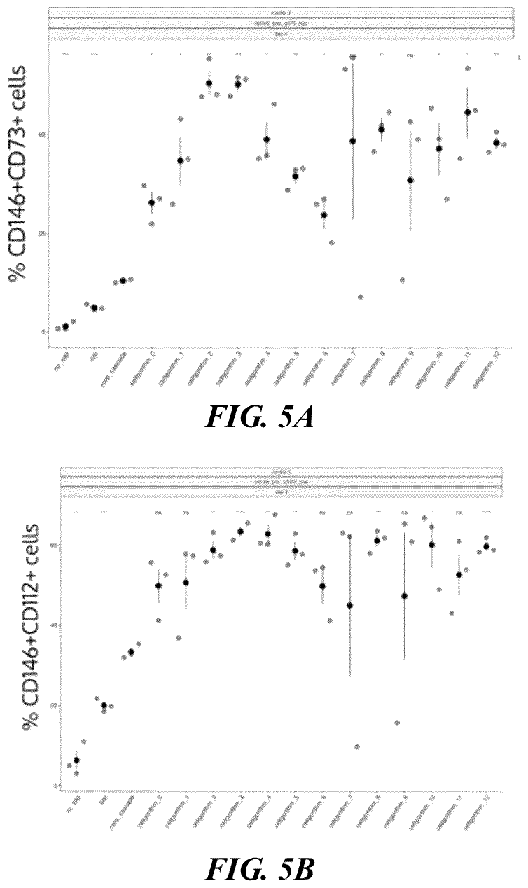

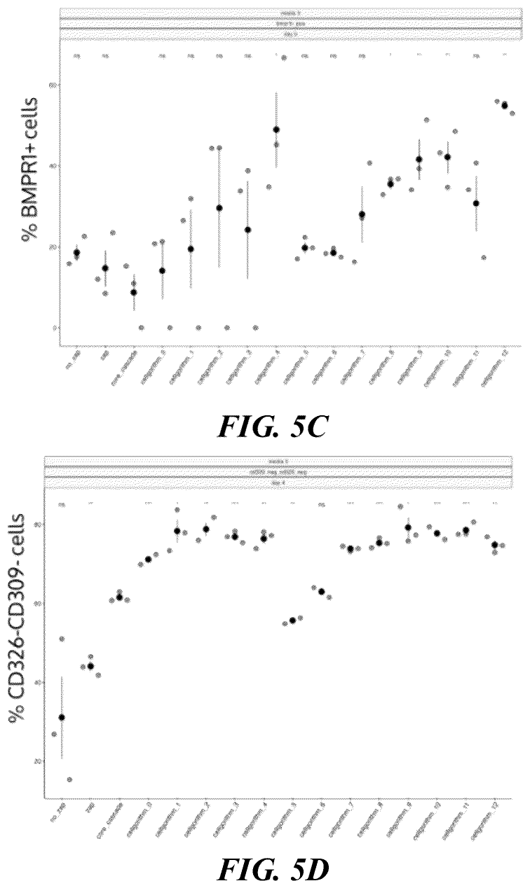

FIGS. 5A-5D show representative data utilized in the volcano plot of FIG. 4. In each plot of FIGS. 5A-5D, the y-axis represents proportion of each cell sample that express the indicated chondroprogenitor cell markers (e.g., CD146+/CD73+, CD146+/CD73+, BMPR1+, or CD326−/CD309−) upon treatment with one of the heterologous genetic circuits from FIG. 3. The x-axis indicates which heterologous genetic circuit was utilized for each cell sample. The plots in FIGS. 5A-5D show that various heterologous genetic circuits (e.g., Cellgorithm 9, Cellgorithm 10, Cellgorithm 12, etc.) can generate chondroprogenitor cells as indicated by multiple chondroprogenitor cell marker panels.

### Example 2: Chondrogenesis Assays

In this prophetic example, iPSCs and mesenchymal cells are contacted with top-performing HGCs from Example 1 and allowed to grow in media until chondrocytes form. Control chondrocyte populations are collected and purified from mouse samples. Chondrocytes from both the HGC and control populations are tested for chondrogenesis, including amount of cartilage formed and the mechanical properties of the cartilage tested.

### Example 3: Implantation and Engraftment of Chondrogenic Progenitor Cells

Tissue-specific cells (e.g., chondroprogenitor cells) as prepared by the systems and methods of the present disclosure can be administered to a subject in need thereof (e.g., injected into a joint tissue), to treat a joint-related disorder or a cartilage-related disorder of a subject.

### A. Generation of Chondroprogenitor Cells.

Stem cells (e.g., MPSCs) can be transduced or transfected (e.g., transiently transfected) with one or more heterologous genes (e.g., plasmid DNAs) encoding a at least one heterologous genetic circuit, such as, for example, one of the respective heterologous genetic circuit as shown in FIG. 3, in accordance with the methods described in Example 1, in order to generate chondroprogenitor cells.

### B. In Vivo Administration of Chondroprogenitor Cells.

Upon generation of chondroprogenitor cells by systems and methods of the present disclosure, the chondroprogenitor cells (e.g., CD146 cells) can be purified using anti-CD146 antibody. Cells can be concentrated and resuspended in a buffer (e.g., PBS), and then be administered to mice via injection directly into the joint or other site of interest. After 8 to 10 weeks, the mice can be sacrificed, and the tissue surrounding the area of injection can be sectioned. Sections can be immunostained for human cartilage to confirm the engraftment of the ex vivo-generated chondroprogenitor cells.

An additional protocol for chondroprogenitor cell transplantation is provided herein. Upon generation as described herein, the chondroprogenitor cells can be suspended in chondrogenic cell media. These cells can be transplanted into a target site in a joint with or without further expansion. For expansion, the chondroprogenitor cells can be plated into tissue culture wells containing hydrogels (flat or patterned) or thin gel coated plastic (flat or patterned) as sparse cultures (e.g., 1000-2000 cells/well of a 24 well size plate) and cultured while replacing media every 3 days. On the day of transplantation, NOD/SCID mice can be anesthetized by intraperitoneal injection of Ketamine (2.4 mg/mouse) and Xylazine (240 g/mouse) and hindlimb irradiated as previously described (A. Sacco et al. (2008) Nature 456, 502). The generated chondroprogenitor cells can be counted and resuspended, and subsequently injected into recipient mice.

Engraftment (e.g., differentiation and integration into the local chondrogenic tissue) of the transplanted chondroprogenitor cells can be visualized by various methods. For example, the chondroprogenitor cells can be engineered to express a heterologous marker (e.g., fluorescent proteins, such as green fluorescent protein) that is not present in the transplanted animal. Alternatively or in addition to, the chondroprogenitor cells can be allogeneic to the animal, such that any cartilage that are differentiated from the chondroprogenitor cells upon the transplantation can be identified (e.g., immunostaining) by an antigen that is not found in the transplanted animal.

### Example 4: Creating Chondrogenic Cells In Vitro

For an improved cell manufacturing, cells of interest can be engineered to exhibit increased expression of genes that will alter their phenotype into the chondrogenic cell lineage. In some embodiments, improved cell manufacturing can be evidenced by shorter time to differentiate stem cells (e.g., pluripotent stem cells) to target cells (e.g., chondrogenic precursor or chondrogenic cells), increased number of target cells as compared to other protocols, enhanced level of cartilage formation as evidenced by collagen, aggrecan, and/or ECM protein production, lower cost to produce a similar number of target cells, and more. Upon nucleofection of a Cellgorithm into the cells of interest these genes will be sequentially expressed within the cell of interest The cells of interest can be induced pluripotent stem cells. Cells post engineering can exhibit increased presence of markers of the chondrogenic cell lineage by measurements of RNA transcription, can exhibit DNA accessibility indicative of cells in the chondrogenic cell lineage, and/or increased function in cartilage formation assays measuring 1) collagen, aggrecan, and desired ECM protein production 2) cartilage pellet formation whereby the cartilage pellets express desired levels of collagen, aggrecan, and desired ECM proteins.

For example a library of Cellgorithms including but not limited to Cellgorithms labeled in Table 1 which are built from sequences with core function units described in SEQ ID NOs: 1-1932 can be introduced to cells of interest. Table 1 shows 1932 unique chondro constructs (e.g., There are 23 unique stem combinations. There are 21 unique “4-pools” of chondro spacers. There are 4 pools because for each gene all 4 constructs targeting that gene are used together. Therefore, there are 21*23=483 different combinations If each individual construct is counted, multiply by 4. There are 483*4=1932 unique chondro constructs).

### Example 5: In Vivo Validation of Cellgorithm-Derived Chondrogenic Cells

Chondrogenic cells generated using Cellgorithm technology can be used as a regenerative therapy for individuals with defective cartilage function, cartilage damage, or arthritic cartilage. The delivery of these cells into such individuals can provide an alleviation of pain in the associated joints, improved joint function, and improved longevity of the function in respective treated joints. Once produced by Cellgorithm-driven instructions, the chondrogenic cells can be used in the same manner as chondrogenic cells produced by other methods, such as isolation from cadaveric donors, or differentiation of pluripotent stem cells using protocols for changing growth factors and other small molecules over a period of time (so called directed differentiation). Regardless of their origin, chondrogenic cells can be evaluated and used in vivo by injecting them into the joints of animal models, whereby the joint bad undergone acute cartilage injury or it had arthritis via genetic model or induction. Chondrogenic cells can be phenotypically characterized based on gene expression and cell surface marker characteristics, and can be functionally characterized for their ability to create cartilage. Both in vitro and in vivo characterizations provide significant insight into the quality of the chondrogenic cells.

To evaluate function of the produced chondrogenic cells can be implanted into osteochondral defected knee joints (e.g., of full thickness, 1 mm in diameter) of athymic rats (e.g., n=6) or 6 month old Yucatan pigs. Following transplantation of chondrogenic cells, histology can be performed to measure genes of interest (e.g., COL2A1, COL2A2, COL10, AGGN) and extracellular matrix proteins of interest. The chondrogenic cells should engraft to create cartilage expressing these proteins beyond the vehicle control, providing healing of the defected cartilage that translates to functional joint outcomes.

TABLE 1
Concatenated proGuide Sequences

| Gene | | |
|---|---|---|
| target | seq | |
| (if app- | id | |
| licable) | no: | full sequence |
| ACAN | 1 | g-TGTGACACATACAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| ACAN | 2 | -GGGCGGCCGACAGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| ACAN | 3 | -GTGCCCGGACTCTG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| ACAN | 4 | g-CCCTGGCTCCGGAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| ACAN | 5 | g-TGTGACACATACAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| ACAN | 6 | -GGGCGGCCGACAGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| ACAN | 7 | -GTGCCCGGACTCTG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| ACAN | 8 | g-CCCTGGCTCCGGAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| ACAN | 9 | g-TGTGACACATACAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| ACAN | 10 | -GGGCGGCCGACAGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| ACAN | 11 | -GTGCCCGGACTCTG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| ACAN | 12 | g-CCCTGGCTCCGGAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| ACAN | 13 | g-TGTGACACATACAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| ACAN | 14 | -GGGCGGCCGACAGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| ACAN | 15 | -GTGCCCGGACTCTG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| ACAN | 16 | g-CCCTGGCTCCGGAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| ACAN | 17 | g-TGTGACACATACAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| ACAN | 18 | -GGGCGGCCGACAGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| ACAN | 19 | -GTGCCCGGACTCTG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| ACAN | 20 | g-CCCTGGCTCCGGAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| ACAN | 21 | g-TGTGACACATACAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| ACAN | 22 | -GGGCGGCCGACAGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| ACAN | 23 | -GTGCCCGGACTCTG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| ACAN | 24 | g-CCCTGGCTCCGGAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| ACAN | 25 | g-TGTGACACATACAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| ACAN | 26 | -GGGCGGCCGACAGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| ACAN | 27 | -GTGCCCGGACTCTG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| ACAN | 28 | g-CCCTGGCTCCGGAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| ACAN | 29 | g-TGTGACACATACAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| ACAN | 30 | -GGGCGGCCGACAGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| ACAN | 31 | -GTGCCCGGACTCTG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| ACAN | 32 | g-CCCTGGCTCCGGAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| ACAN | 33 | g-TGTGACACATACAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| ACAN | 34 | -GGGCGGCCGACAGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| ACAN | 35 | -GTGCCCGGACTCTG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| ACAN | 36 | g-CCCTGGCTCCGGAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| ACAN | 37 | g-TGTGACACATACAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| ACAN | 38 | -GGGCGGCCGACAGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| ACAN | 39 | -GTGCCCGGACTCTG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| ACAN | 40 | g-CCCTGGCTCCGGAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| ACAN | 41 | g-TGTGACACATACAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| ACAN | 42 | -GGGCGGCCGACAGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| ACAN | 43 | -GTGCCCGGACTCTG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| ACAN | 44 | g-CCCTGGCTCCGGAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| ACAN | 45 | g-TGTGACACATACAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| ACAN | 46 | -GGGCGGCCGACAGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| ACAN | 47 | -GTGCCCGGACTCTG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| ACAN | 48 | g-CCCTGGCTCCGGAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| ACAN | 49 | g-TGTGACACATACAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| ACAN | 50 | -GGGCGGCCGACAGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| ACAN | 51 | -GTGCCCGGACTCTG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| ACAN | 52 | g-CCCTGGCTCCGGAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| ACAN | 53 | g-TGTGACACATACAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| ACAN | 54 | -GGGCGGCCGACAGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| ACAN | 55 | -GTGCCCGGACTCTG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| ACAN | 56 | g-CCCTGGCTCCGGAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| ACAN | 57 | g-TGTGACACATACAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| ACAN | 58 | -GGGCGGCCGACAGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| ACAN | 59 | -GTGCCCGGACTCTG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| ACAN | 60 | g-CCCTGGCTCCGGAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| ACAN | 61 | g-TGTGACACATACAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| ACAN | 62 | -GGGCGGCCGACAGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| ACAN | 63 | -GTGCCCGGACTCTG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| ACAN | 64 | g-CCCTGGCTCCGGAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| ACAN | 65 | g-TGTGACACATACAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| ACAN | 66 | -GGGCGGCCGACAGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| ACAN | 67 | -GTGCCCGGACTCTG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| ACAN | 68 | g-CCCTGGCTCCGGAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| ACAN | 69 | g-TGTGACACATACAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| ACAN | 70 | -GGGCGGCCGACAGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| ACAN | 71 | -GTGCCCGGACTCTG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| ACAN | 72 | g-CCCTGGCTCCGGAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| ACAN | 73 | g-TGTGACACATACAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| ACAN | 74 | -GGGCGGCCGACAGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| ACAN | 75 | -GTGCCCGGACTCTG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| ACAN | 76 | g-CCCTGGCTCCGGAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| ACAN | 77 | g-TGTGACACATACAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| ACAN | 78 | -GGGCGGCCGACAGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| ACAN | 79 | -GTGCCCGGACTCTG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| ACAN | 80 | g-CCCTGGCTCCGGAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| ACAN | 81 | g-TGTGACACATACAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| ACAN | 82 | -GGGCGGCCGACAGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| ACAN | 83 | -GTGCCCGGACTCTG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| ACAN | 84 | g-CCCTGGCTCCGGAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| ACAN | 85 | g-TGTGACACATACAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| ACAN | 86 | -GGGCGGCCGACAGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| ACAN | 87 | -GTGCCCGGACTCTG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| ACAN | 88 | g-CCCTGGCTCCGGAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| ACAN | 89 | g-TGTGACACATACAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| ACAN | 90 | -GGGCGGCCGACAGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| ACAN | 91 | -GTGCCCGGACTCTG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| ACAN | 92 | g-CCCTGGCTCCGGAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| COL2A1 | 93 | g-CGCCAGCCTCGAAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| COL2A1 | 94 | -GAACCGCCCGCCCC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| COL2A1 | 95 | g-CGGCGCACTAGGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| COL2A1 | 96 | -GCCCCGGGTTTGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| COL2A1 | 97 | g-CGCCAGCCTCGAAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| COL2A1 | 98 | -GAACCGCCCGCCCC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| COL2A1 | 99 | g-CGGCGCACTAGGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| COL2A1 | 100 | -GCCCCGGGTTTGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| COL2A1 | 101 | g-CGCCAGCCTCGAAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| COL2A1 | 102 | -GAACCGCCCGCCCC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| COL2A1 | 103 | g-CGGCGCACTAGGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| COL2A1 | 104 | -GCCCCGGGTTTGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| COL2A1 | 105 | g-CGCCAGCCTCGAAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| COL2A1 | 106 | -GAACCGCCCGCCCC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| COL2A1 | 107 | g-CGGCGCACTAGGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| COL2A1 | 108 | -GCCCCGGGTTTGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| COL2A1 | 109 | g-CGCCAGCCTCGAAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| COL2A1 | 110 | -GAACCGCCCGCCCC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| COL2A1 | 111 | g-CGGCGCACTAGGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| COL2A1 | 112 | -GCCCCGGGTTTGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| COL2A1 | 113 | g-CGCCAGCCTCGAAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| COL2A1 | 114 | -GAACCGCCCGCCCC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| COL2A1 | 115 | g-CGGCGCACTAGGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| COL2A1 | 116 | -GCCCCGGGTTTGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| COL2A1 | 117 | g-CGCCAGCCTCGAAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| COL2A1 | 118 | -GAACCGCCCGCCCC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| COL2A1 | 119 | g-CGGCGCACTAGGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| COL2A1 | 120 | -GCCCCGGGTTTGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| COL2A1 | 121 | g-CGCCAGCCTCGAAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| COL2A1 | 122 | -GAACCGCCCGCCCC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| COL2A1 | 123 | g-CGGCGCACTAGGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| COL2A1 | 124 | -GCCCCGGGTTTGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| COL2A1 | 125 | g-CGCCAGCCTCGAAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| COL2A1 | 126 | -GAACCGCCCGCCCC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| COL2A1 | 127 | g-CGGCGCACTAGGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| COL2A1 | 128 | -GCCCCGGGTTTGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| COL2A1 | 129 | g-CGCCAGCCTCGAAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| COL2A1 | 130 | -GAACCGCCCGCCCC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| COL2A1 | 131 | g-CGGCGCACTAGGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| COL2A1 | 132 | -GCCCCGGGTTTGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| COL2A1 | 133 | g-CGCCAGCCTCGAAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| COL2A1 | 134 | -GAACCGCCCGCCCC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| COL2A1 | 135 | g-CGGCGCACTAGGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| COL2A1 | 136 | -GCCCCGGGTTTGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| COL2A1 | 137 | g-CGCCAGCCTCGAAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| COL2A1 | 138 | -GAACCGCCCGCCCC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| COL2A1 | 139 | g-CGGCGCACTAGGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| COL2A1 | 140 | -GCCCCGGGTTTGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| COL2A1 | 141 | g-CGCCAGCCTCGAAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| COL2A1 | 142 | -GAACCGCCCGCCCC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| COL2A1 | 143 | g-CGGCGCACTAGGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| COL2A1 | 144 | -GCCCCGGGTTTGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| COL2A1 | 145 | g-CGCCAGCCTCGAAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| COL2A1 | 146 | -GAACCGCCCGCCCC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| COL2A1 | 147 | g-CGGCGCACTAGGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| COL2A1 | 148 | -GCCCCGGGTTTGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| COL2A1 | 149 | g-CGCCAGCCTCGAAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| COL2A1 | 150 | -GAACCGCCCGCCCC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| COL2A1 | 151 | g-CGGCGCACTAGGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| COL2A1 | 152 | -GCCCCGGGTTTGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| COL2A1 | 153 | g-CGCCAGCCTCGAAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| COL2A1 | 154 | -GAACCGCCCGCCCC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| COL2A1 | 155 | g-CGGCGCACTAGGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| COL2A1 | 156 | -GCCCCGGGTTTGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| COL2A1 | 157 | g-CGCCAGCCTCGAAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| COL2A1 | 158 | -GAACCGCCCGCCCC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| COL2A1 | 159 | g-CGGCGCACTAGGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| COL2A1 | 160 | -GCCCCGGGTTTGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| COL2A1 | 161 | g-CGCCAGCCTCGAAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| COL2A1 | 162 | -GAACCGCCCGCCCC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| COL2A1 | 163 | g-CGGCGCACTAGGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| COL2A1 | 164 | -GCCCCGGGTTTGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| COL2A1 | 165 | g-CGCCAGCCTCGAAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| COL2A1 | 166 | -GAACCGCCCGCCCC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| COL2A1 | 167 | g-CGGCGCACTAGGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| COL2A1 | 168 | -GCCCCGGGTTTGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| COL2A1 | 169 | g-CGCCAGCCTCGAAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| COL2A1 | 170 | -GAACCGCCCGCCCC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| COL2A1 | 171 | g-CGGCGCACTAGGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| COL2A1 | 172 | -GCCCCGGGTTTGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| COL2A1 | 173 | g-CGCCAGCCTCGAAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| COL2A1 | 174 | -GAACCGCCCGCCCC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| COL2A1 | 175 | g-CGGCGCACTAGGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| COL2A1 | 176 | -GCCCCGGGTTTGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| COL2A1 | 177 | g-CGCCAGCCTCGAAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| COL2A1 | 178 | -GAACCGCCCGCCCC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| COL2A1 | 179 | g-CGGCGCACTAGGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| COL2A1 | 180 | -GCCCCGGGTTTGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| COL2A1 | 181 | g-CGCCAGCCTCGAAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| COL2A1 | 182 | -GAACCGCCCGCCCC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| COL2A1 | 183 | g-CGGCGCACTAGGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| COL2A1 | 184 | -GCCCCGGGTTTGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| MEF2D | 185 | g-CCGCAGCCTAGCTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| MEF2D | 186 | g-TGACGGACAGGCGT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| MEF2D | 187 | g-CGGGGCCGCGTCCT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| MEF2D | 188 | g-CAGAGGCCTTTAAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| MEF2D | 189 | g-CCGCAGCCTAGCTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| MEF2D | 190 | g-TGACGGACAGGCGT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| MEF2D | 191 | g-CGGGGCCGCGTCCT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| MEF2D | 192 | g-CAGAGGCCTTTAAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| MEF2D | 193 | g-CCGCAGCCTAGCTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| MEF2D | 194 | g-TGACGGACAGGCGT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| MEF2D | 195 | g-CGGGGCCGCGTCCT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| MEF2D | 196 | g-CAGAGGCCTTTAAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| MEF2D | 197 | g-CCGCAGCCTAGCTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| MEF2D | 198 | g-TGACGGACAGGCGT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| MEF2D | 199 | g-CGGGGCCGCGTCCT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| MEF2D | 200 | g-CAGAGGCCTTTAAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| MEF2D | 201 | g-CCGCAGCCTAGCTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| MEF2D | 202 | g-TGACGGACAGGCGT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| MEF2D | 203 | g-CGGGGCCGCGTCCT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| MEF2D | 204 | g-CAGAGGCCTTTAAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| MEF2D | 205 | g-CCGCAGCCTAGCTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| MEF2D | 206 | g-TGACGGACAGGCGT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| MEF2D | 207 | g-CGGGGCCGCGTCCT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| MEF2D | 208 | g-CAGAGGCCTTTAAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| MEF2D | 209 | g-CCGCAGCCTAGCTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| MEF2D | 210 | g-TGACGGACAGGCGT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| MEF2D | 211 | g-CGGGGCCGCGTCCT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| MEF2D | 212 | g-CAGAGGCCTTTAAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| MEF2D | 213 | g-CCGCAGCCTAGCTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| MEF2D | 214 | g-TGACGGACAGGCGT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| MEF2D | 215 | g-CGGGGCCGCGTCCT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| MEF2D | 216 | g-CAGAGGCCTTTAAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| MEF2D | 217 | g-CCGCAGCCTAGCTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| MEF2D | 218 | g-TGACGGACAGGCGT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| MEF2D | 219 | g-CGGGGCCGCGTCCT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| MEF2D | 220 | g-CAGAGGCCTTTAAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| MEF2D | 221 | g-CCGCAGCCTAGCTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| MEF2D | 222 | g-TGACGGACAGGCGT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| MEF2D | 223 | g-CGGGGCCGCGTCCT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| MEF2D | 224 | g-CAGAGGCCTTTAAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| MEF2D | 225 | g-CCGCAGCCTAGCTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| MEF2D | 226 | g-TGACGGACAGGCGT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| MEF2D | 227 | g-CGGGGCCGCGTCCT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| MEF2D | 228 | g-CAGAGGCCTTTAAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| MEF2D | 229 | g-CCGCAGCCTAGCTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| MEF2D | 230 | g-TGACGGACAGGCGT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| MEF2D | 231 | g-CGGGGCCGCGTCCT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| MEF2D | 232 | g-CAGAGGCCTTTAAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| MEF2D | 233 | g-CCGCAGCCTAGCTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| MEF2D | 234 | g-TGACGGACAGGCGT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| MEF2D | 235 | g-CGGGGCCGCGTCCT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| MEF2D | 236 | g-CAGAGGCCTTTAAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| MEF2D | 237 | g-CCGCAGCCTAGCTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| MEF2D | 238 | g-TGACGGACAGGCGT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| MEF2D | 239 | g-CGGGGCCGCGTCCT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| MEF2D | 240 | g-CAGAGGCCTTTAAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| MEF2D | 241 | g-CCGCAGCCTAGCTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| MEF2D | 242 | g-TGACGGACAGGCGT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| MEF2D | 243 | g-CGGGGCCGCGTCCT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| MEF2D | 244 | g-CAGAGGCCTTTAAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| MEF2D | 245 | g-CCGCAGCCTAGCTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| MEF2D | 246 | g-TGACGGACAGGCGT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| MEF2D | 247 | g-CGGGGCCGCGTCCT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| MEF2D | 248 | g-CAGAGGCCTTTAAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| MEF2D | 249 | g-CCGCAGCCTAGCTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| MEF2D | 250 | g-TGACGGACAGGCGT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| MEF2D | 251 | g-CGGGGCCGCGTCCT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| MEF2D | 252 | g-CAGAGGCCTTTAAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| MEF2D | 253 | g-CCGCAGCCTAGCTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| MEF2D | 254 | g-TGACGGACAGGCGT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| MEF2D | 255 | g-CGGGGCCGCGTCCT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| MEF2D | 256 | g-CAGAGGCCTTTAAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| MEF2D | 257 | g-CCGCAGCCTAGCTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| MEF2D | 258 | g-TGACGGACAGGCGT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| MEF2D | 259 | g-CGGGGCCGCGTCCT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| MEF2D | 260 | g-CAGAGGCCTTTAAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| MEF2D | 261 | g-CCGCAGCCTAGCTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| MEF2D | 262 | g-TGACGGACAGGCGT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| MEF2D | 263 | g-CGGGGCCGCGTCCT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| MEF2D | 264 | g-CAGAGGCCTTTAAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| MEF2D | 265 | g-CCGCAGCCTAGCTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| MEF2D | 266 | g-TGACGGACAGGCGT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| MEF2D | 267 | g-CGGGGCCGCGTCCT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| MEF2D | 268 | g-CAGAGGCCTTTAAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| MEF2D | 269 | g-CCGCAGCCTAGCTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| MEF2D | 270 | g-TGACGGACAGGCGT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| MEF2D | 271 | g-CGGGGCCGCGTCCT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| MEF2D | 272 | g-CAGAGGCCTTTAAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| MEF2D | 273 | g-CCGCAGCCTAGCTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| MEF2D | 274 | g-TGACGGACAGGCGT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| MEF2D | 275 | g-CGGGGCCGCGTCCT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| MEF2D | 276 | g-CAGAGGCCTTTAAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 277 | -GCGTGGGGTAAGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 278 | g-CACGGCAATTTCTC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 279 | -GTGACGCTAATATG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 280 | g-CCTAGGGGTAGGGA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 281 | g-AGGGGCGGGTCCGA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 282 | g-CTAAGCAGCAACGT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 283 | -GCTAGCTCCCCACC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 284 | g-AGCTGCATGGTGAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| pax9 | 285 | -GCGTGGGGTAAGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 286 | g-CACGGCAATTTCTC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 287 | -GTGACGCTAATATG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 288 | g-CCTAGGGGTAGGGA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 289 | g-AGGGGCGGGTCCGA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 290 | g-CTAAGCAGCAACGT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 291 | -GCTAGCTCCCCACC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 292 | g-AGCTGCATGGTGAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| pax9 | 293 | -GCGTGGGGTAAGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 294 | g-CACGGCAATTTCTC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 295 | -GTGACGCTAATATG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 296 | g-CCTAGGGGTAGGGA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 297 | g-AGGGGCGGGTCCGA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 298 | g-CTAAGCAGCAACGT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 299 | -GCTAGCTCCCCACC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 300 | g-AGCTGCATGGTGAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| pax9 | 301 | -GCGTGGGGTAAGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 302 | g-CACGGCAATTTCTC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 303 | -GTGACGCTAATATG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 304 | g-CCTAGGGGTAGGGA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 305 | g-AGGGGCGGGTCCGA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 306 | g-CTAAGCAGCAACGT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 307 | -GCTAGCTCCCCACC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 308 | g-AGCTGCATGGTGAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| pax9 | 309 | -GCGTGGGGTAAGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 310 | g-CACGGCAATTTCTC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 311 | -GTGACGCTAATATG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 312 | g-CCTAGGGGTAGGGA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 313 | g-AGGGGCGGGTCCGA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 314 | g-CTAAGCAGCAACGT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 315 | -GCTAGCTCCCCACC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 316 | g-AGCTGCATGGTGAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| pax9 | 317 | -GCGTGGGGTAAGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 318 | g-CACGGCAATTTCTC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 319 | -GTGACGCTAATATG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 320 | g-CCTAGGGGTAGGGA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 321 | g-AGGGGCGGGTCCGA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 322 | g-CTAAGCAGCAACGT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 323 | -GCTAGCTCCCCACC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 324 | g-AGCTGCATGGTGAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| pax9 | 325 | -GCGTGGGGTAAGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 326 | g-CACGGCAATTTCTC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 327 | -GTGACGCTAATATG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 328 | g-CCTAGGGGTAGGGA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 329 | g-AGGGGCGGGTCCGA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 330 | g-CTAAGCAGCAACGT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 331 | -GCTAGCTCCCCACC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 332 | g-AGCTGCATGGTGAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| pax9 | 333 | -GCGTGGGGTAAGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 334 | g-CACGGCAATTTCTC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 335 | -GTGACGCTAATATG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 336 | g-CCTAGGGGTAGGGA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 337 | g-AGGGGCGGGTCCGA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 338 | g-CTAAGCAGCAACGT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 339 | -GCTAGCTCCCCACC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 340 | g-AGCTGCATGGTGAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| pax9 | 341 | -GCGTGGGGTAAGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 342 | g-CACGGCAATTTCTC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 343 | -GTGACGCTAATATG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 344 | g-CCTAGGGGTAGGGA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 345 | g-AGGGGCGGGTCCGA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 346 | g-CTAAGCAGCAACGT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 347 | -GCTAGCTCCCCACC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 348 | g-AGCTGCATGGTGAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| pax9 | 349 | -GCGTGGGGTAAGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 350 | g-CACGGCAATTTCTC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 351 | -GTGACGCTAATATG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 352 | g-CCTAGGGGTAGGGA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 353 | g-AGGGGCGGGTCCGA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 354 | g-CTAAGCAGCAACGT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 355 | -GCTAGCTCCCCACC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 356 | g-AGCTGCATGGTGAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| pax9 | 357 | -GCGTGGGGTAAGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 358 | g-CACGGCAATTTCTC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 359 | -GTGACGCTAATATG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 360 | g-CCTAGGGGTAGGGA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 361 | g-AGGGGCGGGTCCGA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 362 | g-CTAAGCAGCAACGT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 363 | -GCTAGCTCCCCACC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 364 | g-AGCTGCATGGTGAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| pax9 | 365 | -GCGTGGGGTAAGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 366 | g-CACGGCAATTTCTC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 367 | -GTGACGCTAATATG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 368 | g-CCTAGGGGTAGGGA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 369 | g-AGGGGCGGGTCCGA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 370 | g-CTAAGCAGCAACGT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 371 | -GCTAGCTCCCCACC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 372 | g-AGCTGCATGGTGAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| pax9 | 373 | -GCGTGGGGTAAGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 374 | g-CACGGCAATTTCTC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 375 | -GTGACGCTAATATG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 376 | g-CCTAGGGGTAGGGA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 377 | g-AGGGGCGGGTCCGA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 378 | g-CTAAGCAGCAACGT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 379 | -GCTAGCTCCCCACC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 380 | g-AGCTGCATGGTGAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| pax9 | 381 | -GCGTGGGGTAAGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 382 | g-CACGGCAATTTCTC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 383 | -GTGACGCTAATATG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 384 | g-CCTAGGGGTAGGGA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 385 | g-AGGGGCGGGTCCGA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 386 | g-CTAAGCAGCAACGT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 387 | -GCTAGCTCCCCACC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 388 | g-AGCTGCATGGTGAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| pax9 | 389 | -GCGTGGGGTAAGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 390 | g-CACGGCAATTTCTC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 391 | -GTGACGCTAATATG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 392 | g-CCTAGGGGTAGGGA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 393 | g-AGGGGCGGGTCCGA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 394 | g-CTAAGCAGCAACGT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 395 | -GCTAGCTCCCCACC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 396 | g-AGCTGCATGGTGAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| pax9 | 397 | -GCGTGGGGTAAGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 398 | g-CACGGCAATTTCTC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 399 | -GTGACGCTAATATG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 400 | g-CCTAGGGGTAGGGA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 401 | g-AGGGGCGGGTCCGA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 402 | g-CTAAGCAGCAACGT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 403 | -GCTAGCTCCCCACC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 404 | g-AGCTGCATGGTGAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| pax9 | 405 | -GCGTGGGGTAAGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 406 | g-CACGGCAATTTCTC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 407 | -GTGACGCTAATATG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 408 | g-CCTAGGGGTAGGGA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 409 | g-AGGGGCGGGTCCGA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 410 | g-CTAAGCAGCAACGT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 411 | -GCTAGCTCCCCACC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 412 | g-AGCTGCATGGTGAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| pax9 | 413 | -GCGTGGGGTAAGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 414 | g-CACGGCAATTTCTC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 415 | -GTGACGCTAATATG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 416 | g-CCTAGGGGTAGGGA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 417 | g-AGGGGCGGGTCCGA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 418 | g-CTAAGCAGCAACGT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 419 | -GCTAGCTCCCCACC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 420 | g-AGCTGCATGGTGAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| pax9 | 421 | -GCGTGGGGTAAGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 422 | g-CACGGCAATTTCTC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 423 | -GTGACGCTAATATG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 424 | g-CCTAGGGGTAGGGA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 425 | g-AGGGGCGGGTCCGA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 426 | g-CTAAGCAGCAACGT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 427 | -GCTAGCTCCCCACC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 428 | g-AGCTGCATGGTGAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| pax9 | 429 | -GCGTGGGGTAAGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 430 | g-CACGGCAATTTCTC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 431 | -GTGACGCTAATATG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 432 | g-CCTAGGGGTAGGGA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 433 | g-AGGGGCGGGTCCGA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 434 | g-CTAAGCAGCAACGT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 435 | -GCTAGCTCCCCACC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 436 | g-AGCTGCATGGTGAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| pax9 | 437 | -GCGTGGGGTAAGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 438 | g-CACGGCAATTTCTC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 439 | -GTGACGCTAATATG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 440 | g-CCTAGGGGTAGGGA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 441 | g-AGGGGCGGGTCCGA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 442 | g-CTAAGCAGCAACGT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 443 | -GCTAGCTCCCCACC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 444 | g-AGCTGCATGGTGAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| pax9 | 445 | -GCGTGGGGTAAGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 446 | g-CACGGCAATTTCTC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 447 | -GTGACGCTAATATG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 448 | g-CCTAGGGGTAGGGA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 449 | g-AGGGGCGGGTCCGA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 450 | g-CTAAGCAGCAACGT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 451 | -GCTAGCTCCCCACC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 452 | g-AGCTGCATGGTGAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| pax9 | 453 | -GCGTGGGGTAAGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 454 | g-CACGGCAATTTCTC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 455 | -GTGACGCTAATATG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 456 | g-CCTAGGGGTAGGGA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 457 | g-AGGGGCGGGTCCGA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 458 | g-CTAAGCAGCAACGT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 459 | -GCTAGCTCCCCACC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| pax9 | 460 | g-AGCTGCATGGTGAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 461 | g-CCTTACAGGAGTTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 462 | g-CACTATTACTGGAG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 463 | g-ACTGCCTACCACTG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 464 | g-TGTATCACATTCTG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 465 | g-AAGAGGTAAGTCGT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 466 | -gcgcgcggcaatgc-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 467 | g-AAGGAGCCTAGCCG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 468 | g-ACGCGGTGCCCCAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| RUNX2 | 469 | g-CCTTACAGGAGTTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 470 | g-CACTATTACTGGAG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 471 | g-ACTGCCTACCACTG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 472 | g-TGTATCACATTCTG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 473 | g-AAGAGGTAAGTCGT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 474 | -gcgcgcggcaatgc-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 475 | g-AAGGAGCCTAGCCG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 476 | g-ACGCGGTGCCCCAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| RUNX2 | 477 | g-CCTTACAGGAGTTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 478 | g-CACTATTACTGGAG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 479 | g-ACTGCCTACCACTG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 480 | g-TGTATCACATTCTG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 481 | g-AAGAGGTAAGTCGT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 482 | -gcgcgcggcaatgc-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 483 | g-AAGGAGCCTAGCCG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 484 | g-ACGCGGTGCCCCAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| RUNX2 | 485 | g-CCTTACAGGAGTTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 486 | g-CACTATTACTGGAG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 487 | g-ACTGCCTACCACTG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 488 | g-TGTATCACATTCTG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 489 | g-AAGAGGTAAGTCGT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 490 | -gcgcgcggcaatgc-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 491 | g-AAGGAGCCTAGCCG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 492 | g-ACGCGGTGCCCCAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| RUNX2 | 493 | g-CCTTACAGGAGTTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 494 | g-CACTATTACTGGAG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 495 | g-ACTGCCTACCACTG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 496 | g-TGTATCACATTCTG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 497 | g-AAGAGGTAAGTCGT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 498 | -gcgcgcggcaatgc-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 499 | g-AAGGAGCCTAGCCG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 500 | g-ACGCGGTGCCCCAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| RUNX2 | 501 | g-CCTTACAGGAGTTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 502 | g-CACTATTACTGGAG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 503 | g-ACTGCCTACCACTG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 504 | g-TGTATCACATTCTG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 505 | g-AAGAGGTAAGTCGT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 506 | -gcgcgcggcaatgc-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 507 | g-AAGGAGCCTAGCCG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 508 | g-ACGCGGTGCCCCAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| RUNX2 | 509 | g-CCTTACAGGAGTTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 510 | g-CACTATTACTGGAG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 511 | g-ACTGCCTACCACTG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 512 | g-TGTATCACATTCTG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 513 | g-AAGAGGTAAGTCGT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 514 | -gcgcgcggcaatgc-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 515 | g-AAGGAGCCTAGCCG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 516 | g-ACGCGGTGCCCCAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| RUNX2 | 517 | g-CCTTACAGGAGTTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 518 | g-CACTATTACTGGAG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 519 | g-ACTGCCTACCACTG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 520 | g-TGTATCACATTCTG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 521 | g-AAGAGGTAAGTCGT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 522 | -gcgcgcggcaatgc-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 523 | g-AAGGAGCCTAGCCG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 524 | g-ACGCGGTGCCCCAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| RUNX2 | 525 | g-CCTTACAGGAGTTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 526 | g-CACTATTACTGGAG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 527 | g-ACTGCCTACCACTG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 528 | g-TGTATCACATTCTG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 529 | g-AAGAGGTAAGTCGT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 530 | -gcgcgcggcaatgc-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 531 | g-AAGGAGCCTAGCCG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 532 | g-ACGCGGTGCCCCAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| RUNX2 | 533 | g-CCTTACAGGAGTTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 534 | g-CACTATTACTGGAG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 535 | g-ACTGCCTACCACTG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 536 | g-TGTATCACATTCTG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 537 | g-AAGAGGTAAGTCGT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 538 | -gcgcgcggcaatgc-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 539 | g-AAGGAGCCTAGCCG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 540 | g-ACGCGGTGCCCCAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| RUNX2 | 541 | g-CCTTACAGGAGTTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 542 | g-CACTATTACTGGAG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 543 | g-ACTGCCTACCACTG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 544 | g-TGTATCACATTCTG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 545 | g-AAGAGGTAAGTCGT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 546 | -gcgcgcggcaatgc-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 547 | g-AAGGAGCCTAGCCG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 548 | g-ACGCGGTGCCCCAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| RUNX2 | 549 | g-CCTTACAGGAGTTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 550 | g-CACTATTACTGGAG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 551 | g-ACTGCCTACCACTG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 552 | g-TGTATCACATTCTG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 553 | g-AAGAGGTAAGTCGT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 554 | -gcgcgcggcaatgc-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 555 | g-AAGGAGCCTAGCCG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 556 | g-ACGCGGTGCCCCAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| RUNX2 | 557 | g-CCTTACAGGAGTTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 558 | g-CACTATTACTGGAG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 559 | g-ACTGCCTACCACTG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 560 | g-TGTATCACATTCTG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 561 | g-AAGAGGTAAGTCGT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 562 | -gcgcgcggcaatgc-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 563 | g-AAGGAGCCTAGCCG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 564 | g-ACGCGGTGCCCCAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| RUNX2 | 565 | g-CCTTACAGGAGTTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 566 | g-CACTATTACTGGAG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 567 | g-ACTGCCTACCACTG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 568 | g-TGTATCACATTCTG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 569 | g-AAGAGGTAAGTCGT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 570 | -gcgcgcggcaatgc-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 571 | g-AAGGAGCCTAGCCG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 572 | g-ACGCGGTGCCCCAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| RUNX2 | 573 | g-CCTTACAGGAGTTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 574 | g-CACTATTACTGGAG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 575 | g-ACTGCCTACCACTG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 576 | g-TGTATCACATTCTG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 577 | g-AAGAGGTAAGTCGT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 578 | -gcgcgcggcaatgc-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 579 | g-AAGGAGCCTAGCCG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 580 | g-ACGCGGTGCCCCAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| RUNX2 | 581 | g-CCTTACAGGAGTTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 582 | g-CACTATTACTGGAG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 583 | g-ACTGCCTACCACTG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 584 | g-TGTATCACATTCTG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 585 | g-AAGAGGTAAGTCGT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 586 | -gcgcgcggcaatgc-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 587 | g-AAGGAGCCTAGCCG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 588 | g-ACGCGGTGCCCCAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| RUNX2 | 589 | g-CCTTACAGGAGTTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 590 | g-CACTATTACTGGAG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 591 | g-ACTGCCTACCACTG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 592 | g-TGTATCACATTCTG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 593 | g-AAGAGGTAAGTCGT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 594 | -gcgcgcggcaatgc-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 595 | g-AAGGAGCCTAGCCG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 596 | g-ACGCGGTGCCCCAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| RUNX2 | 597 | g-CCTTACAGGAGTTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 598 | g-CACTATTACTGGAG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 599 | g-ACTGCCTACCACTG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 600 | g-TGTATCACATTCTG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 601 | g-AAGAGGTAAGTCGT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 602 | -gcgcgcggcaatgc-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 603 | g-AAGGAGCCTAGCCG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 604 | g-ACGCGGTGCCCCAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| RUNX2 | 605 | g-CCTTACAGGAGTTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 606 | g-CACTATTACTGGAG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 607 | g-ACTGCCTACCACTG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 608 | g-TGTATCACATTCTG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 609 | g-AAGAGGTAAGTCGT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 610 | -gcgcgcggcaatgc-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 611 | g-AAGGAGCCTAGCCG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 612 | g-ACGCGGTGCCCCAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| RUNX2 | 613 | g-CCTTACAGGAGTTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 614 | g-CACTATTACTGGAG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 615 | g-ACTGCCTACCACTG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 616 | g-TGTATCACATTCTG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 617 | g-AAGAGGTAAGTCGT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 618 | -gcgcgcggcaatgc-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 619 | g-AAGGAGCCTAGCCG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 620 | g-ACGCGGTGCCCCAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| RUNX2 | 621 | g-CCTTACAGGAGTTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 622 | g-CACTATTACTGGAG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 623 | g-ACTGCCTACCACTG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 624 | g-TGTATCACATTCTG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 625 | g-AAGAGGTAAGTCGT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 626 | -gcgcgcggcaatgc-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 627 | g-AAGGAGCCTAGCCG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 628 | g-ACGCGGTGCCCCAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| RUNX2 | 629 | g-CCTTACAGGAGTTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 630 | g-CACTATTACTGGAG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 631 | g-ACTGCCTACCACTG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 632 | g-TGTATCACATTCTG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 633 | g-AAGAGGTAAGTCGT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 634 | -gcgcgcggcaatgc-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 635 | g-AAGGAGCCTAGCCG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 636 | g-ACGCGGTGCCCCAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| RUNX2 | 637 | g-CCTTACAGGAGTTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 638 | g-CACTATTACTGGAG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 639 | g-ACTGCCTACCACTG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 640 | g-TGTATCACATTCTG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 641 | g-AAGAGGTAAGTCGT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 642 | -gcgcgcggcaatgc-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 643 | g-AAGGAGCCTAGCCG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| RUNX2 | 644 | g-ACGCGGTGCCCCAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 645 | g-TAACCACACAATGA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 646 | g-ACTCTCTTGTATTC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 647 | g-AACAATGGCCAAGC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 648 | g-TTCTGTCTTCAATA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 649 | g-CTCTAGACAACCTC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 650 | -GACAAGTTGTTTAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 651 | g-AGAGAGCCTAGAAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 652 | -GGTCAAGGAGTCCT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 653 | g-TTGAGAGCAACACG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 654 | g-TCATGACCAGCAAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 655 | g-CGGTCACCGCGGCC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 656 | -GTGCAAATAACAAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX5 | 657 | g-TAACCACACAATGA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 658 | g-ACTCTCTTGTATTC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 659 | g-AACAATGGCCAAGC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 660 | g-TTCTGTCTTCAATA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 661 | g-CTCTAGACAACCTC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 662 | -GACAAGTTGTTTAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 663 | g-AGAGAGCCTAGAAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 664 | -GGTCAAGGAGTCCT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 665 | g-TTGAGAGCAACACG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 666 | g-TCATGACCAGCAAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 667 | g-CGGTCACCGCGGCC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 668 | -GTGCAAATAACAAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX5 | 669 | g-TAACCACACAATGA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 670 | g-ACTCTCTTGTATTC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 671 | g-AACAATGGCCAAGC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 672 | g-TTCTGTCTTCAATA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 673 | g-CTCTAGACAACCTC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 674 | -GACAAGTTGTTTAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 675 | g-AGAGAGCCTAGAAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 676 | -GGTCAAGGAGTCCT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 677 | g-TTGAGAGCAACACG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 678 | g-TCATGACCAGCAAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 679 | g-CGGTCACCGCGGCC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 680 | -GTGCAAATAACAAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX5 | 681 | g-TAACCACACAATGA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 682 | g-ACTCTCTTGTATTC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 683 | g-AACAATGGCCAAGC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 684 | g-TTCTGTCTTCAATA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 685 | g-CTCTAGACAACCTC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 686 | -GACAAGTTGTTTAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 687 | g-AGAGAGCCTAGAAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 688 | -GGTCAAGGAGTCCT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 689 | g-TTGAGAGCAACACG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 690 | g-TCATGACCAGCAAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 691 | g-CGGTCACCGCGGCC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 692 | -GTGCAAATAACAAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX5 | 693 | g-TAACCACACAATGA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 694 | g-ACTCTCTTGTATTC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 695 | g-AACAATGGCCAAGC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 696 | g-TTCTGTCTTCAATA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 697 | g-CTCTAGACAACCTC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 698 | -GACAAGTTGTTTAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 699 | g-AGAGAGCCTAGAAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 700 | -GGTCAAGGAGTCCT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 701 | g-TTGAGAGCAACACG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 702 | g-TCATGACCAGCAAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 703 | g-CGGTCACCGCGGCC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 704 | -GTGCAAATAACAAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX5 | 705 | g-TAACCACACAATGA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 706 | g-ACTCTCTTGTATTC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 707 | g-AACAATGGCCAAGC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 708 | g-TTCTGTCTTCAATA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 709 | g-CTCTAGACAACCTC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 710 | -GACAAGTTGTTTAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 711 | g-AGAGAGCCTAGAAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 712 | -GGTCAAGGAGTCCT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 713 | g-TTGAGAGCAACACG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 714 | g-TCATGACCAGCAAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 715 | g-CGGTCACCGCGGCC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 716 | -GTGCAAATAACAAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX5 | 717 | g-TAACCACACAATGA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 718 | g-ACTCTCTTGTATTC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 719 | g-AACAATGGCCAAGC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 720 | g-TTCTGTCTTCAATA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 721 | g-CTCTAGACAACCTC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 722 | -GACAAGTTGTTTAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 723 | g-AGAGAGCCTAGAAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 724 | -GGTCAAGGAGTCCT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 725 | g-TTGAGAGCAACACG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 726 | g-TCATGACCAGCAAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 727 | g-CGGTCACCGCGGCC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 728 | -GTGCAAATAACAAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX5 | 729 | g-TAACCACACAATGA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 730 | g-ACTCTCTTGTATTC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 731 | g-AACAATGGCCAAGC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 732 | g-TTCTGTCTTCAATA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 733 | g-CTCTAGACAACCTC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 734 | -GACAAGTTGTTTAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 735 | g-AGAGAGCCTAGAAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 736 | -GGTCAAGGAGTCCT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 737 | g-TTGAGAGCAACACG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 738 | g-TCATGACCAGCAAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 739 | g-CGGTCACCGCGGCC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 740 | -GTGCAAATAACAAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX5 | 741 | g-TAACCACACAATGA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 742 | g-ACTCTCTTGTATTC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 743 | g-AACAATGGCCAAGC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 744 | g-TTCTGTCTTCAATA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 745 | g-CTCTAGACAACCTC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 746 | -GACAAGTTGTTTAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 747 | g-AGAGAGCCTAGAAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 748 | -GGTCAAGGAGTCCT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 749 | g-TTGAGAGCAACACG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 750 | g-TCATGACCAGCAAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 751 | g-CGGTCACCGCGGCC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 752 | -GTGCAAATAACAAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX5 | 753 | g-TAACCACACAATGA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 754 | g-ACTCTCTTGTATTC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 755 | g-AACAATGGCCAAGC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 756 | g-TTCTGTCTTCAATA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 757 | g-CTCTAGACAACCTC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 758 | -GACAAGTTGTTTAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 759 | g-AGAGAGCCTAGAAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 760 | -GGTCAAGGAGTCCT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 761 | g-TTGAGAGCAACACG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 762 | g-TCATGACCAGCAAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 763 | g-CGGTCACCGCGGCC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 764 | -GTGCAAATAACAAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX5 | 765 | g-TAACCACACAATGA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 766 | g-ACTCTCTTGTATTC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 767 | g-AACAATGGCCAAGC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 768 | g-TTCTGTCTTCAATA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 769 | g-CTCTAGACAACCTC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 770 | -GACAAGTTGTTTAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 771 | g-AGAGAGCCTAGAAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 772 | -GGTCAAGGAGTCCT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 773 | g-TTGAGAGCAACACG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 774 | g-TCATGACCAGCAAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 775 | g-CGGTCACCGCGGCC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 776 | -GTGCAAATAACAAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX5 | 777 | g-TAACCACACAATGA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 778 | g-ACTCTCTTGTATTC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 779 | g-AACAATGGCCAAGC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 780 | g-TTCTGTCTTCAATA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 781 | g-CTCTAGACAACCTC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 782 | -GACAAGTTGTTTAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 783 | g-AGAGAGCCTAGAAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 784 | -GGTCAAGGAGTCCT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 785 | g-TTGAGAGCAACACG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 786 | g-TCATGACCAGCAAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 787 | g-CGGTCACCGCGGCC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 788 | -GTGCAAATAACAAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX5 | 789 | g-TAACCACACAATGA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 790 | g-ACTCTCTTGTATTC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 791 | g-AACAATGGCCAAGC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 792 | g-TTCTGTCTTCAATA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 793 | g-CTCTAGACAACCTC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 794 | -GACAAGTTGTTTAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 795 | g-AGAGAGCCTAGAAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 796 | -GGTCAAGGAGTCCT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 797 | g-TTGAGAGCAACACG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 798 | g-TCATGACCAGCAAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 799 | g-CGGTCACCGCGGCC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 800 | -GTGCAAATAACAAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX5 | 801 | g-TAACCACACAATGA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 802 | g-ACTCTCTTGTATTC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 803 | g-AACAATGGCCAAGC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 804 | g-TTCTGTCTTCAATA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 805 | g-CTCTAGACAACCTC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 806 | -GACAAGTTGTTTAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 807 | g-AGAGAGCCTAGAAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 808 | -GGTCAAGGAGTCCT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 809 | g-TTGAGAGCAACACG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 810 | g-TCATGACCAGCAAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 811 | g-CGGTCACCGCGGCC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 812 | -GTGCAAATAACAAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX5 | 813 | g-TAACCACACAATGA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 814 | g-ACTCTCTTGTATTC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 815 | g-AACAATGGCCAAGC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 816 | g-TTCTGTCTTCAATA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 817 | g-CTCTAGACAACCTC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 818 | -GACAAGTTGTTTAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 819 | g-AGAGAGCCTAGAAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 820 | -GGTCAAGGAGTCCT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 821 | g-TTGAGAGCAACACG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 822 | g-TCATGACCAGCAAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 823 | g-CGGTCACCGCGGCC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 824 | -GTGCAAATAACAAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX5 | 825 | g-TAACCACACAATGA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 826 | g-ACTCTCTTGTATTC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 827 | g-AACAATGGCCAAGC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 828 | g-TTCTGTCTTCAATA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 829 | g-CTCTAGACAACCTC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 830 | -GACAAGTTGTTTAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 831 | g-AGAGAGCCTAGAAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 832 | -GGTCAAGGAGTCCT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 833 | g-TTGAGAGCAACACG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 834 | g-TCATGACCAGCAAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 835 | g-CGGTCACCGCGGCC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 836 | -GTGCAAATAACAAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX5 | 837 | g-TAACCACACAATGA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 838 | g-ACTCTCTTGTATTC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 839 | g-AACAATGGCCAAGC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 840 | g-TTCTGTCTTCAATA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 841 | g-CTCTAGACAACCTC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 842 | -GACAAGTTGTTTAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 843 | g-AGAGAGCCTAGAAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 844 | -GGTCAAGGAGTCCT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 845 | g-TTGAGAGCAACACG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 846 | g-TCATGACCAGCAAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 847 | g-CGGTCACCGCGGCC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 848 | -GTGCAAATAACAAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX5 | 849 | g-TAACCACACAATGA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 850 | g-ACTCTCTTGTATTC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 851 | g-AACAATGGCCAAGC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 852 | g-TTCTGTCTTCAATA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 853 | g-CTCTAGACAACCTC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 854 | -GACAAGTTGTTTAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 855 | g-AGAGAGCCTAGAAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 856 | -GGTCAAGGAGTCCT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 857 | g-TTGAGAGCAACACG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 858 | g-TCATGACCAGCAAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 859 | g-CGGTCACCGCGGCC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 860 | -GTGCAAATAACAAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX5 | 861 | g-TAACCACACAATGA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 862 | g-ACTCTCTTGTATTC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 863 | g-AACAATGGCCAAGC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 864 | g-TTCTGTCTTCAATA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 865 | g-CTCTAGACAACCTC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 866 | -GACAAGTTGTTTAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 867 | g-AGAGAGCCTAGAAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 868 | -GGTCAAGGAGTCCT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 869 | g-TTGAGAGCAACACG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 870 | g-TCATGACCAGCAAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 871 | g-CGGTCACCGCGGCC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 872 | -GTGCAAATAACAAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX5 | 873 | g-TAACCACACAATGA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 874 | g-ACTCTCTTGTATTC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 875 | g-AACAATGGCCAAGC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 876 | g-TTCTGTCTTCAATA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 877 | g-CTCTAGACAACCTC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 878 | -GACAAGTTGTTTAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 879 | g-AGAGAGCCTAGAAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 880 | -GGTCAAGGAGTCCT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 881 | g-TTGAGAGCAACACG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 882 | g-TCATGACCAGCAAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 883 | g-CGGTCACCGCGGCC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 884 | -GTGCAAATAACAAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX5 | 885 | g-TAACCACACAATGA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 886 | g-ACTCTCTTGTATTC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 887 | g-AACAATGGCCAAGC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 888 | g-TTCTGTCTTCAATA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 889 | g-CTCTAGACAACCTC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 890 | -GACAAGTTGTTTAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 891 | g-AGAGAGCCTAGAAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 892 | -GGTCAAGGAGTCCT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 893 | g-TTGAGAGCAACACG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 894 | g-TCATGACCAGCAAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 895 | g-CGGTCACCGCGGCC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 896 | -GTGCAAATAACAAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX5 | 897 | g-TAACCACACAATGA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 898 | g-ACTCTCTTGTATTC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 899 | g-AACAATGGCCAAGC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 900 | g-TTCTGTCTTCAATA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 901 | g-CTCTAGACAACCTC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 902 | -GACAAGTTGTTTAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 903 | g-AGAGAGCCTAGAAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 904 | -GGTCAAGGAGTCCT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 905 | g-TTGAGAGCAACACG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 906 | g-TCATGACCAGCAAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 907 | g-CGGTCACCGCGGCC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 908 | -GTGCAAATAACAAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX5 | 909 | g-TAACCACACAATGA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 910 | g-ACTCTCTTGTATTC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 911 | g-AACAATGGCCAAGC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 912 | g-TTCTGTCTTCAATA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 913 | g-CTCTAGACAACCTC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 914 | -GACAAGTTGTTTAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 915 | g-AGAGAGCCTAGAAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 916 | -GGTCAAGGAGTCCT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 917 | g-TTGAGAGCAACACG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 918 | g-TCATGACCAGCAAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 919 | g-CGGTCACCGCGGCC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX5 | 920 | -GTGCAAATAACAAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 921 | g-CCAAATGCAAGGAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 922 | g-CCAATCCTTGCATT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 923 | -GTGATAGTTAGCAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 924 | g-TGAAAAAGAACTCC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 925 | g-ATAAGCTCAAAAGC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 926 | g-AGTTGGAGTTACTA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 927 | g-TTCAAGGACATGAA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 928 | g-AACTACACTCAGTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 929 | -GGGTTCCACGCCTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 930 | -GTTTGTTCTACCTA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 931 | -GAGCAAAACAATTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 932 | g-TAAATGTGCCTGAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 933 | g-TGATGTTGCACGAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 934 | g-TGAAGCAACTACTA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 935 | g-ATCGGCGAGACCAG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 936 | g-TGAGACAAGAGGCG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX6 | 937 | g-CCAAATGCAAGGAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 938 | g-CCAATCCTTGCATT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 939 | -GTGATAGTTAGCAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 940 | g-TGAAAAAGAACTCC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 941 | g-ATAAGCTCAAAAGC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 942 | g-AGTTGGAGTTACTA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 943 | g-TTCAAGGACATGAA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 944 | g-AACTACACTCAGTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 945 | -GGGTTCCACGCCTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 946 | -GTTTGTTCTACCTA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 947 | -GAGCAAAACAATTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 948 | g-TAAATGTGCCTGAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 949 | g-TGATGTTGCACGAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 950 | g-TGAAGCAACTACTA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 951 | g-ATCGGCGAGACCAG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 952 | g-TGAGACAAGAGGCG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX6 | 953 | g-CCAAATGCAAGGAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 954 | g-CCAATCCTTGCATT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 955 | -GTGATAGTTAGCAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 956 | g-TGAAAAAGAACTCC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 957 | g-ATAAGCTCAAAAGC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 958 | g-AGTTGGAGTTACTA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 959 | g-TTCAAGGACATGAA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 960 | g-AACTACACTCAGTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 961 | -GGGTTCCACGCCTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 962 | -GTTTGTTCTACCTA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 963 | -GAGCAAAACAATTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 964 | g-TAAATGTGCCTGAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 965 | g-TGATGTTGCACGAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 966 | g-TGAAGCAACTACTA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 967 | g-ATCGGCGAGACCAG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 968 | g-TGAGACAAGAGGCG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX6 | 969 | g-CCAAATGCAAGGAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 970 | g-CCAATCCTTGCATT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 971 | -GTGATAGTTAGCAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 972 | g-TGAAAAAGAACTCC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 973 | g-ATAAGCTCAAAAGC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 974 | g-AGTTGGAGTTACTA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 975 | g-TTCAAGGACATGAA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 976 | g-AACTACACTCAGTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 977 | -GGGTTCCACGCCTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 978 | -GTTTGTTCTACCTA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 979 | -GAGCAAAACAATTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 980 | g-TAAATGTGCCTGAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 981 | g-TGATGTTGCACGAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 982 | g-TGAAGCAACTACTA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 983 | g-ATCGGCGAGACCAG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 984 | g-TGAGACAAGAGGCG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX6 | 985 | g-CCAAATGCAAGGAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 986 | g-CCAATCCTTGCATT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 987 | -GTGATAGTTAGCAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 988 | g-TGAAAAAGAACTCC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 989 | g-ATAAGCTCAAAAGC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 990 | g-AGTTGGAGTTACTA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 991 | g-TTCAAGGACATGAA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 992 | g-AACTACACTCAGTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 993 | -GGGTTCCACGCCTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 994 | -GTTTGTTCTACCTA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 995 | -GAGCAAAACAATTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 996 | g-TAAATGTGCCTGAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 997 | g-TGATGTTGCACGAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 998 | g-TGAAGCAACTACTA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 999 | g-ATCGGCGAGACCAG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 1000 | g-TGAGACAAGAGGCG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX6 | 1001 | g-CCAAATGCAAGGAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1002 | g-CCAATCCTTGCATT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1003 | -GTGATAGTTAGCAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1004 | g-TGAAAAAGAACTCC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1005 | g-ATAAGCTCAAAAGC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1006 | g-AGTTGGAGTTACTA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1007 | g-TTCAAGGACATGAA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1008 | g-AACTACACTCAGTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1009 | -GGGTTCCACGCCTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1010 | -GTTTGTTCTACCTA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1011 | -GAGCAAAACAATTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1012 | g-TAAATGTGCCTGAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1013 | g-TGATGTTGCACGAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1014 | g-TGAAGCAACTACTA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1015 | g-ATCGGCGAGACCAG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1016 | g-TGAGACAAGAGGCG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX6 | 1017 | g-CCAAATGCAAGGAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1018 | g-CCAATCCTTGCATT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1019 | -GTGATAGTTAGCAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1020 | g-TGAAAAAGAACTCC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1021 | g-ATAAGCTCAAAAGC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1022 | g-AGTTGGAGTTACTA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1023 | g-TTCAAGGACATGAA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1024 | g-AACTACACTCAGTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1025 | -GGGTTCCACGCCTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1026 | -GTTTGTTCTACCTA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1027 | -GAGCAAAACAATTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1028 | g-TAAATGTGCCTGAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1029 | g-TGATGTTGCACGAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1030 | g-TGAAGCAACTACTA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1031 | g-ATCGGCGAGACCAG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1032 | g-TGAGACAAGAGGCG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX6 | 1033 | g-CCAAATGCAAGGAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1034 | g-CCAATCCTTGCATT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1035 | -GTGATAGTTAGCAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1036 | g-TGAAAAAGAACTCC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1037 | g-ATAAGCTCAAAAGC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1038 | g-AGTTGGAGTTACTA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1039 | g-TTCAAGGACATGAA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1040 | g-AACTACACTCAGTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1041 | -GGGTTCCACGCCTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1042 | -GTTTGTTCTACCTA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1043 | -GAGCAAAACAATTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1044 | g-TAAATGTGCCTGAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1045 | g-TGATGTTGCACGAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1046 | g-TGAAGCAACTACTA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1047 | g-ATCGGCGAGACCAG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1048 | g-TGAGACAAGAGGCG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX6 | 1049 | g-CCAAATGCAAGGAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1050 | g-CCAATCCTTGCATT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1051 | -GTGATAGTTAGCAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1052 | g-TGAAAAAGAACTCC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1053 | g-ATAAGCTCAAAAGC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1054 | g-AGTTGGAGTTACTA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1055 | g-TTCAAGGACATGAA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1056 | g-AACTACACTCAGTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1057 | -GGGTTCCACGCCTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1058 | -GTTTGTTCTACCTA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1059 | -GAGCAAAACAATTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1060 | g-TAAATGTGCCTGAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1061 | g-TGATGTTGCACGAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1062 | g-TGAAGCAACTACTA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1063 | g-ATCGGCGAGACCAG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1064 | g-TGAGACAAGAGGCG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX6 | 1065 | g-CCAAATGCAAGGAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1066 | g-CCAATCCTTGCATT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1067 | -GTGATAGTTAGCAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1068 | g-TGAAAAAGAACTCC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1069 | g-ATAAGCTCAAAAGC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1070 | g-AGTTGGAGTTACTA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1071 | g-TTCAAGGACATGAA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1072 | g-AACTACACTCAGTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1073 | -GGGTTCCACGCCTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1074 | -GTTTGTTCTACCTA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1075 | -GAGCAAAACAATTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1076 | g-TAAATGTGCCTGAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1077 | g-TGATGTTGCACGAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1078 | g-TGAAGCAACTACTA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1079 | g-ATCGGCGAGACCAG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1080 | g-TGAGACAAGAGGCG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX6 | 1081 | g-CCAAATGCAAGGAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1082 | g-CCAATCCTTGCATT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1083 | -GTGATAGTTAGCAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1084 | g-TGAAAAAGAACTCC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1085 | g-ATAAGCTCAAAAGC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1086 | g-AGTTGGAGTTACTA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1087 | g-TTCAAGGACATGAA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1088 | g-AACTACACTCAGTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1089 | -GGGTTCCACGCCTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1090 | -GTTTGTTCTACCTA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1091 | -GAGCAAAACAATTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1092 | g-TAAATGTGCCTGAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1093 | g-TGATGTTGCACGAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1094 | g-TGAAGCAACTACTA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1095 | g-ATCGGCGAGACCAG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1096 | g-TGAGACAAGAGGCG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX6 | 1097 | g-CCAAATGCAAGGAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1098 | g-CCAATCCTTGCATT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1099 | -GTGATAGTTAGCAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1100 | g-TGAAAAAGAACTCC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1101 | g-ATAAGCTCAAAAGC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1102 | g-AGTTGGAGTTACTA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1103 | g-TTCAAGGACATGAA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1104 | g-AACTACACTCAGTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1105 | -GGGTTCCACGCCTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1106 | -GTTTGTTCTACCTA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1107 | -GAGCAAAACAATTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1108 | g-TAAATGTGCCTGAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1109 | g-TGATGTTGCACGAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1110 | g-TGAAGCAACTACTA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1111 | g-ATCGGCGAGACCAG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1112 | g-TGAGACAAGAGGCG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX6 | 1113 | g-CCAAATGCAAGGAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1114 | g-CCAATCCTTGCATT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1115 | -GTGATAGTTAGCAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1116 | g-TGAAAAAGAACTCC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1117 | g-ATAAGCTCAAAAGC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1118 | g-AGTTGGAGTTACTA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1119 | g-TTCAAGGACATGAA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1120 | g-AACTACACTCAGTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1121 | -GGGTTCCACGCCTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1122 | -GTTTGTTCTACCTA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1123 | -GAGCAAAACAATTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1124 | g-TAAATGTGCCTGAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1125 | g-TGATGTTGCACGAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1126 | g-TGAAGCAACTACTA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1127 | g-ATCGGCGAGACCAG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1128 | g-TGAGACAAGAGGCG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX6 | 1129 | g-CCAAATGCAAGGAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1130 | g-CCAATCCTTGCATT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1131 | -GTGATAGTTAGCAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1132 | g-TGAAAAAGAACTCC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1133 | g-ATAAGCTCAAAAGC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1134 | g-AGTTGGAGTTACTA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1135 | g-TTCAAGGACATGAA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1136 | g-AACTACACTCAGTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1137 | -GGGTTCCACGCCTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1138 | -GTTTGTTCTACCTA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1139 | -GAGCAAAACAATTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1140 | g-TAAATGTGCCTGAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1141 | g-TGATGTTGCACGAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1142 | g-TGAAGCAACTACTA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1143 | g-ATCGGCGAGACCAG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1144 | g-TGAGACAAGAGGCG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX6 | 1145 | g-CCAAATGCAAGGAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1146 | g-CCAATCCTTGCATT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1147 | -GTGATAGTTAGCAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1148 | g-TGAAAAAGAACTCC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1149 | g-ATAAGCTCAAAAGC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1150 | g-AGTTGGAGTTACTA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1151 | g-TTCAAGGACATGAA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1152 | g-AACTACACTCAGTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1153 | -GGGTTCCACGCCTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1154 | -GTTTGTTCTACCTA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1155 | -GAGCAAAACAATTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1156 | g-TAAATGTGCCTGAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1157 | g-TGATGTTGCACGAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1158 | g-TGAAGCAACTACTA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1159 | g-ATCGGCGAGACCAG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1160 | g-TGAGACAAGAGGCG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX6 | 1161 | g-CCAAATGCAAGGAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1162 | g-CCAATCCTTGCATT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1163 | -GTGATAGTTAGCAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1164 | g-TGAAAAAGAACTCC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1165 | g-ATAAGCTCAAAAGC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1166 | g-AGTTGGAGTTACTA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1167 | g-TTCAAGGACATGAA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1168 | g-AACTACACTCAGTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1169 | -GGGTTCCACGCCTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1170 | -GTTTGTTCTACCTA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1171 | -GAGCAAAACAATTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1172 | g-TAAATGTGCCTGAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1173 | g-TGATGTTGCACGAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1174 | g-TGAAGCAACTACTA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1175 | g-ATCGGCGAGACCAG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1176 | g-TGAGACAAGAGGCG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX6 | 1177 | g-CCAAATGCAAGGAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1178 | g-CCAATCCTTGCATT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1179 | -GTGATAGTTAGCAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1180 | g-TGAAAAAGAACTCC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1181 | g-ATAAGCTCAAAAGC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1182 | g-AGTTGGAGTTACTA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1183 | g-TTCAAGGACATGAA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1184 | g-AACTACACTCAGTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1185 | -GGGTTCCACGCCTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1186 | -GTTTGTTCTACCTA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1187 | -GAGCAAAACAATTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1188 | g-TAAATGTGCCTGAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1189 | g-TGATGTTGCACGAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1190 | g-TGAAGCAACTACTA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1191 | g-ATCGGCGAGACCAG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1192 | g-TGAGACAAGAGGCG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX6 | 1193 | g-CCAAATGCAAGGAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1194 | g-CCAATCCTTGCATT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1195 | -GTGATAGTTAGCAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1196 | g-TGAAAAAGAACTCC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1197 | g-ATAAGCTCAAAAGC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1198 | g-AGTTGGAGTTACTA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1199 | g-TTCAAGGACATGAA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1200 | g-AACTACACTCAGTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1201 | -GGGTTCCACGCCTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1202 | -GTTTGTTCTACCTA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1203 | -GAGCAAAACAATTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1204 | g-TAAATGTGCCTGAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1205 | g-TGATGTTGCACGAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1206 | g-TGAAGCAACTACTA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1207 | g-ATCGGCGAGACCAG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1208 | g-TGAGACAAGAGGCG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX6 | 1209 | g-CCAAATGCAAGGAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1210 | g-CCAATCCTTGCATT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1211 | -GTGATAGTTAGCAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1212 | g-TGAAAAAGAACTCC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1213 | g-ATAAGCTCAAAAGC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1214 | g-AGTTGGAGTTACTA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1215 | g-TTCAAGGACATGAA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1216 | g-AACTACACTCAGTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1217 | -GGGTTCCACGCCTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1218 | -GTTTGTTCTACCTA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1219 | -GAGCAAAACAATTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1220 | g-TAAATGTGCCTGAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1221 | g-TGATGTTGCACGAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1222 | g-TGAAGCAACTACTA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1223 | g-ATCGGCGAGACCAG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1224 | g-TGAGACAAGAGGCG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX6 | 1225 | g-CCAAATGCAAGGAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1226 | g-CCAATCCTTGCATT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1227 | -GTGATAGTTAGCAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1228 | g-TGAAAAAGAACTCC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1229 | g-ATAAGCTCAAAAGC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1230 | g-AGTTGGAGTTACTA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1231 | g-TTCAAGGACATGAA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1232 | g-AACTACACTCAGTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1233 | -GGGTTCCACGCCTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1234 | -GTTTGTTCTACCTA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1235 | -GAGCAAAACAATTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1236 | g-TAAATGTGCCTGAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1237 | g-TGATGTTGCACGAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1238 | g-TGAAGCAACTACTA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1239 | g-ATCGGCGAGACCAG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1240 | g-TGAGACAAGAGGCG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX6 | 1241 | g-CCAAATGCAAGGAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1242 | g-CCAATCCTTGCATT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1243 | -GTGATAGTTAGCAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1244 | g-TGAAAAAGAACTCC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1245 | g-ATAAGCTCAAAAGC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1246 | g-AGTTGGAGTTACTA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1247 | g-TTCAAGGACATGAA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1248 | g-AACTACACTCAGTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1249 | -GGGTTCCACGCCTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1250 | -GTTTGTTCTACCTA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1251 | -GAGCAAAACAATTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1252 | g-TAAATGTGCCTGAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1253 | g-TGATGTTGCACGAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1254 | g-TGAAGCAACTACTA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1255 | g-ATCGGCGAGACCAG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1256 | g-TGAGACAAGAGGCG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX6 | 1257 | g-CCAAATGCAAGGAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1258 | g-CCAATCCTTGCATT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1259 | -GTGATAGTTAGCAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1260 | g-TGAAAAAGAACTCC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1261 | g-ATAAGCTCAAAAGC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1262 | g-AGTTGGAGTTACTA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1263 | g-TTCAAGGACATGAA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1264 | g-AACTACACTCAGTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1265 | -GGGTTCCACGCCTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1266 | -GTTTGTTCTACCTA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1267 | -GAGCAAAACAATTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1268 | g-TAAATGTGCCTGAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1269 | g-TGATGTTGCACGAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1270 | g-TGAAGCAACTACTA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1271 | g-ATCGGCGAGACCAG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1272 | g-TGAGACAAGAGGCG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX6 | 1273 | g-CCAAATGCAAGGAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1274 | g-CCAATCCTTGCATT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1275 | -GTGATAGTTAGCAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1276 | g-TGAAAAAGAACTCC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1277 | g-ATAAGCTCAAAAGC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1278 | g-AGTTGGAGTTACTA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1279 | g-TTCAAGGACATGAA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1280 | g-AACTACACTCAGTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1281 | -GGGTTCCACGCCTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1282 | -GTTTGTTCTACCTA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1283 | -GAGCAAAACAATTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1284 | g-TAAATGTGCCTGAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1285 | g-TGATGTTGCACGAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1286 | g-TGAAGCAACTACTA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1287 | g-ATCGGCGAGACCAG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX6 | 1288 | g-TGAGACAAGAGGCG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX8 | 1289 | g-CTCAGGCGCGAGAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX8 | 1290 | -GCTTTCTTTATGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX8 | 1291 | g-AGGTCGCCGCGCGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX8 | 1292 | -gcccggcTACCCAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX8 | 1293 | g-CTCAGGCGCGAGAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX8 | 1294 | -GCTTTCTTTATGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX8 | 1295 | g-AGGTCGCCGCGCGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX8 | 1296 | -gcccggcTACCCAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX8 | 1297 | g-CTCAGGCGCGAGAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX8 | 1298 | -GCTTTCTTTATGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX8 | 1299 | g-AGGTCGCCGCGCGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX8 | 1300 | -gcccggcTACCCAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX8 | 1301 | g-CTCAGGCGCGAGAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX8 | 1302 | -GCTTTCTTTATGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX8 | 1303 | g-AGGTCGCCGCGCGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX8 | 1304 | -gcccggcTACCCAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX8 | 1305 | g-CTCAGGCGCGAGAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX8 | 1306 | -GCTTTCTTTATGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX8 | 1307 | g-AGGTCGCCGCGCGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX8 | 1308 | -gcccggcTACCCAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX8 | 1309 | g-CTCAGGCGCGAGAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX8 | 1310 | -GCTTTCTTTATGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX8 | 1311 | g-AGGTCGCCGCGCGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX8 | 1312 | -gcccggcTACCCAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX8 | 1313 | g-CTCAGGCGCGAGAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX8 | 1314 | -GCTTTCTTTATGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX8 | 1315 | g-AGGTCGCCGCGCGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX8 | 1316 | -gcccggcTACCCAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX8 | 1317 | g-CTCAGGCGCGAGAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX8 | 1318 | -GCTTTCTTTATGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX8 | 1319 | g-AGGTCGCCGCGCGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX8 | 1320 | -gcccggcTACCCAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX8 | 1321 | g-CTCAGGCGCGAGAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX8 | 1322 | -GCTTTCTTTATGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX8 | 1323 | g-AGGTCGCCGCGCGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX8 | 1324 | -gcccggcTACCCAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX8 | 1325 | g-CTCAGGCGCGAGAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX8 | 1326 | -GCTTTCTTTATGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX8 | 1327 | g-AGGTCGCCGCGCGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX8 | 1328 | -gcccggcTACCCAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX8 | 1329 | g-CTCAGGCGCGAGAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX8 | 1330 | -GCTTTCTTTATGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX8 | 1331 | g-AGGTCGCCGCGCGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX8 | 1332 | -gcccggcTACCCAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX8 | 1333 | g-CTCAGGCGCGAGAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX8 | 1334 | -GCTTTCTTTATGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX8 | 1335 | g-AGGTCGCCGCGCGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX8 | 1336 | -gcccggcTACCCAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX8 | 1337 | g-CTCAGGCGCGAGAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX8 | 1338 | -GCTTTCTTTATGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX8 | 1339 | g-AGGTCGCCGCGCGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX8 | 1340 | -gcccggcTACCCAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX8 | 1341 | g-CTCAGGCGCGAGAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX8 | 1342 | -GCTTTCTTTATGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX8 | 1343 | g-AGGTCGCCGCGCGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX8 | 1344 | -gcccggcTACCCAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX8 | 1345 | g-CTCAGGCGCGAGAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX8 | 1346 | -GCTTTCTTTATGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX8 | 1347 | g-AGGTCGCCGCGCGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX8 | 1348 | -gcccggcTACCCAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX8 | 1349 | g-CTCAGGCGCGAGAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX8 | 1350 | -GCTTTCTTTATGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX8 | 1351 | g-AGGTCGCCGCGCGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX8 | 1352 | -gcccggcTACCCAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX8 | 1353 | g-CTCAGGCGCGAGAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX8 | 1354 | -GCTTTCTTTATGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX8 | 1355 | g-AGGTCGCCGCGCGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX8 | 1356 | -gcccggcTACCCAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX8 | 1357 | g-CTCAGGCGCGAGAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX8 | 1358 | -GCTTTCTTTATGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX8 | 1359 | g-AGGTCGCCGCGCGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX8 | 1360 | -gcccggcTACCCAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX8 | 1361 | g-CTCAGGCGCGAGAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX8 | 1362 | -GCTTTCTTTATGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX8 | 1363 | g-AGGTCGCCGCGCGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX8 | 1364 | -gcccggcTACCCAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX8 | 1365 | g-CTCAGGCGCGAGAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX8 | 1366 | -GCTTTCTTTATGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX8 | 1367 | g-AGGTCGCCGCGCGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX8 | 1368 | -gcccggcTACCCAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX8 | 1369 | g-CTCAGGCGCGAGAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX8 | 1370 | -GCTTTCTTTATGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX8 | 1371 | g-AGGTCGCCGCGCGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX8 | 1372 | -gcccggcTACCCAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX8 | 1373 | g-CTCAGGCGCGAGAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX8 | 1374 | -GCTTTCTTTATGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX8 | 1375 | g-AGGTCGCCGCGCGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX8 | 1376 | -gcccggcTACCCAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX8 | 1377 | g-CTCAGGCGCGAGAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX8 | 1378 | -GCTTTCTTTATGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX8 | 1379 | g-AGGTCGCCGCGCGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX8 | 1380 | -gcccggcTACCCAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX9 | 1381 | g-CAAACTTACACACT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX9 | 1382 | g-AAAGGGCGGACGGT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX9 | 1383 | g-ATTGGACCCGATTT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX9 | 1384 | -GTGGCTCTAAGGTG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SOX9 | 1385 | g-CAAACTTACACACT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX9 | 1386 | g-AAAGGGCGGACGGT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX9 | 1387 | g-ATTGGACCCGATTT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX9 | 1388 | -GTGGCTCTAAGGTG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SOX9 | 1389 | g-CAAACTTACACACT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX9 | 1390 | g-AAAGGGCGGACGGT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX9 | 1391 | g-ATTGGACCCGATTT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX9 | 1392 | -GTGGCTCTAAGGTG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SOX9 | 1393 | g-CAAACTTACACACT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX9 | 1394 | g-AAAGGGCGGACGGT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX9 | 1395 | g-ATTGGACCCGATTT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX9 | 1396 | -GTGGCTCTAAGGTG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SOX9 | 1397 | g-CAAACTTACACACT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX9 | 1398 | g-AAAGGGCGGACGGT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX9 | 1399 | g-ATTGGACCCGATTT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX9 | 1400 | -GTGGCTCTAAGGTG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SOX9 | 1401 | g-CAAACTTACACACT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX9 | 1402 | g-AAAGGGCGGACGGT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX9 | 1403 | g-ATTGGACCCGATTT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX9 | 1404 | -GTGGCTCTAAGGTG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SOX9 | 1405 | g-CAAACTTACACACT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX9 | 1406 | g-AAAGGGCGGACGGT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX9 | 1407 | g-ATTGGACCCGATTT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX9 | 1408 | -GTGGCTCTAAGGTG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SOX9 | 1409 | g-CAAACTTACACACT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX9 | 1410 | g-AAAGGGCGGACGGT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX9 | 1411 | g-ATTGGACCCGATTT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX9 | 1412 | -GTGGCTCTAAGGTG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SOX9 | 1413 | g-CAAACTTACACACT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX9 | 1414 | g-AAAGGGCGGACGGT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX9 | 1415 | g-ATTGGACCCGATTT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX9 | 1416 | -GTGGCTCTAAGGTG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SOX9 | 1417 | g-CAAACTTACACACT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX9 | 1418 | g-AAAGGGCGGACGGT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX9 | 1419 | g-ATTGGACCCGATTT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX9 | 1420 | -GTGGCTCTAAGGTG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SOX9 | 1421 | g-CAAACTTACACACT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX9 | 1422 | g-AAAGGGCGGACGGT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX9 | 1423 | g-ATTGGACCCGATTT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX9 | 1424 | -GTGGCTCTAAGGTG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SOX9 | 1425 | g-CAAACTTACACACT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX9 | 1426 | g-AAAGGGCGGACGGT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX9 | 1427 | g-ATTGGACCCGATTT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX9 | 1428 | -GTGGCTCTAAGGTG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SOX9 | 1429 | g-CAAACTTACACACT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX9 | 1430 | g-AAAGGGCGGACGGT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX9 | 1431 | g-ATTGGACCCGATTT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX9 | 1432 | -GTGGCTCTAAGGTG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SOX9 | 1433 | g-CAAACTTACACACT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX9 | 1434 | g-AAAGGGCGGACGGT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX9 | 1435 | g-ATTGGACCCGATTT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX9 | 1436 | -GTGGCTCTAAGGTG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SOX9 | 1437 | g-CAAACTTACACACT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX9 | 1438 | g-AAAGGGCGGACGGT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX9 | 1439 | g-ATTGGACCCGATTT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX9 | 1440 | -GTGGCTCTAAGGTG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SOX9 | 1441 | g-CAAACTTACACACT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX9 | 1442 | g-AAAGGGCGGACGGT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX9 | 1443 | g-ATTGGACCCGATTT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX9 | 1444 | -GTGGCTCTAAGGTG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SOX9 | 1445 | g-CAAACTTACACACT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX9 | 1446 | g-AAAGGGCGGACGGT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX9 | 1447 | g-ATTGGACCCGATTT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX9 | 1448 | -GTGGCTCTAAGGTG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SOX9 | 1449 | g-CAAACTTACACACT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX9 | 1450 | g-AAAGGGCGGACGGT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX9 | 1451 | g-ATTGGACCCGATTT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX9 | 1452 | -GTGGCTCTAAGGTG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SOX9 | 1453 | g-CAAACTTACACACT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX9 | 1454 | g-AAAGGGCGGACGGT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX9 | 1455 | g-ATTGGACCCGATTT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX9 | 1456 | -GTGGCTCTAAGGTG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SOX9 | 1457 | g-CAAACTTACACACT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX9 | 1458 | g-AAAGGGCGGACGGT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX9 | 1459 | g-ATTGGACCCGATTT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX9 | 1460 | -GTGGCTCTAAGGTG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SOX9 | 1461 | g-CAAACTTACACACT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX9 | 1462 | g-AAAGGGCGGACGGT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX9 | 1463 | g-ATTGGACCCGATTT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX9 | 1464 | -GTGGCTCTAAGGTG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SOX9 | 1465 | g-CAAACTTACACACT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX9 | 1466 | g-AAAGGGCGGACGGT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX9 | 1467 | g-ATTGGACCCGATTT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX9 | 1468 | -GTGGCTCTAAGGTG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SOX9 | 1469 | g-CAAACTTACACACT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX9 | 1470 | g-AAAGGGCGGACGGT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX9 | 1471 | g-ATTGGACCCGATTT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SOX9 | 1472 | -GTGGCTCTAAGGTG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SP1 | 1473 | g-TGAGACGTAGGGAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SP1 | 1474 | -GCGAGTCTTGCCAT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SP1 | 1475 | -GGACCGGACAGGGA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SP1 | 1476 | g-CCGCCCAATGAGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| SP1 | 1477 | g-TGAGACGTAGGGAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SP1 | 1478 | -GCGAGTCTTGCCAT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SP1 | 1479 | -GGACCGGACAGGGA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SP1 | 1480 | g-CCGCCCAATGAGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| SP1 | 1481 | g-TGAGACGTAGGGAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SP1 | 1482 | -GCGAGTCTTGCCAT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SP1 | 1483 | -GGACCGGACAGGGA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SP1 | 1484 | g-CCGCCCAATGAGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| SP1 | 1485 | g-TGAGACGTAGGGAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SP1 | 1486 | -GCGAGTCTTGCCAT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SP1 | 1487 | -GGACCGGACAGGGA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SP1 | 1488 | g-CCGCCCAATGAGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| SP1 | 1489 | g-TGAGACGTAGGGAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SP1 | 1490 | -GCGAGTCTTGCCAT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SP1 | 1491 | -GGACCGGACAGGGA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SP1 | 1492 | g-CCGCCCAATGAGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| SP1 | 1493 | g-TGAGACGTAGGGAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SP1 | 1494 | -GCGAGTCTTGCCAT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SP1 | 1495 | -GGACCGGACAGGGA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SP1 | 1496 | g-CCGCCCAATGAGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| SP1 | 1497 | g-TGAGACGTAGGGAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SP1 | 1498 | -GCGAGTCTTGCCAT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SP1 | 1499 | -GGACCGGACAGGGA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SP1 | 1500 | g-CCGCCCAATGAGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| SP1 | 1501 | g-TGAGACGTAGGGAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SP1 | 1502 | -GCGAGTCTTGCCAT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SP1 | 1503 | -GGACCGGACAGGGA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SP1 | 1504 | g-CCGCCCAATGAGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| SP1 | 1505 | g-TGAGACGTAGGGAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SP1 | 1506 | -GCGAGTCTTGCCAT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SP1 | 1507 | -GGACCGGACAGGGA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SP1 | 1508 | g-CCGCCCAATGAGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| SP1 | 1509 | g-TGAGACGTAGGGAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SP1 | 1510 | -GCGAGTCTTGCCAT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SP1 | 1511 | -GGACCGGACAGGGA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SP1 | 1512 | g-CCGCCCAATGAGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| SP1 | 1513 | g-TGAGACGTAGGGAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SP1 | 1514 | -GCGAGTCTTGCCAT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SP1 | 1515 | -GGACCGGACAGGGA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SP1 | 1516 | g-CCGCCCAATGAGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| SP1 | 1517 | g-TGAGACGTAGGGAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SP1 | 1518 | -GCGAGTCTTGCCAT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SP1 | 1519 | -GGACCGGACAGGGA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SP1 | 1520 | g-CCGCCCAATGAGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| SP1 | 1521 | g-TGAGACGTAGGGAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SP1 | 1522 | -GCGAGTCTTGCCAT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SP1 | 1523 | -GGACCGGACAGGGA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SP1 | 1524 | g-CCGCCCAATGAGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| SP1 | 1525 | g-TGAGACGTAGGGAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SP1 | 1526 | -GCGAGTCTTGCCAT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SP1 | 1527 | -GGACCGGACAGGGA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SP1 | 1528 | g-CCGCCCAATGAGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| SP1 | 1529 | g-TGAGACGTAGGGAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SP1 | 1530 | -GCGAGTCTTGCCAT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SP1 | 1531 | -GGACCGGACAGGGA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SP1 | 1532 | g-CCGCCCAATGAGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| SP1 | 1533 | g-TGAGACGTAGGGAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SP1 | 1534 | -GCGAGTCTTGCCAT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SP1 | 1535 | -GGACCGGACAGGGA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SP1 | 1536 | g-CCGCCCAATGAGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| SP1 | 1537 | g-TGAGACGTAGGGAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SP1 | 1538 | -GCGAGTCTTGCCAT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SP1 | 1539 | -GGACCGGACAGGGA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SP1 | 1540 | g-CCGCCCAATGAGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| SP1 | 1541 | g-TGAGACGTAGGGAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SP1 | 1542 | -GCGAGTCTTGCCAT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SP1 | 1543 | -GGACCGGACAGGGA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SP1 | 1544 | g-CCGCCCAATGAGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| SP1 | 1545 | g-TGAGACGTAGGGAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SP1 | 1546 | -GCGAGTCTTGCCAT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SP1 | 1547 | -GGACCGGACAGGGA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SP1 | 1548 | g-CCGCCCAATGAGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| SP1 | 1549 | g-TGAGACGTAGGGAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SP1 | 1550 | -GCGAGTCTTGCCAT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SP1 | 1551 | -GGACCGGACAGGGA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SP1 | 1552 | g-CCGCCCAATGAGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| SP1 | 1553 | g-TGAGACGTAGGGAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SP1 | 1554 | -GCGAGTCTTGCCAT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SP1 | 1555 | -GGACCGGACAGGGA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SP1 | 1556 | g-CCGCCCAATGAGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| SP1 | 1557 | g-TGAGACGTAGGGAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SP1 | 1558 | -GCGAGTCTTGCCAT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SP1 | 1559 | -GGACCGGACAGGGA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SP1 | 1560 | g-CCGCCCAATGAGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| SP1 | 1561 | g-TGAGACGTAGGGAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SP1 | 1562 | -GCGAGTCTTGCCAT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SP1 | 1563 | -GGACCGGACAGGGA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| SP1 | 1564 | g-CCGCCCAATGAGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF15 | 1565 | g-AAAAGTTCGGATCT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF15 | 1566 | -GTCGCCATTGGCCA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF15 | 1567 | g-CTCCCTCGCCTATA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF15 | 1568 | -GGCCCGGGAGCCGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF15 | 1569 | g-AAAAGTTCGGATCT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF15 | 1570 | -GTCGCCATTGGCCA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF15 | 1571 | g-CTCCCTCGCCTATA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF15 | 1572 | -GGCCCGGGAGCCGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF15 | 1573 | g-AAAAGTTCGGATCT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF15 | 1574 | -GTCGCCATTGGCCA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF15 | 1575 | g-CTCCCTCGCCTATA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF15 | 1576 | -GGCCCGGGAGCCGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF15 | 1577 | g-AAAAGTTCGGATCT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF15 | 1578 | -GTCGCCATTGGCCA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF15 | 1579 | g-CTCCCTCGCCTATA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF15 | 1580 | -GGCCCGGGAGCCGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF15 | 1581 | g-AAAAGTTCGGATCT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF15 | 1582 | -GTCGCCATTGGCCA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF15 | 1583 | g-CTCCCTCGCCTATA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF15 | 1584 | -GGCCCGGGAGCCGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF15 | 1585 | g-AAAAGTTCGGATCT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF15 | 1586 | -GTCGCCATTGGCCA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF15 | 1587 | g-CTCCCTCGCCTATA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF15 | 1588 | -GGCCCGGGAGCCGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF15 | 1589 | g-AAAAGTTCGGATCT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF15 | 1590 | -GTCGCCATTGGCCA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF15 | 1591 | g-CTCCCTCGCCTATA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF15 | 1592 | -GGCCCGGGAGCCGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF15 | 1593 | g-AAAAGTTCGGATCT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF15 | 1594 | -GTCGCCATTGGCCA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF15 | 1595 | g-CTCCCTCGCCTATA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF15 | 1596 | -GGCCCGGGAGCCGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF15 | 1597 | g-AAAAGTTCGGATCT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF15 | 1598 | -GTCGCCATTGGCCA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF15 | 1599 | g-CTCCCTCGCCTATA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF15 | 1600 | -GGCCCGGGAGCCGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF15 | 1601 | g-AAAAGTTCGGATCT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF15 | 1602 | -GTCGCCATTGGCCA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF15 | 1603 | g-CTCCCTCGCCTATA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF15 | 1604 | -GGCCCGGGAGCCGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF15 | 1605 | g-AAAAGTTCGGATCT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF15 | 1606 | -GTCGCCATTGGCCA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF15 | 1607 | g-CTCCCTCGCCTATA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF15 | 1608 | -GGCCCGGGAGCCGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF15 | 1609 | g-AAAAGTTCGGATCT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF15 | 1610 | -GTCGCCATTGGCCA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF15 | 1611 | g-CTCCCTCGCCTATA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF15 | 1612 | -GGCCCGGGAGCCGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF15 | 1613 | g-AAAAGTTCGGATCT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF15 | 1614 | -GTCGCCATTGGCCA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF15 | 1615 | g-CTCCCTCGCCTATA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF15 | 1616 | -GGCCCGGGAGCCGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF15 | 1617 | g-AAAAGTTCGGATCT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF15 | 1618 | -GTCGCCATTGGCCA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF15 | 1619 | g-CTCCCTCGCCTATA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF15 | 1620 | -GGCCCGGGAGCCGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF15 | 1621 | g-AAAAGTTCGGATCT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF15 | 1622 | -GTCGCCATTGGCCA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF15 | 1623 | g-CTCCCTCGCCTATA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF15 | 1624 | -GGCCCGGGAGCCGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF15 | 1625 | g-AAAAGTTCGGATCT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF15 | 1626 | -GTCGCCATTGGCCA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF15 | 1627 | g-CTCCCTCGCCTATA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF15 | 1628 | -GGCCCGGGAGCCGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF15 | 1629 | g-AAAAGTTCGGATCT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF15 | 1630 | -GTCGCCATTGGCCA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF15 | 1631 | g-CTCCCTCGCCTATA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF15 | 1632 | -GGCCCGGGAGCCGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF15 | 1633 | g-AAAAGTTCGGATCT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF15 | 1634 | -GTCGCCATTGGCCA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF15 | 1635 | g-CTCCCTCGCCTATA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF15 | 1636 | -GGCCCGGGAGCCGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF15 | 1637 | g-AAAAGTTCGGATCT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF15 | 1638 | -GTCGCCATTGGCCA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF15 | 1639 | g-CTCCCTCGCCTATA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF15 | 1640 | -GGCCCGGGAGCCGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF15 | 1641 | g-AAAAGTTCGGATCT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF15 | 1642 | -GTCGCCATTGGCCA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF15 | 1643 | g-CTCCCTCGCCTATA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF15 | 1644 | -GGCCCGGGAGCCGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF15 | 1645 | g-AAAAGTTCGGATCT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF15 | 1646 | -GTCGCCATTGGCCA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF15 | 1647 | g-CTCCCTCGCCTATA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF15 | 1648 | -GGCCCGGGAGCCGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF15 | 1649 | g-AAAAGTTCGGATCT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF15 | 1650 | -GTCGCCATTGGCCA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF15 | 1651 | g-CTCCCTCGCCTATA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF15 | 1652 | -GGCCCGGGAGCCGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF15 | 1653 | g-AAAAGTTCGGATCT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF15 | 1654 | -GTCGCCATTGGCCA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF15 | 1655 | g-CTCCCTCGCCTATA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF15 | 1656 | -GGCCCGGGAGCCGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF7L2 | 1657 | g-ATCCTTTCGGGCGC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF7L2 | 1658 | g-ATGCACACGTCACT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF7L2 | 1659 | -GAACGGAGTAGTCT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF7L2 | 1660 | -GGGATTCTGGGCGA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TCF7L2 | 1661 | g-ATCCTTTCGGGCGC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF7L2 | 1662 | g-ATGCACACGTCACT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF7L2 | 1663 | -GAACGGAGTAGTCT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF7L2 | 1664 | -GGGATTCTGGGCGA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TCF7L2 | 1665 | g-ATCCTTTCGGGCGC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF7L2 | 1666 | g-ATGCACACGTCACT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF7L2 | 1667 | -GAACGGAGTAGTCT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF7L2 | 1668 | -GGGATTCTGGGCGA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TCF7L2 | 1669 | g-ATCCTTTCGGGCGC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF7L2 | 1670 | g-ATGCACACGTCACT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF7L2 | 1671 | -GAACGGAGTAGTCT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF7L2 | 1672 | -GGGATTCTGGGCGA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TCF7L2 | 1673 | g-ATCCTTTCGGGCGC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF7L2 | 1674 | g-ATGCACACGTCACT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF7L2 | 1675 | -GAACGGAGTAGTCT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF7L2 | 1676 | -GGGATTCTGGGCGA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TCF7L2 | 1677 | g-ATCCTTTCGGGCGC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF7L2 | 1678 | g-ATGCACACGTCACT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF7L2 | 1679 | -GAACGGAGTAGTCT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF7L2 | 1680 | -GGGATTCTGGGCGA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TCF7L2 | 1681 | g-ATCCTTTCGGGCGC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF7L2 | 1682 | g-ATGCACACGTCACT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF7L2 | 1683 | -GAACGGAGTAGTCT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF7L2 | 1684 | -GGGATTCTGGGCGA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TCF7L2 | 1685 | g-ATCCTTTCGGGCGC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF7L2 | 1686 | g-ATGCACACGTCACT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF7L2 | 1687 | -GAACGGAGTAGTCT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF7L2 | 1688 | -GGGATTCTGGGCGA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TCF7L2 | 1689 | g-ATCCTTTCGGGCGC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF7L2 | 1690 | g-ATGCACACGTCACT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF7L2 | 1691 | -GAACGGAGTAGTCT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF7L2 | 1692 | -GGGATTCTGGGCGA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TCF7L2 | 1693 | g-ATCCTTTCGGGCGC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF7L2 | 1694 | g-ATGCACACGTCACT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF7L2 | 1695 | -GAACGGAGTAGTCT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF7L2 | 1696 | -GGGATTCTGGGCGA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TCF7L2 | 1697 | g-ATCCTTTCGGGCGC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF7L2 | 1698 | g-ATGCACACGTCACT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF7L2 | 1699 | -GAACGGAGTAGTCT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF7L2 | 1700 | -GGGATTCTGGGCGA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TCF7L2 | 1701 | g-ATCCTTTCGGGCGC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF7L2 | 1702 | g-ATGCACACGTCACT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF7L2 | 1703 | -GAACGGAGTAGTCT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF7L2 | 1704 | -GGGATTCTGGGCGA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TCF7L2 | 1705 | g-ATCCTTTCGGGCGC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF7L2 | 1706 | g-ATGCACACGTCACT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF7L2 | 1707 | -GAACGGAGTAGTCT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF7L2 | 1708 | -GGGATTCTGGGCGA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TCF7L2 | 1709 | g-ATCCTTTCGGGCGC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF7L2 | 1710 | g-ATGCACACGTCACT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF7L2 | 1711 | -GAACGGAGTAGTCT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF7L2 | 1712 | -GGGATTCTGGGCGA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TCF7L2 | 1713 | g-ATCCTTTCGGGCGC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF7L2 | 1714 | g-ATGCACACGTCACT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF7L2 | 1715 | -GAACGGAGTAGTCT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF7L2 | 1716 | -GGGATTCTGGGCGA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TCF7L2 | 1717 | g-ATCCTTTCGGGCGC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF7L2 | 1718 | g-ATGCACACGTCACT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF7L2 | 1719 | -GAACGGAGTAGTCT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF7L2 | 1720 | -GGGATTCTGGGCGA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TCF7L2 | 1721 | g-ATCCTTTCGGGCGC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF7L2 | 1722 | g-ATGCACACGTCACT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF7L2 | 1723 | -GAACGGAGTAGTCT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF7L2 | 1724 | -GGGATTCTGGGCGA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TCF7L2 | 1725 | g-ATCCTTTCGGGCGC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF7L2 | 1726 | g-ATGCACACGTCACT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF7L2 | 1727 | -GAACGGAGTAGTCT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF7L2 | 1728 | -GGGATTCTGGGCGA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TCF7L2 | 1729 | g-ATCCTTTCGGGCGC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF7L2 | 1730 | g-ATGCACACGTCACT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF7L2 | 1731 | -GAACGGAGTAGTCT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF7L2 | 1732 | -GGGATTCTGGGCGA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TCF7L2 | 1733 | g-ATCCTTTCGGGCGC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF7L2 | 1734 | g-ATGCACACGTCACT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF7L2 | 1735 | -GAACGGAGTAGTCT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF7L2 | 1736 | -GGGATTCTGGGCGA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TCF7L2 | 1737 | g-ATCCTTTCGGGCGC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF7L2 | 1738 | g-ATGCACACGTCACT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF7L2 | 1739 | -GAACGGAGTAGTCT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF7L2 | 1740 | -GGGATTCTGGGCGA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TCF7L2 | 1741 | g-ATCCTTTCGGGCGC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF7L2 | 1742 | g-ATGCACACGTCACT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF7L2 | 1743 | -GAACGGAGTAGTCT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF7L2 | 1744 | -GGGATTCTGGGCGA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TCF7L2 | 1745 | g-ATCCTTTCGGGCGC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF7L2 | 1746 | g-ATGCACACGTCACT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF7L2 | 1747 | -GAACGGAGTAGTCT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TCF7L2 | 1748 | -GGGATTCTGGGCGA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TWIST1 | 1749 | g-ACCCCGAGGAAGGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TWIST1 | 1750 | -GCGCTAGGGTTCGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TWIST1 | 1751 | g-AGCCCCATCCGGAG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TWIST1 | 1752 | -GGCCTGACGTGAGG-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| TWIST1 | 1753 | g-ACCCCGAGGAAGGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TWIST1 | 1754 | -GCGCTAGGGTTCGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TWIST1 | 1755 | g-AGCCCCATCCGGAG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TWIST1 | 1756 | -GGCCTGACGTGAGG-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| TWIST1 | 1757 | g-ACCCCGAGGAAGGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TWIST1 | 1758 | -GCGCTAGGGTTCGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TWIST1 | 1759 | g-AGCCCCATCCGGAG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TWIST1 | 1760 | -GGCCTGACGTGAGG-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| TWIST1 | 1761 | g-ACCCCGAGGAAGGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TWIST1 | 1762 | -GCGCTAGGGTTCGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TWIST1 | 1763 | g-AGCCCCATCCGGAG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TWIST1 | 1764 | -GGCCTGACGTGAGG-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| TWIST1 | 1765 | g-ACCCCGAGGAAGGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TWIST1 | 1766 | -GCGCTAGGGTTCGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TWIST1 | 1767 | g-AGCCCCATCCGGAG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TWIST1 | 1768 | -GGCCTGACGTGAGG-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| TWIST1 | 1769 | g-ACCCCGAGGAAGGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TWIST1 | 1770 | -GCGCTAGGGTTCGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TWIST1 | 1771 | g-AGCCCCATCCGGAG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TWIST1 | 1772 | -GGCCTGACGTGAGG-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| TWIST1 | 1773 | g-ACCCCGAGGAAGGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TWIST1 | 1774 | -GCGCTAGGGTTCGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TWIST1 | 1775 | g-AGCCCCATCCGGAG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TWIST1 | 1776 | -GGCCTGACGTGAGG-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| TWIST1 | 1777 | g-ACCCCGAGGAAGGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TWIST1 | 1778 | -GCGCTAGGGTTCGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TWIST1 | 1779 | g-AGCCCCATCCGGAG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TWIST1 | 1780 | -GGCCTGACGTGAGG-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| TWIST1 | 1781 | g-ACCCCGAGGAAGGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TWIST1 | 1782 | -GCGCTAGGGTTCGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TWIST1 | 1783 | g-AGCCCCATCCGGAG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TWIST1 | 1784 | -GGCCTGACGTGAGG-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| TWIST1 | 1785 | g-ACCCCGAGGAAGGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TWIST1 | 1786 | -GCGCTAGGGTTCGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TWIST1 | 1787 | g-AGCCCCATCCGGAG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TWIST1 | 1788 | -GGCCTGACGTGAGG-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| TWIST1 | 1789 | g-ACCCCGAGGAAGGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TWIST1 | 1790 | -GCGCTAGGGTTCGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TWIST1 | 1791 | g-AGCCCCATCCGGAG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TWIST1 | 1792 | -GGCCTGACGTGAGG-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| TWIST1 | 1793 | g-ACCCCGAGGAAGGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TWIST1 | 1794 | -GCGCTAGGGTTCGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TWIST1 | 1795 | g-AGCCCCATCCGGAG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TWIST1 | 1796 | -GGCCTGACGTGAGG-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| TWIST1 | 1797 | g-ACCCCGAGGAAGGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TWIST1 | 1798 | -GCGCTAGGGTTCGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TWIST1 | 1799 | g-AGCCCCATCCGGAG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TWIST1 | 1800 | -GGCCTGACGTGAGG-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| TWIST1 | 1801 | g-ACCCCGAGGAAGGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TWIST1 | 1802 | -GCGCTAGGGTTCGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TWIST1 | 1803 | g-AGCCCCATCCGGAG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TWIST1 | 1804 | -GGCCTGACGTGAGG-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| TWIST1 | 1805 | g-ACCCCGAGGAAGGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TWIST1 | 1806 | -GCGCTAGGGTTCGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TWIST1 | 1807 | g-AGCCCCATCCGGAG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TWIST1 | 1808 | -GGCCTGACGTGAGG-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| TWIST1 | 1809 | g-ACCCCGAGGAAGGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TWIST1 | 1810 | -GCGCTAGGGTTCGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TWIST1 | 1811 | g-AGCCCCATCCGGAG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TWIST1 | 1812 | -GGCCTGACGTGAGG-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| TWIST1 | 1813 | g-ACCCCGAGGAAGGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TWIST1 | 1814 | -GCGCTAGGGTTCGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TWIST1 | 1815 | g-AGCCCCATCCGGAG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TWIST1 | 1816 | -GGCCTGACGTGAGG-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| TWIST1 | 1817 | g-ACCCCGAGGAAGGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TWIST1 | 1818 | -GCGCTAGGGTTCGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TWIST1 | 1819 | g-AGCCCCATCCGGAG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TWIST1 | 1820 | -GGCCTGACGTGAGG-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| TWIST1 | 1821 | g-ACCCCGAGGAAGGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TWIST1 | 1822 | -GCGCTAGGGTTCGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TWIST1 | 1823 | g-AGCCCCATCCGGAG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TWIST1 | 1824 | -GGCCTGACGTGAGG-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| TWIST1 | 1825 | g-ACCCCGAGGAAGGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TWIST1 | 1826 | -GCGCTAGGGTTCGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TWIST1 | 1827 | g-AGCCCCATCCGGAG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TWIST1 | 1828 | -GGCCTGACGTGAGG-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| TWIST1 | 1829 | g-ACCCCGAGGAAGGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TWIST1 | 1830 | -GCGCTAGGGTTCGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TWIST1 | 1831 | g-AGCCCCATCCGGAG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TWIST1 | 1832 | -GGCCTGACGTGAGG-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| TWIST1 | 1833 | g-ACCCCGAGGAAGGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TWIST1 | 1834 | -GCGCTAGGGTTCGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TWIST1 | 1835 | g-AGCCCCATCCGGAG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TWIST1 | 1836 | -GGCCTGACGTGAGG-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| TWIST1 | 1837 | g-ACCCCGAGGAAGGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TWIST1 | 1838 | -GCGCTAGGGTTCGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TWIST1 | 1839 | g-AGCCCCATCCGGAG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| TWIST1 | 1840 | -GGCCTGACGTGAGG-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| UNCX | 1841 | -GCCCCGAGTGAAGT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| UNCX | 1842 | g-CGGCCCGGGGAATT-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| UNCX | 1843 | g-ATTAGCAATCGATA-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| UNCX | 1844 | g-TGTCACAACAAGAC-GTTTTAGAGCTA- |
| | | CCGTCGTTGTACCGTCTACTTGAC- |
| | | TTTTTTTTcagccaactccaaTTTTTTTT- |
| | | GTCAAGTAGACGGTACAACGACGG |
| UNCX | 1845 | -GCCCCGAGTGAAGT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| UNCX | 1846 | g-CGGCCCGGGGAATT-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| UNCX | 1847 | g-ATTAGCAATCGATA-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| UNCX | 1848 | g-TGTCACAACAAGAC-GTTTTAGAGCTA- |
| | | CCTTCCCGACTTCCGTAGGAGCGC- |
| | | TTTTTTTTcagccaactccaaTTTTTTTT- |
| | | GCGCTCCTACGGAAGTCGGGAAGG |
| UNCX | 1849 | -GCCCCGAGTGAAGT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| UNCX | 1850 | g-CGGCCCGGGGAATT-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| UNCX | 1851 | g-ATTAGCAATCGATA-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| UNCX | 1852 | g-TGTCACAACAAGAC-GTTTTAGAGCTA- |
| | | CCTGAAGGTAAAGATCGGGTCCTC- |
| | | TTTTTTTTcagccaactccaaTTTTTTTT- |
| | | GAGGACCCGATCTTTACCTTCAGG |
| UNCX | 1853 | -GCCCCGAGTGAAGT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| UNCX | 1854 | g-CGGCCCGGGGAATT-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| UNCX | 1855 | g-ATTAGCAATCGATA-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| UNCX | 1856 | g-TGTCACAACAAGAC-GTTTTAGAGCTA- |
| | | CCTGGTTATAGCGGACGCACTGT- |
| | | TTTTTTTTcagccaactccaaTTTTTTTT- |
| | | ACAGTGCGTCCGCTATAACCAGG |
| UNCX | 1857 | -GCCCCGAGTGAAGT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| UNCX | 1858 | g-CGGCCCGGGGAATT-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| UNCX | 1859 | g-ATTAGCAATCGATA-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| UNCX | 1860 | g-TGTCACAACAAGAC-GTTTTAGAGCTA- |
| | | CCGCATCGTATTATCTGCGAAGC- |
| | | TTTTTTTTcagccaactccaaTTTTTTTT- |
| | | GCTTCGCAGATAATACGATGCGG |
| UNCX | 1861 | -GCCCCGAGTGAAGT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| UNCX | 1862 | g-CGGCCCGGGGAATT-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| UNCX | 1863 | g-ATTAGCAATCGATA-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| UNCX | 1864 | g-TGTCACAACAAGAC-GTTTTAGAGCTA- |
| | | CCGCTAGGACGTTCAACGGAGTC- |
| | | TTTTTTTTcagccaactccaaTTTTTTTT- |
| | | GACTCCGTTGAACGTCCTAGCGG |
| UNCX | 1865 | -GCCCCGAGTGAAGT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| UNCX | 1866 | g-CGGCCCGGGGAATT-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| UNCX | 1867 | g-ATTAGCAATCGATA-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| UNCX | 1868 | g-TGTCACAACAAGAC-GTTTTAGAGCTA- |
| | | CCATAGCGGTACTATCGGCATCT- |
| | | TTTTTTTTcagccaactccaaTTTTTTTT- |
| | | AGATGCCGATAGTACCGCTATGG |
| UNCX | 1869 | -GCCCCGAGTGAAGT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| UNCX | 1870 | g-CGGCCCGGGGAATT-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| UNCX | 1871 | g-ATTAGCAATCGATA-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| UNCX | 1872 | g-TGTCACAACAAGAC-GTTTTAGAGCTA- |
| | | CCTCGTACTAGTCTCGCATGACC- |
| | | TTTTTTTTcagccaactccaaTTTTTTTT- |
| | | GGTCATGCGAGACTAGTACGAGG |
| UNCX | 1873 | -GCCCCGAGTGAAGT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| UNCX | 1874 | g-CGGCCCGGGGAATT-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| UNCX | 1875 | g-ATTAGCAATCGATA-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| UNCX | 1876 | g-TGTCACAACAAGAC-GTTTTAGAGCTA- |
| | | CCGATAGTGCTAAACGACGCTTT- |
| | | TTTTTTTTcagccaactccaaTTTTTTTT- |
| | | AAAGCGTCGTTTAGCACTATCGG |
| UNCX | 1877 | -GCCCCGAGTGAAGT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| UNCX | 1878 | g-CGGCCCGGGGAATT-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| UNCX | 1879 | g-ATTAGCAATCGATA-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| UNCX | 1880 | g-TGTCACAACAAGAC-GTTTTAGAGCTA- |
| | | CCCGTTATCGACGTTCAAGGAAC- |
| | | TTTTTTTTcagccaactccaaTTTTTTTT- |
| | | GTTCCTTGAACGTCGATAACGGG |
| UNCX | 1881 | -GCCCCGAGTGAAGT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| UNCX | 1882 | g-CGGCCCGGGGAATT-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| UNCX | 1883 | g-ATTAGCAATCGATA-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| UNCX | 1884 | g-TGTCACAACAAGAC-GTTTTAGAGCTA- |
| | | CCGATCAATGCGGTCTGCGTAAC- |
| | | TTTTTTTTcagccaactccaaTTTTTTTT- |
| | | GTTACGCAGACCGCATTGATCGG |
| UNCX | 1885 | -GCCCCGAGTGAAGT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| UNCX | 1886 | g-CGGCCCGGGGAATT-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| UNCX | 1887 | g-ATTAGCAATCGATA-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| UNCX | 1888 | g-TGTCACAACAAGAC-GTTTTAGAGCTA- |
| | | CCCTTACTCGCGATACTCTCGAC- |
| | | TTTTTTTTcagccaactccaaTTTTTTTT- |
| | | GTCGAGAGTATCGCGAGTAAGGG |
| UNCX | 1889 | -GCCCCGAGTGAAGT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| UNCX | 1890 | g-CGGCCCGGGGAATT-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| UNCX | 1891 | g-ATTAGCAATCGATA-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| UNCX | 1892 | g-TGTCACAACAAGAC-GTTTTAGAGCTA- |
| | | CCGCGGACGGTAAGCACGGAATA- |
| | | TTTTTTTTcagccaactccaaTTTTTTTT- |
| | | TATTCCGTGCTTACCGTCCGCGG |
| UNCX | 1893 | -GCCCCGAGTGAAGT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| UNCX | 1894 | g-CGGCCCGGGGAATT-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| UNCX | 1895 | g-ATTAGCAATCGATA-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| UNCX | 1896 | g-TGTCACAACAAGAC-GTTTTAGAGCTA- |
| | | CCTATAGTCACGAATCGTAGGGT- |
| | | TTTTTTTTcagccaactccaaTTTTTTTT- |
| | | ACCCTACGATTCGTGACTATAGG |
| UNCX | 1897 | -GCCCCGAGTGAAGT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| UNCX | 1898 | g-CGGCCCGGGGAATT-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| UNCX | 1899 | g-ATTAGCAATCGATA-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| UNCX | 1900 | g-TGTCACAACAAGAC-GTTTTAGAGCTA- |
| | | CCCGTCGTCACGTATGCGTAGCA- |
| | | TTTTTTTTcagccaactccaaTTTTTTTT- |
| | | TGCTACGCATACGTGACGACGGG |
| UNCX | 1901 | -GCCCCGAGTGAAGT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| UNCX | 1902 | g-CGGCCCGGGGAATT-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| UNCX | 1903 | g-ATTAGCAATCGATA-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| UNCX | 1904 | g-TGTCACAACAAGAC-GTTTTAGAGCTA- |
| | | CCCGAGAGGTAGTAGACGACGCA- |
| | | TTTTTTTTcagccaactccaaTTTTTTTT- |
| | | TGCGTCGTCTACTACCTCTCGGG |
| UNCX | 1905 | -GCCCCGAGTGAAGT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| UNCX | 1906 | g-CGGCCCGGGGAATT-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| UNCX | 1907 | g-ATTAGCAATCGATA-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| UNCX | 1908 | g-TGTCACAACAAGAC-GTTTTAGAGCTA- |
| | | CCGACGTCTACGGACGGGAGCTA- |
| | | TTTTTTTTcagccaactccaaTTTTTTTT- |
| | | TAGCTCCCGTCCGTAGACGTCGG |
| UNCX | 1909 | -GCCCCGAGTGAAGT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| UNCX | 1910 | g-CGGCCCGGGGAATT-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| UNCX | 1911 | g-ATTAGCAATCGATA-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| UNCX | 1912 | g-TGTCACAACAAGAC-GTTTTAGAGCTA- |
| | | CCAAAGCTCATTCGCGACTGAGC- |
| | | TTTTTTTTcagccaactccaaTTTTTTTT- |
| | | GCTCAGTCGCGAATGAGCTTTGG |
| UNCX | 1913 | -GCCCCGAGTGAAGT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| UNCX | 1914 | g-CGGCCCGGGGAATT-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| UNCX | 1915 | g-ATTAGCAATCGATA-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| UNCX | 1916 | g-TGTCACAACAAGAC-GTTTTAGAGCTA- |
| | | CCATGGCACGATCGACACGGAGG- |
| | | TTTTTTTTcagccaactccaaTTTTTTTT- |
| | | CCTCCGTGTCGATCGTGCCATGG |
| UNCX | 1917 | -GCCCCGAGTGAAGT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| UNCX | 1918 | g-CGGCCCGGGGAATT-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| UNCX | 1919 | g-ATTAGCAATCGATA-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| UNCX | 1920 | g-TGTCACAACAAGAC-GTTTTAGAGCTA- |
| | | CCCGAACTCTCTATCGAACGAGT- |
| | | TTTTTTTTcagccaactccaaTTTTTTTT- |
| | | ACTCGTTCGATAGAGAGTTCGGG |
| UNCX | 1921 | -GCCCCGAGTGAAGT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| UNCX | 1922 | g-CGGCCCGGGGAATT-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| UNCX | 1923 | g-ATTAGCAATCGATA-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| UNCX | 1924 | g-TGTCACAACAAGAC-GTTTTAGAGCTA- |
| | | CCTACACGATAGTGGTCGTGCGC- |
| | | TTTTTTTTcagccaactccaaTTTTTTTT- |
| | | GCGCACGACCACTATCGTGTAGG |
| UNCX | 1925 | -GCCCCGAGTGAAGT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| UNCX | 1926 | g-CGGCCCGGGGAATT-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| UNCX | 1927 | g-ATTAGCAATCGATA-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| UNCX | 1928 | g-TGTCACAACAAGAC-GTTTTAGAGCTA- |
| | | CCCTGGCGGAACGTTCGAGTAAT- |
| | | TTTTTTTTcagccaactccaaTTTTTTTT- |
| | | ATTACTCGAACGTTCCGCCAGGG |
| UNCX | 1929 | -GCCCCGAGTGAAGT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| UNCX | 1930 | g-CGGCCCGGGGAATT-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| UNCX | 1931 | g-ATTAGCAATCGATA-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |
| UNCX | 1932 | g-TGTCACAACAAGAC-GTTTTAGAGCTA- |
| | | CCTACACTCGACATCGGTAGCTA- |
| | | TTTTTTTTcagccaactccaaTTTTTTTT- |
| | | TAGCTACCGATGTCGAGTGTAGG |

TABLE 2
Sequences of spacers and target gene

| | | SEQ ID |
|---|---|---|
| Gene target | spacer | NO: |
| ACAN | TGTGACACATACAT | 1940 |
| ACAN | GGGCGGCCGACAGG | 1941 |
| ACAN | GTGCCCGGACTCTG | 1942 |
| ACAN | CCCTGGCTCCGGAC | 1943 |
| COL2A1 | CGCCAGCCTCGAAA | 1944 |
| COL2A1 | GAACCGCCCGCCCC | 1945 |
| COL2A1 | CGGCGCACTAGGGG | 1946 |
| COL2A1 | GCCCCGGGTTTGGG | 1947 |
| MEF2D | CCGCAGCCTAGCTT | 1948 |
| MEF2D | TGACGGACAGGCGT | 1949 |
| MEF2D | CGGGGCCGCGTCCT | 1950 |
| MEF2D | CAGAGGCCTTTAAA | 1951 |
| pax9 | GCGTGGGGTAAGGG | 1952 |
| pax9 | CACGGCAATTTCTC | 1953 |
| pax9 | GTGACGCTAATATG | 1954 |
| pax9 | CCTAGGGGTAGGGA | 1955 |
| pax9 (set2) | AGGGGCGGGTCCGA | 1956 |
| pax9 (set2) | CTAAGCAGCAACGT | 1957 |
| pax9 (set2) | GCTAGCTCCCCACC | 1958 |
| pax9 (set2) | AGCTGCATGGTGAT | 1959 |
| RUNX2 | CCTTACAGGAGTTT | 1960 |
| RUNX2 | CACTATTACTGGAG | 1961 |
| RUNX2 | ACTGCCTACCACTG | 1962 |
| RUNX2 | TGTATCACATTCTG | 1963 |
| RUNX2 (set2) | AAGAGGTAAGTCGT | 1964 |
| RUNX2 (set2) | gcgcgcggcaatgc | 1965 |
| RUNX2 (set2) | AAGGAGCCTAGCCG | 1966 |
| RUNX2 (set2) | ACGCGGTGCCCCAA | 1967 |
| SOX5 | TAACCACACAATGA | 1968 |
| SOX5 | ACTCTCTTGTATTC | 1969 |
| SOX5 | AACAATGGCCAAGC | 1970 |
| SOX5 | TTCTGTCTTCAATA | 1971 |
| SOX5 (set2) | CTCTAGACAACCTC | 1972 |
| SOX5 (set2) | GACAAGTTGTTTAC | 1973 |
| SOX5 (set2) | AGAGAGCCTAGAAA | 1974 |
| SOX5 (set2) | GGTCAAGGAGTCCT | 1975 |
| SOX5 (set3) | TTGAGAGCAACACG | 1976 |
| SOX5 (set3) | TCATGACCAGCAAA | 1977 |
| SOX5 (set3) | CGGTCACCGCGGCC | 1978 |
| SOX5 (set3) | GTGCAAATAACAAA | 1979 |
| SOX6 | CCAAATGCAAGGAT | 1980 |
| SOX6 | CCAATCCTTGCATT | 1981 |
| SOX6 | GTGATAGTTAGCAA | 1982 |
| SOX6 | TGAAAAAGAACTCC | 1983 |
| SOX6 (set2) | ATAAGCTCAAAAGC | 1984 |
| SOX6 (set2) | AGTTGGAGTTACTA | 1985 |
| SOX6 (set2) | TTCAAGGACATGAA | 1986 |
| SOX6 (set2) | AACTACACTCAGTT | 1987 |
| SOX6 (set3) | GGGTTCCACGCCTT | 1988 |
| SOX6 (set3) | GTTTGTTCTACCTA | 1989 |
| SOX6 (set3) | GAGCAAAACAATTT | 1990 |
| SOX6 (set3) | TAAATGTGCCTGAC | 1991 |
| SOX6 (set4) | TGATGTTGCACGAC | 1992 |
| SOX6 (set4) | TGAAGCAACTACTA | 1993 |
| SOX6 (set4) | ATCGGCGAGACCAG | 1994 |
| SOX6 (set4) | TGAGACAAGAGGCG | 1995 |
| SOX8 | CTCAGGCGCGAGAC | 1996 |
| SOX8 | GCTTTCTTTATGGG | 1997 |
| SOX8 | AGGTCGCCGCGCGG | 1998 |
| SOX8 | gcccggcTACCCAT | 1999 |
| SOX9 | CAAACTTACACACT | 2000 |
| SOX9 | AAAGGGCGGACGGT | 2001 |
| SOX9 | ATTGGACCCGATTT | 2002 |
| SOX9 | GTGGCTCTAAGGTG | 2003 |
| SP1 | TGAGACGTAGGGAT | 2004 |
| SP1 | GCGAGTCTTGCCAT | 2005 |
| SP1 | GGACCGGACAGGGA | 2006 |
| SP1 | CCGCCCAATGAGGG | 2007 |
| TCF15 | AAAAGTTCGGATCT | 2008 |
| TCF15 | GTCGCCATTGGCCA | 2009 |
| TCF15 | CTCCCTCGCCTATA | 2010 |
| TCF15 | GGCCCGGGAGCCGG | 2011 |
| TCF7L2 | ATCCTTTCGGGCGC | 2012 |
| TCF7L2 | ATGCACACGTCACT | 2013 |
| TCF7L2 | GAACGGAGTAGTCT | 2014 |
| TCF7L2 | GGGATTCTGGGCGA | 2015 |
| TWIST1 | ACCCCGAGGAAGGG | 2016 |
| TWIST1 | GCGCTAGGGTTCGG | 2017 |
| TWIST1 | AGCCCCATCCGGAG | 2018 |
| TWIST1 | GGCCTGACGTGAGG | 2019 |
| UNCX | GCCCCGAGTGAAGT | 2020 |
| UNCX | CGGCCCGGGGAATT | 2021 |
| UNCX | ATTAGCAATCGATA | 2022 |
| UNCX | TGTCACAACAAGAC | 2023 |

TABLE 3
Sequences of Stem1 and Stem2

| SEQ ID | | SEQ ID | |
|---|---|---|---|
| NO: | stem1 | NO: | stem2 (reverse complement) |
| 2024 | CCTACACTCGACATCGGTAGC | 2047 | TAGCTACCGATGTCGAGTGTA |
| | TA | | GG |
| 2025 | CCCTGGCGGAACGTTCGAGTA | 2048 | ATTACTCGAACGTTCCGCCAGG |
| | AT | | G |
| 2026 | CCTACACGATAGTGGTCGTGC | 2049 | GCGCACGACCACTATCGTGTA |
| | GC | | GG |
| 2027 | CCCGAACTCTCTATCGAACGA | 2050 | ACTCGTTCGATAGAGAGTTCG |
| | GT | | GG |
| 2028 | CCATGGCACGATCGACACGGA | 2051 | CCTCCGTGTCGATCGTGCCATG |
| | GG | | G |
| 2029 | CCAAAGCTCATTCGCGACTGA | 2052 | GCTCAGTCGCGAATGAGCTTTG |
| | GC | | G |
| 2030 | CCGACGTCTACGGACGGGAGC | 2053 | TAGCTCCCGTCCGTAGACGTCG |
| | TA | | G |
| 2031 | CCCGAGAGGTAGTAGACGACG | 2054 | TGCGTCGTCTACTACCTCTCGG |
| | CA | | G |
| 2032 | CCCGTCGTCACGTATGCGTAG | 2055 | TGCTACGCATACGTGACGACG |
| | CA | | GG |
| 2033 | CCTATAGTCACGAATCGTAGG | 2056 | ACCCTACGATTCGTGACTATAG |
| | GT | | G |
| 2034 | CCGCGGACGGTAAGCACGGAA | 2057 | TATTCCGTGCTTACCGTCCGCG |
| | TA | | G |
| 2035 | CCCTTACTCGCGATACTCTCGA | 2058 | GTCGAGAGTATCGCGAGTAAG |
| | C | | GG |
| 2036 | CCGATCAATGCGGTCTGCGTA | 2059 | GTTACGCAGACCGCATTGATC |
| | AC | | GG |
| 2037 | CCCGTTATCGACGTTCAAGGA | 2060 | GTTCCTTGAACGTCGATAACGG |
| | AC | | G |
| 2038 | CCGATAGTGCTAAACGACGCT | 2061 | AAAGCGTCGTTTAGCACTATCG |
| | TT | | G |
| 2039 | CCTCGTACTAGTCTCGCATGA | 2062 | GGTCATGCGAGACTAGTACGA |
| | CC | | GG |
| 2040 | CCATAGCGGTACTATCGGCAT | 2063 | AGATGCCGATAGTACCGCTAT |
| | CT | | GG |
| 2041 | CCGCTAGGACGTTCAACGGAG | 2064 | GACTCCGTTGAACGTCCTAGCG |
| | TC | | G |
| 2042 | CCGCATCGTATTATCTGCGAA | 2065 | GCTTCGCAGATAATACGATGC |
| | GC | | GG |
| 2043 | CCTGGTTATAGCGGACGCACT | 2066 | ACAGTGCGTCCGCTATAACCA |
| | GT | | GG |
| 2044 | CCTGAAGGTAAAGATCGGGTC | 2067 | GAGGACCCGATCTTTACCTTCA |
| | CTC | | GG |
| 2045 | CCTTCCCGACTTCCGTAGGAG | 2068 | GCGCTCCTACGGAAGTCGGGA |
| | CGC | | AGG |
| 2046 | CCGTCGTTGTACCGTCTACTTG | 2069 | GTCAAGTAGACGGTACAACGA |
| | AC | | CGG |

### EMBODIMENTS

The following non-limiting embodiments provide illustrative examples of the invention, but do not limit the scope of the invention.
1. A method for conversion of a plurality of stem cells into a plurality of chondrogenic cells, via modulation of expression levels of a plurality of distinct target genes comprising a first homeobox protein and a second homeobox protein, the method comprising:
a) contacting a first polynucleotide sequence in the plurality of stem cells by a first heterologous gene regulating moiety to modulate expression level of the first homeobox protein that is operatively coupled to the first polynucleotide sequence; and
b) contacting a second polynucleotide sequence in the plurality of stem cells by a second heterologous gene regulating moiety, to modulate expression level of the second homeobox protein that is operatively coupled to the second polynucleotide sequence.
2. The method of embodiment 1, wherein (b) is performed subsequent to (a), and wherein the steps of (a) and (b) effect modulation of the first homeobox protein and the second homeobox protein in a sequential manner.
3. The method of embodiment 1, wherein (i) the first polynucleotide sequence is upstream to or encodes the first homeobox protein or (ii) the second polynucleotide sequence is upstream to or encodes the second homeobox protein.
4. The method of embodiment 1, wherein (iii) the expression level of the first homeobox protein is enhanced upon the contacting by the first heterologous gene regulating moiety or (iv) the expression level of the second homeobox protein is enhanced upon the contacting by the second heterologous gene regulating moiety.
5. The method of embodiment 1, further comprising modulating expression of an additional target gene of the plurality of target genes, wherein the additional target gene comprises one or more members selected from the group consisting of a T-box transcription factor (TBX), a basic helix-loop-helix transcription factor (bHLH), a Sox, and a collagen.
6. The method of embodiment 5, wherein the expression level of the additional target gene is modulated (i) prior to, (ii) simultaneously with, or (iii) subsequent to the second homeobox protein.
7. The method of embodiment 5, wherein the expression level of the additional target gene is enhanced.
8. The method of embodiment 1, wherein the method comprises contacting the plurality of stem cells with a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
(i) a first gate unit that is preconfigured to effect the first heterologous gene regulating moiety to modulate the expression level of the first homeobox protein; and
(ii) a second gate unit that is preconfigured to effect the second heterologous gene regulating moiety to modulate the expression level of the second homeobox protein, wherein, upon activation of the heterologous genetic circuit, the plurality of gate units operates to effect the conversion.
9. The method of embodiment 8, wherein (i) the first gate unit is activatable to express the first heterologous gene regulating moiety or (ii) the second gate unit is activatable to express the second heterologous gene regulating moiety.
10. The method of embodiment 9, wherein the plurality of gate units comprises an additional gate unit that is preconfigured to modulate expression level of an additional target gene of the plurality of distinct target genes, wherein the additional target gene encodes one or more members selected from the group consisting of a T-box transcription factor (TBX), a basic helix-loop-helix transcription factor (bHLH), a Sox, and a collagen.
11. The method of embodiment 1, wherein the conversion occurs in less than about 14 days, less than about 10 days, less than about 7 days, or less than about 5 days.
12. A method for conversion of a plurality of stem cells into a plurality of chondrogenic cells, via modulation of expression levels of a plurality of distinct target genes comprising a first distinct target gene and a second distinct target gene, the method comprising:
a) contacting a first polynucleotide sequence in the plurality of stem cells by a first heterologous gene regulating moiety to modulate expression level of the first distinct target gene that is operatively coupled to the first polynucleotide sequence; and
b) contacting a second polynucleotide sequence in the plurality of stem cells by a second heterologous gene regulating moiety, to modulate expression level of the second distinct target gene that is operatively coupled to the second polynucleotide sequence, wherein a combination of the first and second distinct target genes is:
i) a first homeobox protein and a second homeobox protein that are different;
ii) two different members selected from the group consisting of a homeobox protein, a T-box transcription factor (TBX), and a basic helix-loop-helix transcription factor (bHLH); or
iii) a first member selected from the group consisting of the homeobox protein, the TBX, and the bHLH, and a second member comprising SOX or collagen.
13. The method of embodiment 12, wherein the combination is (i) the first homeobox protein and the second homeobox protein.
14. The method of embodiment 12, wherein the combination is (ii) the two different members selected from the group consisting of the homeobox protein, the TBX, and the bHLH.
15. The method of embodiment 12, wherein the combination is (iii) the first member selected from the group consisting of the homeobox protein, the TBX, and the bHLH, and the second member comprising the SOX or the collagen.
16. The method of embodiment 12, wherein (b) is performed subsequent to (a), thereby to effect modulation of the first distinct target gene and the second distinct target gene in a sequential manner.
17. The method of embodiment 12, wherein (i) the first polynucleotide sequence is upstream to or encodes the first distinct target gene or (ii) the second polynucleotide sequence is upstream to or encodes the second distinct target gene.
18. The method of embodiment 12, wherein (ii) the expression level of the first distinct target gene is enhanced upon the contacting by the first heterologous gene regulating moiety or (ii) the expression level of the second distinct target gene is enhanced upon the contacting by the second heterologous gene regulating moiety.
19. The method of embodiment 12, wherein the method comprises contacting the plurality of stem cells with a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
   (i) a first gate unit that is preconfigured to effect the first heterologous gene regulating moiety to modulate the expression level of the first distinct target gene; and
   (ii) a second gate unit that is preconfigured to effect the second heterologous gene regulating moiety to modulate the expression level of the second distinct target gene, wherein, upon activation of the heterologous genetic circuit, the plurality of gate units operates to effect the conversion.
20. The method of embodiment 12, wherein the conversion occurs in less than about 14 days, less than about 10 days, less than about 7 days, or less than about 5 days.
21. A method for conversion of a plurality of stem cells towards chondrogenic differentiation, the method comprising:
   contacting a polynucleotide sequence in the plurality of stem cells by a heterologous gene regulating moiety to modulate expression level of a target gene that is operatively coupled to the polynucleotide sequence,
   wherein, within less than 7 days following the contacting, a conversion rate from the plurality of stem cells to chondrogenic cells is characterized to be at least about 30%.
22. The method of embodiment 21, wherein the conversion rate is at least about 40%, at least about 50%, at least about 60%, at least about 70%, or at least about 80%.
23. The method of embodiment 21, wherein the conversion rate is observed within less than about 6 days following the contacting or within less than about 5 days following the contacting.
24. The method of embodiment 21, wherein the expression level of the target gene is enhanced upon the contacting by the heterologous gene regulating moiety.
25. The method of embodiment 21, wherein the target gene comprises one or more members selected from the group consisting of a homeobox protein, a T-box transcription factor (TBX), a basic helix-loop-helix transcription factor (bHLH), a SOX, and a collagen.
26. The method of embodiment 21, wherein the target gene comprises a plurality of distinct target genes comprising a first distinct target and a second distinct target gene, and wherein the contacting comprises:
   (a) contacting a first polynucleotide sequence in the plurality of stem cells by a first heterologous gene regulating moiety to modulate expression level of the first distinct target gene that is operatively coupled to the first polynucleotide sequence; and
   (b) contacting a second polynucleotide sequence in the plurality of stem cells by a second heterologous gene regulating moiety, to modulate expression level of the second distinct target gene that is operatively coupled to the second polynucleotide sequence.
27. The method of embodiment 26, wherein a combination of the first and second distinct target genes is:
(i) a first homeobox protein and a second homeobox protein that are different;
(ii) two different members selected from the group consisting of a homeobox protein, a T-box transcription factor (TBX), and a basic helix-loop-helix transcription factor (bHLH); or
(iii) a first member selected from the group consisting of the homeobox protein, the TBX, and the bHLH, and a second member comprising SOX or collagen.
28. The method of embodiment 26, wherein the steps of (a) and (b) effect modulation of the first distinct target gene and the second distinct target gene in a sequential manner.
29. The method of embodiment 26, wherein the contacting comprises:
   contacting the plurality of stem cells with a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
(i) a first gate unit that is preconfigured to effect the first heterologous gene regulating moiety to modulate the expression level of the first distinct target gene; and
(ii) a second gate unit that is preconfigured to effect the second heterologous gene regulating moiety to modulate the expression level of the second distinct target gene,
   wherein, upon activation of the heterologous genetic circuit, the plurality of gate units operates to effect the conversion.
30. The method of embodiment 21, wherein the target gene is an endogenous target gene.
31. A method for treating a subject in need thereof, the method comprising:
   administering a plurality of chondrogenic cells to the subject, wherein the plurality of chondrogenic cells is prepared by subjecting a plurality of stem cells to ex vivo differentiation,
   wherein, within less than 7 days of the ex vivo differentiation, a conversion rate from the plurality of stem cells to the plurality of chondrogenic cells is characterized to be at least about 30%.
32. The method of embodiment 31, wherein the conversion rate is at least about 40%, at least about 50%, at least about 60%, at least about 70%, or at least about 80%.
33. The method of embodiment 31, wherein the conversion rate is observed within less than about 6 days following the contacting or within less than about 5 days following the contacting.
34. The method of embodiment 31, wherein the plurality of chondrogenic cells is subjected to ex vivo culture for less than about 2 weeks, less than about 10 days, less than about 8 days, less than about 7 days, less than about 6 days, or less than about 5 days.
35. The method of embodiment 31, wherein the ex vivo differentiation comprises modulating expression level of a target gene comprising one or more members selected from the group consisting of a homeobox protein, a T-box transcription factor (TBX), a basic helix-loop-helix transcription factor (bHLH), a SOX, and a collagen.
36. The method of embodiment 35, wherein the target gene is an endogenous target gene.
37. The method of embodiment 35, wherein the target gene comprises a plurality of distinct target genes that comprises:
   (i) a first homeobox protein and a second homeobox protein that are different;
   (ii) two different members selected from the group consisting of a homeobox protein, a T-box transcription factor (TBX), and a basic helix-loop-helix transcription factor (bHLH); or
   (iii) a first member selected from the group consisting of the homeobox protein, the TBX, and the bHLH, and a second member comprising SOX or collagen.
38. The method of any one of the preceding embodiments, wherein the plurality of chondrogenic cells comprises chondroprogenitor cells or chondrocytes.
39. The method of any one of the preceding embodiments, wherein the plurality of chondrogenic cells is characterized to be CD146+/CD73+, CD146+/CD112+, or CD326+/CD309+.
40. The method of any one of the preceding embodiments, wherein the plurality of stem cells comprise pluripotent stem cells (PSCs) or mesodermal cells.
41. The method of any one of the preceding embodiments, wherein the plurality of distinct target genes are endogenous genes of the plurality of stem cells.
42. The method of any one of the preceding embodiments, wherein the first heterologous gene regulating moiety or the second heterologous gene regulating moiety comprises (i) an endonuclease or (ii) a guide nucleic acid (gNA) molecule.
43. The method of embodiment 42, wherein the endonuclease and the gNA form a complex capable of binding to a respective target polynucleotide sequence.
44. The method of embodiment 42, wherein the endonuclease is a Cas protein.
45. The method of any one of the preceding embodiments, wherein the conversion occurs in conditions substantially free of (i) serum and/or (ii) an exogenous cell differentiation regulatory factor.
46. The method of embodiment 45, wherein the exogenous cell differentiation regulatory factor comprises one or more members selected from the group consisting of TGFbeta1, TGFbeta2, TGFbeta3, BMP2, BMP4, BMP6, MP7, and IGF1.
47. The method of embodiment 45, wherein the exogenous cell differentiation regulatory factor is a chondrogenic factor comprising one or more members selected from the group consisting of dexamethasone, ascorbate, insulin, transferrin, and selenous acid.
48. The method of any one of the preceding embodiments, wherein the first homeobox protein, the second homeobox protein, or the homeobox protein is a paired box (PRD)-class homeobox protein.
49. The method of embodiment 48, wherein the PRD-class homeobox protein is MIXL1 or UNCX.
50. The method of any one of the preceding embodiments, wherein the first homeobox protein,
   the second homeobox protein, or the homeobox protein is not a member of Pax3 and Pax7.
51. The method of any one of the preceding embodiments, wherein the TBX comprises one or more members selected from the group consisting of TBXT, TBR1, TBX1, TBX2, TBX3, TBX4, TBX5, TBX6, TBX10, TBX15, TBX18, TBX19, TBX20, TBX21, and TBX22.
52. The method of embodiment 51, wherein the TBX is TBXT or TBX6.
53. The method of any one of the preceding embodiments, wherein the bHLH comprises one or more members selected from the group consisting of Group A bHLH, Group B bHLH, Group C bHLH, Group D bHLH, Group E bHLH, and Group F bHLH.
54. The method of embodiment 53, wherein the bHLH is the Group A bHLH.
55. The method of embodiment 53, wherein the bHLH is MSGN1 or TCF15.
56. The method of any one of the preceding embodiments, wherein the SOX comprises one or more members selected from the group consisting of SOXA, SOXB1, SOXB2, SOXC, SOXD, SOXE, SOXF, SOXG, and SOXH.
57. The method of embodiment 56, wherein the SOX is SOXD.
58. The method of embodiment 57, wherein the SOXD is SOX6.
59. The method of embodiment 56, wherein the SOX is SOXE.
60. The method of embodiment 59, wherein the SOXE is SOX9.
61. The method of any one of the preceding embodiments, wherein the collagen comprises one or more members selected from the group consisting of Type I collagen, Type II collagen, Type III collagen, Type IV collagen, and Type V collagen.
62. The method of embodiment 61, wherein the collagen is Type II collagen.
63. The method of embodiment 62, wherein the Type II collagen is COL2A1.
64. The method of any one of the preceding embodiments, wherein the conversion occurs in less than about 10 days, less than about 7 days, or less than about 5 days.
65. The method of any one of the preceding embodiments, further comprising storing the plurality of tissue-specific progenitor cells or the plurality of chondrogenic cells in a sterile vial.
66. The method of any one of the preceding embodiments, further comprising administering the plurality of tissue-specific progenitor cells or the plurality of chondrogenic cells to a subject in need thereof.
67. The method of any one of the preceding embodiments, wherein the administering comprises one or more knee administrations.
68. A system for conversion of a plurality of stem cells into a plurality of chondrogenic cells, via modulation of expression levels of a plurality of distinct target genes comprising a first homeobox protein and a second homeobox protein, the system comprising:
a) a first heterologous gene regulating moiety configured to bind a first polynucleotide sequence in the plurality of stem cells to modulate expression level of the first homeobox protein that is operatively coupled to the first polynucleotide sequence; and
b) a second heterologous gene regulating moiety configured to bind a second polynucleotide sequence in the plurality of stem cells to modulate expression level of the second homeobox protein that is operatively coupled to the second polynucleotide sequence.
69. The system of embodiment 68, wherein the first and second heterologous gene regulating moieties are configured to effect modulation of the first homeobox protein and the second homeobox protein in a sequential manner.
70. The system of embodiment 68, wherein (i) the first polynucleotide sequence is upstream to or encodes the first homeobox protein or (ii) the second polynucleotide sequence is upstream to or encodes the second homeobox protein.
71. The system of embodiment 68, wherein (ii) the expression level of the first homeobox protein is enhanced upon the contacting by the first heterologous gene regulating moiety or (ii) the expression level of the second homeobox protein is enhanced upon the contacting by the second heterologous gene regulating moiety.
72. The system of embodiment 68, further comprising an additional heterologous gene regulating moiety configured to modulate expression of an additional target gene of the plurality of target genes, wherein the additional target gene comprises one or more members selected from the group consisting of a T-box transcription factor (TBX), a basic helix-loop-helix transcription factor (bHLH), a Sox, and a collagen.
73. The system of embodiment 72, wherein the expression level of the additional target gene is modulated (i) prior to, (ii) simultaneously with, or (iii) subsequent to the second homeobox protein.
74. The system of embodiment 72, wherein the expression level of the additional target gene is enhanced.
75. The system of embodiment 68, comprising a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
   (i) a first gate unit that is preconfigured to effect the first heterologous gene regulating moiety to modulate the expression level of the first homeobox protein; and
   (ii) a second gate unit that is preconfigured to effect the second heterologous gene regulating moiety to modulate the expression level of the second homeobox protein,
   wherein, upon activation of the heterologous genetic circuit, the plurality of gate units operates to effect the conversion.
76. The system of embodiment 75, wherein (i) the first gate unit is activatable to express the first heterologous gene regulating moiety or (ii) the second gate unit is activatable to express the second heterologous gene regulating moiety.
77. The system of embodiment 75, wherein the plurality of gate units comprises an additional gate unit that is preconfigured to modulate expression level of an additional target gene of the plurality of distinct target genes, wherein the additional target gene encodes one or more members selected from the group consisting of a T-box transcription factor (TBX), a basic helix-loop-helix transcription factor (bHLH), a Sox, and a collagen.
78. The system of embodiment 68, wherein the conversion occurs in less than about 14 days, less than about 10 days, less than about 7 days, or less than about 5 days.
79. A system for conversion of a plurality of stem cells into a plurality of chondrogenic cells, via modulation of expression levels of a plurality of distinct target genes comprising a first distinct target gene and a second distinct target gene, the system comprising:
a) a first heterologous gene regulating moiety configured to bind a first polynucleotide sequence in the plurality of stem cells to modulate expression level of the first distinct target gene that is operatively coupled to the first polynucleotide sequence; and
b) a second heterologous gene regulating moiety configured to bind a second polynucleotide sequence in the plurality of stem cells to modulate expression level of the second distinct target gene that is operatively coupled to the second polynucleotide sequence,
   wherein a combination of the first and second distinct target genes is:
      (i) a first homeobox protein and a second homeobox protein that are different;
      (ii) two different members selected from the group consisting of a homeobox protein, a T-box transcription factor (TBX), and a basic helix-loop-helix transcription factor (bHLH); or
      (iii) first member selected from the group consisting of the homeobox protein, the TBX, and the bHLH, and a second member comprising SOX or collagen.
80. The system of embodiment 79, wherein the combination is (i) the first homeobox protein and the second homeobox protein.
81. The system of embodiment 79, wherein the combination is (ii) the two different members selected from the group consisting of the homeobox protein, the TBX, and the bHLH.
82. The system of embodiment 79, wherein the combination is (iii) the first member selected from the group consisting of the homeobox protein, the TBX, and the bHLH, and the second member comprising the SOX or the collagen.
83. The system of embodiment 79, wherein the first and second heterologous gene regulating moieties are configured to effect modulation of the first distinct target gene prior to that of the second distinct target gene in a sequential manner.
84. The system of embodiment 79, wherein (i) the first polynucleotide sequence is upstream to or encodes the first distinct target gene or (ii) the second polynucleotide sequence is upstream to or encodes the second distinct target gene.
85. The system of embodiment 79, wherein (ii) the expression level of the first distinct target gene is enhanced upon the contacting by the first heterologous gene regulating moiety or (ii) the expression level of the second distinct target gene is enhanced upon the contacting by the second heterologous gene regulating moiety.
86. The system of embodiment 79, comprising a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
   (i) a first gate unit that is preconfigured to effect the first heterologous gene regulating moiety to modulate the expression level of the first distinct target gene; and
   (ii) a second gate unit that is preconfigured to effect the second heterologous gene regulating moiety to modulate the expression level of the second distinct target gene,
   wherein, upon activation of the heterologous genetic circuit, the plurality of gate units operates to effect the conversion.
87. The system of embodiment 79, wherein the conversion occurs in less than about 14 days, less than about 10 days, less than about 7 days, or less than about 5 days.
88. A system for conversion of a plurality of stem cells towards chondrogenic differentiation, the system comprising:
   a heterologous gene regulating moiety configured to bind a polynucleotide sequence in the plurality of stem cells to modulate expression level of a target gene that is operatively coupled to the polynucleotide sequence,
   wherein, within less than 7 days following the contacting, a conversion rate from the plurality of stem cells to chondrogenic cells is characterized to be at least about 30%.
89. The system of embodiment 88, wherein the conversion rate is at least about 40%, at least about 50%, at least about 60%, at least about 70%, or at least about 80%.
90. The system of embodiment 88, wherein the conversion rate is observed within less than about 6 days following the contacting or within less than about 5 days following the contacting.
91. The system of embodiment 88, wherein the expression level of the target gene is enhanced upon the contacting by the heterologous gene regulating moiety.
92. The system of embodiment 88, wherein the target gene comprises one or more members selected from the group consisting of a homeobox protein, a T-box transcription factor (TBX), a basic helix-loop-helix transcription factor (bHLH), a SOX, and a collagen.
93. The system of embodiment 88, wherein the target gene comprises a plurality of distinct target genes comprising a first distinct target and a second distinct target gene, and wherein the system comprises:
a) a first heterologous gene regulating moiety configured to bind a first polynucleotide sequence in the plurality of stem cells to modulate expression level of the first distinct target gene that is operatively coupled to the first polynucleotide sequence; and
b) a second heterologous gene regulating moiety configured to bind a second polynucleotide sequence in the plurality of stem cells to modulate expression level of the second distinct target gene that is operatively coupled to the second polynucleotide sequence.
94. The system of embodiment 93, wherein a combination of the first and second distinct target genes is:
   (i) a first homeobox protein and a second homeobox protein that are different;
   (ii) two different members selected from the group consisting of a homeobox protein, a T-box transcription factor (TBX), and a basic helix-loop-helix transcription factor (bHLH); or
   (iii) a first member selected from the group consisting of the homeobox protein, the TBX, and the bHLH, and a second member comprising SOX or collagen.
95. The system of embodiment 93, wherein the first and second heterologous gene regulating moieties are configured to effect modulation of the first distinct target gene and the second distinct target gene in a sequential manner.
96. The system of embodiment 93, comprising a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
   (i) a first gate unit that is preconfigured to effect the first heterologous gene regulating moiety to modulate the expression level of the first distinct target gene; and
   (ii) a second gate unit that is preconfigured to effect the second heterologous gene regulating moiety to modulate the expression level of the second distinct target gene,
   wherein, upon activation of the heterologous genetic circuit, the plurality of gate units operates to effect the conversion.
97. The system of embodiment 88, wherein the target gene is an endogenous target gene.
98. A composition comprising any one of the preceding embodiments.
99. A composition for treating a subject in need thereof, the composition comprising:
   a plurality of chondrogenic cells prepared by subjecting a plurality of stem cells to ex vivo differentiation,
   wherein, within less than 7 days of the ex vivo differentiation, a conversion rate from the plurality of stem cells to the plurality of chondrogenic cells is characterized to be at least about 30%.
100. The composition of embodiment 99, wherein the conversion rate is at least about 40%, at least about 50%, at least about 60%, at least about 70%, or at least about 80%.
101. The composition of embodiment 99, wherein the conversion rate is observed within less than about 6 days following the contacting or within less than about 5 days following the contacting.
102. The composition of embodiment 99, wherein the plurality of chondrogenic cells is subjected to ex vivo culture for less than about 2 weeks, less than about 10 days, less than about 8 days, less than about 7 days, less than about 6 days, or less than about 5 days.
103. The composition of embodiment 99, wherein the ex vivo differentiation is characterized by modulation of expression level of a target gene comprising one or more members selected from the group consisting of a homeobox protein, a T-box transcription factor (TBX), a basic helix-loop-helix transcription factor (bHLH), a SOX, and a collagen.
104. The composition of 99, wherein the target gene is an endogenous target gene.
105. The composition of 99, wherein the target gene comprises a plurality of distinct target genes that comprises:
   (i) a first homeobox protein and a second homeobox protein that are different;
   (ii) two different members selected from the group consisting of a homeobox protein, a T-box transcription factor (TBX), and a basic helix-loop-helix transcription factor (bHLH); or
   (iii) a first member selected from the group consisting of the homeobox protein, the TBX, and the bHLH, and a second member comprising SOX or collagen.
106. The system or composition of any one of the preceding embodiments, wherein the plurality of chondrogenic cells comprises chondroprogenitor cells or chondrocytes.
107. The system or composition of any one of the preceding embodiments, wherein the plurality of chondrogenic cells is characterized to be CD146+/CD73+, CD146+/CD112+, or CD326+/CD309+.
108. The system or composition of any one of the preceding embodiments, wherein the plurality of stem cells comprise pluripotent stem cells (PSCs) or mesodermal cells.
109. The system or composition of any one of the preceding embodiments, wherein the plurality of distinct target genes are endogenous genes of the plurality of stem cells.
110. The system or composition of any one of the preceding embodiments, wherein the first heterologous gene regulating moiety or the second heterologous gene regulating moiety comprises (i) an endonuclease or (ii) a guide nucleic acid (gNA) molecule.
111. The system or composition of any one of the preceding embodiments, wherein the endonuclease and the gNA form a complex capable of binding to a respective target polynucleotide sequence.
112. The system or composition of any one of the preceding embodiments, wherein the endonuclease is a Cas protein.
113. The system or composition of any one of the preceding embodiments, wherein the conversion occurs in conditions substantially free of (i) serum and/or (ii) an exogenous cell differentiation regulatory factor.
114. The system or composition of any one of the preceding embodiments, wherein the exogenous cell differentiation regulatory factor comprises one or more members selected from the group consisting of TGFbeta1, TGFbeta2, TGFbeta3, BMP2, BMP4, BMP6, MP7, and IGF1.
115. The system or composition of any one of the preceding embodiments, wherein the exogenous cell differentiation regulatory factor is a chondrogenic factor comprising one or more members selected from the group consisting of dexamethasone, ascorbate, insulin, transferrin, and selenous acid.
116. The system or composition of any one of the preceding embodiments, wherein the first homeobox protein, the second homeobox protein, or the homeobox protein is a paired box (PRD)-class homeobox protein.
117. The system or composition of any one of the preceding embodiments, wherein the PRD-class homeobox protein is MIXL1 or UNCX.
118. The system or composition of any one of the preceding embodiments, wherein the first homeobox protein, the second homeobox protein, or the homeobox protein is not a member of Pax3 and Pax7.
119. The system or composition of any one of the preceding embodiments, wherein the TBX comprises one or more members selected from the group consisting of TBXT, TBR1, TBX1, TBX2, TBX3, TBX4, TBX5, TBX6, TBX10, TBX15, TBX18, TBX19, TBX20, TBX21, and TBX22.
120. The system or composition of any one of the preceding embodiments, wherein the TBX is TBXT or TBX6.
121. The system or composition of any one of the preceding embodiments, wherein the bHLH comprises one or more members selected from the group consisting of Group A bHLH, Group B bHLH, Group C bHLH, Group D bHLH, Group E bHLH, and Group F bHLH.
122. The system or composition of any one of the preceding embodiments, wherein the bHLH is the Group A bHLH.
123. The system or composition of any one of the preceding embodiments, wherein the bHLH is MSGN1 or TCF15.
124. The system or composition of any one of the preceding embodiments, wherein the SOX comprises one or more members selected from the group consisting of SOXA, SOXB1, SOXB2, SOXC, SOXD, SOXE, SOXF, SOXG, and SOXH.
125. The system or composition of any one of the preceding embodiments, wherein the SOX is SOXD.
126. The system or composition of any one of the preceding embodiments, wherein the SOXD is SOX6.
127. The system or composition of any one of the preceding embodiments, wherein the SOX is SOXE.
128. The system or composition of any one of the preceding embodiments, wherein the SOXE is SOX9.
129. The system or composition of any one of the preceding embodiments, wherein the collagen comprises one or more members selected from the group consisting of Type I collagen, Type II collagen, Type III collagen, Type IV collagen, and Type V collagen.
130. The system or composition of any one of the preceding embodiments, wherein the collagen is Type II collagen.
131. The system or composition of any one of the preceding embodiments, wherein the Type II collagen is COL2A1.
132. The system or composition of any one of the preceding embodiments, wherein the conversion occurs in less than about 10 days, less than about 7 days, or less than about 5 days.

Systems and methods of the present disclosure may be combined with or modified by other systems and methods for cell programming, such as, for example, those described in International Patent Application No. PCT/US2018/052211, International Patent Application No. PCT/US2018/052211, International Patent Application No. PCT/US2023/028169, International Patent Application No. PCT/US2023/028255, and International Patent Application No. PCT/US2023/028033, each of which is incorporated herein by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-11. (canceled)

12. A method for conversion of a plurality of stem cells into a plurality of chondrogenic cells, via modulation of expression levels of a plurality of distinct target genes comprising a first distinct target gene and a second distinct target gene, the method comprising:
a) contacting a first polynucleotide sequence in the plurality of stem cells by a first heterologous gene regulating moiety to modulate expression level of the first distinct target gene that is operatively coupled to the first polynucleotide sequence; and
b) contacting a second polynucleotide sequence in the plurality of stem cells by a second heterologous gene regulating moiety, to modulate expression level of the second distinct target gene that is operatively coupled to the second polynucleotide sequence, wherein a combination of the first and second distinct target genes is:
i) a first homeobox protein and a second homeobox protein that are different;
ii) two different members selected from the group consisting of a homeobox protein, a T-box transcription factor (TBX), and a basic helix-loop-helix transcription factor (bHLH); or
iii) a first member selected from the group consisting of the homeobox protein, the TBX, and the bHLH, and a second member comprising SOX or collagen.

13. The method of claim 12, wherein the combination is (i) the first homeobox protein and the second homeobox protein.

14. The method of claim 12, wherein the combination is (ii) the two different members selected from the group consisting of the homeobox protein, the TBX, and the bHLH.

15. The method of claim 12, wherein the combination is (iii) the first member selected from the group consisting of the homeobox protein, the TBX, and the bHLH, and the second member comprising the SOX or the collagen.

16. The method of claim 12, wherein (b) is performed subsequent to (a), thereby to effect modulation of the first distinct target gene and the second distinct target gene in a sequential manner.

17. The method of claim 12, wherein (i) the first polynucleotide sequence is upstream to or encodes the first distinct target gene or (ii) the second polynucleotide sequence is upstream to or encodes the second distinct target gene.

18. The method of claim 12, wherein (i) the expression level of the first distinct target gene is enhanced upon the contacting by the first heterologous gene regulating moiety or (ii) the expression level of the second distinct target gene is enhanced upon the contacting by the second heterologous gene regulating moiety.

19. The method of claim 12, wherein the method comprises contacting the plurality of stem cells with a heterologous genetic circuit comprising a plurality of gate units, wherein the heterologous genetic circuit is activatable to induce the plurality of gate units to modulate expression levels of the plurality of distinct target genes in a sequential manner to effect the conversion, and wherein the plurality of gate units comprises:
(i) a first gate unit that is preconfigured to effect the first heterologous gene regulating moiety to modulate the expression level of the first distinct target gene; and
(ii) a second gate unit that is preconfigured to effect the second heterologous gene regulating moiety to modulate the expression level of the second distinct target gene,
wherein, upon activation of the heterologous genetic circuit, the plurality of gate units operates to effect the conversion.

20. The method of claim 12, wherein the conversion occurs in less than about 14 days, less than about 10 days, less than about 7 days, or less than about 5 days.

21.-30. (canceled)

31. A method for treating a subject in need thereof, the method comprising:
administering a plurality of chondrogenic cells to the subject, wherein the plurality of chondrogenic cells is prepared by subjecting a plurality of stem cells to ex vivo differentiation,
wherein, within less than 7 days of the ex vivo differentiation, a conversion rate from the plurality of stem cells to the plurality of chondrogenic cells is characterized to be at least about 30%.

32. The method of claim 31, wherein the conversion rate is at least about 40%, at least about 50%, at least about 60%, at least about 70%, or at least about 80%.

33. The method of claim 31, wherein the conversion rate is observed within less than about 6 days following the contacting or within less than about 5 days following the contacting.

34. The method of claim 31, wherein the plurality of chondrogenic cells is subjected to ex vivo culture for less than about 2 weeks, less than about 10 days, less than about 8 days, less than about 7 days, less than about 6 days, or less than about 5 days.

35. The method of claim 31, wherein the ex vivo differentiation comprises modulating expression level of a target gene comprising one or more members selected from the group consisting of a homeobox protein, a T-box transcription factor (TBX), a basic helix-loop-helix transcription factor (bHLH), a SOX, and a collagen.

36. The method of claim 35, wherein the target gene is an endogenous target gene.

37. The method of claim 35, wherein the target gene comprises a plurality of distinct target genes that comprises:
(i) a first homeobox protein and a second homeobox protein that are different;
(ii) two different members selected from the group consisting of a homeobox protein, a T-box transcription factor (TBX), and a basic helix-loop-helix transcription factor (bHLH); or
(iii) a first member selected from the group consisting of the homeobox protein, the TBX, and the bHLH, and a second member comprising SOX or collagen.

38. The method of claim 12, wherein the plurality of chondrogenic cells comprises chondroprogenitor cells or chondrocytes.

39. The method of claim 12, wherein the plurality of chondrogenic cells is characterized to be CD146+/CD73+, CD146+/CD112+, or CD326+/CD309+.

40. The method of claim 12, wherein the plurality of stem cells comprise pluripotent stem cells (PSCs) or mesodermal cells.

41.-68. (canceled)

69. A system for conversion of a plurality of stem cells into a plurality of chondrogenic cells, via modulation of expression levels of a plurality of distinct target genes comprising a first distinct target gene and a second distinct target gene, the system comprising:
a. a first heterologous gene regulating moiety configured to bind a first polynucleotide sequence in the plurality of stem cells to modulate expression level of the first distinct target gene that is operatively coupled to the first polynucleotide sequence; and
b. a second heterologous gene regulating moiety configured to bind a second polynucleotide sequence in the plurality of stem cells to modulate expression level of the second distinct target gene that is operatively coupled to the second polynucleotide sequence,
wherein a combination of the first and second distinct target genes is:
(i) a first homeobox protein and a second homeobox protein that are different;
(ii) two different members selected from the group consisting of a homeobox protein, a T-box transcription factor (TBX), and a basic helix-loop-helix transcription factor (bHLH); or
(iii) first member selected from the group consisting of the homeobox protein, the TBX, and the bHLH, and a second member comprising SOX or collagen.

70.-71. (canceled)
